(19) United States
(12) Patent Application Publication
(10) Pub. No.: US 2018/0085387 A1
(43) Pub. Date: Mar. 29, 2018

(54) THERAPEUTIC COMPOSITIONS FOR TREATMENT OF HUMAN IMMUNODEFICIENCY VIRUS

(71) Applicant: Gilead Sciences, Inc., Foster City, CA (US)

(72) Inventors: Norbert W. Bischofberger, Hillsborough, CA (US); Andrew Cheng, San Francisco, CA (US); William A. Lee, Foster City, CA (US); Diana Sperger, Belmont, CA (US); Monica Tijerina, Foster City, CA (US)

(21) Appl. No.: 15/716,249

(22) Filed: Sep. 26, 2017

Related U.S. Application Data
(60) Provisional application No. 62/533,490, filed on Jul. 17, 2017.
(60) Provisional application No. 62/454,543, filed on Feb. 3, 2017.
(60) Provisional application No. 62/400,354, filed on Sep. 27, 2016.

(51) Int. Cl. A61K 31/7076; A61K 31/426; A61K 31/42; A61K 31/4995; A61K 31/4375; A61K 31/27; A61K 31/664; A61K 31/18

(52) U.S. Cl. CPC A61K 31/7076; A61K 31/426; A61K 31/42; A61K 31/18; A61K 31/4375; A61K 31/27; A61K 31/664; A61K 31/4995

(57) ABSTRACT

Pharmaceutical formulations suitable for treating viral infections such as HIV are provided, in particular solid oral dosage forms including the compounds of Formula I, Formula II, Formula III, Formula IV, or pharmaceutically acceptable salts or solvates thereof, and one or more excipients.

### CROSS-REFERENCE TO RELATED APPLICATIONS

This application claims the benefit of and priority to U.S. Provisional Patent Application Ser. No. 62/400,354, filed Sep. 27, 2016, 62/454,543, filed Feb. 3, 2017, and 62/533,490, filed Jul. 17, 2017 the disclosure of each of which are hereby incorporated herein by reference in their entirety.

### FIELD

Pharmaceutical formulations suitable for treating viral infections such as HIV are provided, in particular solid oral dosage forms including the compound of Formula I, bictegravir, cobicistat, and darunavir, or any pharmaceutically acceptable salt or solvate of the forgoing.

### BACKGROUND

Human immunodeficiency virus, type 1 (HIV-1) infection is a life-threatening and serious disease of major public health significance, with approximately 35 million people infected worldwide (Joint United Nations Programme on HIV/AIDS (UNAIDS). Global report: UNAIDS report on the global AIDS epidemic, 2013). Standard of care for the treatment of HIV-1 infection uses combination antiretroviral therapy (ART) to suppress viral replication to below detectable limits, increase CD4 cell counts, and halt disease progression.

Despite the successes of potent and well-tolerated ART, mutations of the HIV-1 virus continue to occur in clinical settings. For example, some treatment-experienced patients on ART experience drug resistance. As a result, achieving virologic suppression in this patient population is complex. In view of these challenges, there remains a significant medical need for safe and effective new therapies that address virologic resistance. Moreover, new therapies for patients experiencing virologic resistance must also: (i) exhibit tolerability, long-term safety, and adherence (Costagliola D. Demographics of HIV and aging Curr. Opin. HIV AIDS, 2014, 9(4), 294); as well as (ii) consider the aging patient population, non-HIV-related comorbidities, and regimen simplification.

### SUMMARY

Disclosed herein are compositions and oral dosage forms (e.g., tablets) comprising: (a) a compound of Formula I; (b) a compound of Formula II; (c) a compound of Formula III; and (d) a compound of Formula IV; or any pharmaceutically acceptable salt or solvate of the forgoing.

The compositions and oral dosage forms herein include a compound of Formula I, ethyl ((S)-((((2R,5R)-5-(6-amino-9H-purin-9-yl)-4-fluoro-2,5-dihydrofuran-2-yl)oxy)methyl)(phenoxy)phosphoryl)-l-alaninate, having the following structure:

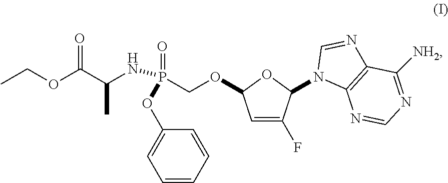 [SMILES: CCOC(=O)[C@H](C)N[P@](=O)(CO[C@@H]1C=C(F)[C@H](n2cnc3c(N)ncnc32)O1)Oc1ccccc1]

or a pharmaceutically acceptable salt thereof.

In some embodiments, the pharmaceutically acceptable salt is a compound of Formula Ia, ethyl ((S)-((((2R,5R)-5-(6-amino-9H-purin-9-yl)-4-fluoro-2,5-dihydrofuran-2-yl)oxy)methyl)(phenoxy)phosphoryl)-L-alaninate 2-hydroxypropane-1,2,3-tricarboxylate, having the following structure:

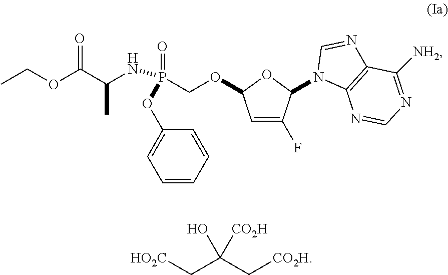 [SMILES: CCOC(=O)[C@H](C)N[P@](=O)(CO[C@@H]1C=C(F)[C@H](n2cnc3c(N)ncnc32)O1)Oc1ccccc1 ; O=C(O)CC(O)(CC(=O)O)C(=O)O]

In some embodiments, the compound of Formula I is a free base.

In some embodiments, the compositions and oral dosage forms herein include a compound of Formula II, (2R,5S,13aR)-8-hydroxy-7,9-dioxo-N-(2,4,6-trifluorobenzyl)-2,3,4,5,7,9,13,13 a-octahydro-2,5-methanopyrido [1′,2′:4,5]pyrazino[2,1-b][1,3]oxazepine-10-carboxamide (bictegravir), having the following structure:

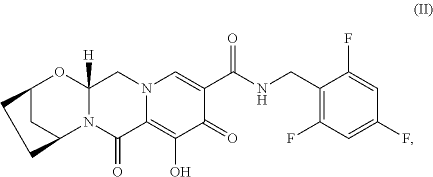 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof.

In some embodiments, the pharmaceutically acceptable salt is a compound of Formula IIa, sodium(2R,5S,13aR)-7,9-dioxo-10-((2,4,6-trifluorobenzyl)carbamoyl)-2,3,4,5,7,9,13,13 a-octahydro-2,5-methanopyrido[1′,2′:4,5]pyrazino[2,1-b][1,3]oxazepin-8-olate, having the following structure:

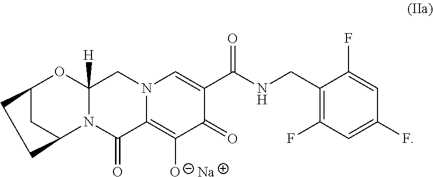 [SMILES: O=C(NCc1c(F)cc(F)cc1F)c1cn2c(c([O-])c1=O)C(=O)N1[C@H]3CC[C@H](C3)O[C@@H]1C2 ; [Na+]]

In some embodiments, the compositions and oral dosage forms herein include a compound of Formula III, 1,3-thiazol-5-ylmethyl[(2R,5R)-5-{[(2S)2-[(methyl{[2-(propan-2-yl)-1,3-thiazol-4-yl]methyl}carbamoyl)amino]-4-(morpholin-4 yl)butanoyl]amino}-1,6-diphenylhexan-2-yl]carbamate(cobicistat), having the following structure:

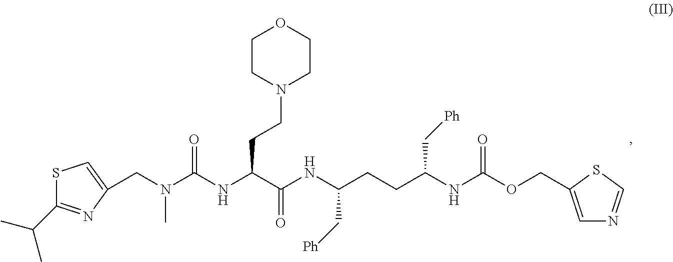 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt thereof.

In some embodiments, the compositions and oral dosage forms herein include a compound of Formula IV, [(1S,2R)-3-[[(4-aminophenyl)sulfonyl](2-methylpropyl)amino]-2-hydroxy-1-(phenylmethyl)propyl]-carbamic acid(3R,3aS,6aR)-hexahydrofuro[2,3-b]furan-3-yl ester(darunavir), having the following structure:

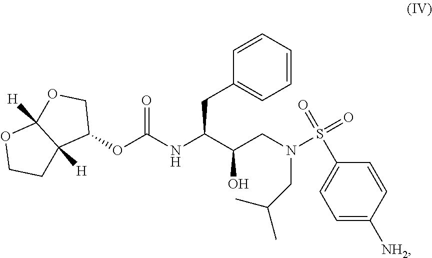 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

or a pharmaceutically acceptable salt or solvate thereof.

In some embodiments, the compound of Formula IV is a free base.

In some embodiments, the solvate is a compound of Formula IVa, [(1S,2R)-3-[[(4-aminophenyl)sulfonyl](2-methylpropyl)amino]-2-hydroxy-1-(phenylmethyl)propyl]-carbamic acid (3R,3aS,6aR)-hexahydrofuro[2,3-b]furan-3-yl ester monoethanolate, having the following structure:

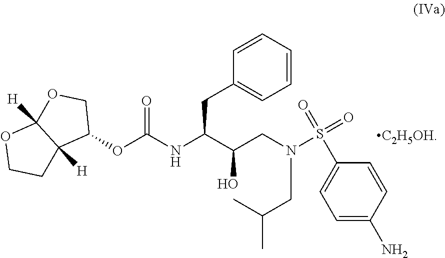 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

The oral dosage forms disclosed herein are suitable for use in medicine, and in particular in treating viral infections such as HIV. Accordingly, methods for treating patients are provided, which are also discussed in more detail below.

Methods of producing solid oral dosage forms such as tablets are also provided, as discussed in more detail below.

In some embodiments, a solid oral dosage form comprising: (a) the compound of Formula I or a pharmaceutically acceptable salt thereof, (b) the compound of Formula II or a pharmaceutically acceptable salt thereof, (c) the compound of Formula III or a pharmaceutically acceptable salt thereof, and (d) the compound of Formula IV or a pharmaceutically acceptable salt or solvate thereof, is provided. For instance, in some embodiments, the dosage form comprises 12-48 mg of the compound of Formula I or a pharmaceutically acceptable salt thereof, 20-80 mg of bictegravir or a pharmaceutically acceptable salt thereof, 60-240 mg of cobicistat or a pharmaceutically acceptable salt thereof, and 320-1280 mg of darunavir or a pharmaceutically acceptable salt or solvate thereof.

In some embodiments, the solid oral dosage form comprises 20-40 mg of the compound of Formula I or a pharmaceutically acceptable salt thereof, 40-60 mg of bictegravir or a pharmaceutically acceptable salt thereof, 140-160 mg cobicistat or a pharmaceutically acceptable salt thereof, and 790-810 mg darunavir or a pharmaceutically acceptable salt or solvate thereof. In some embodiments, the solid oral dosage form comprises 25-35 mg of the compound of Formula I or a pharmaceutically acceptable salt thereof, 45-55 mg of bictegravir or a pharmaceutically acceptable salt thereof, 145-155 mg cobicistat or a pharmaceutically acceptable salt thereof, and 795-805 mg darunavir or a pharmaceutically acceptable salt or solvate thereof.

In some embodiments, the solid oral dosage form comprises 30 mg of the compound of Formula I or a pharmaceutically acceptable salt thereof, 50 mg of bictegravir or a pharmaceutically acceptable salt thereof, 150 mg of cobicistat or a pharmaceutically acceptable salt thereof, and 800 mg of darunavir or a pharmaceutically acceptable salt or solvate thereof.

In one embodiment, a solid oral dosage form is provided, the solid dosage form including:
(a) about 0.5% to about 3% w/w of a compound of Formula I:

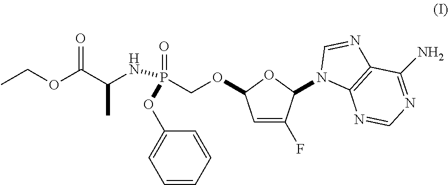 [SMILES: CCOC(=O)[C@H](C)N[P@](=O)(CO[C@@H]1C=C(F)[C@H](n2cnc3c(N)ncnc32)O1)Oc1ccccc1]

or a pharmaceutically acceptable salt thereof;
(b) about 2% to about 6% w/w of a compound of Formula II:

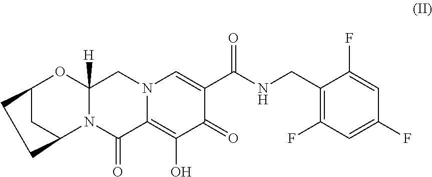 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof;
(c) about 10% to about 30% w/w a compound of Formula III:

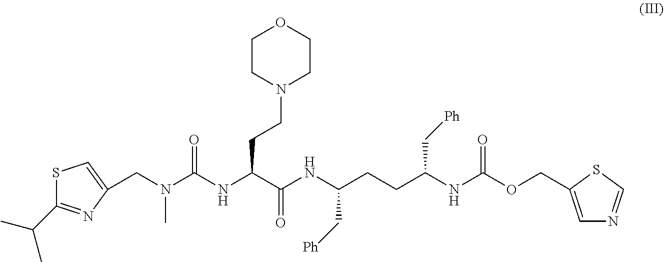 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt thereof; and
(d) about 40% to about 75% w/w of a compound of Formula IV:

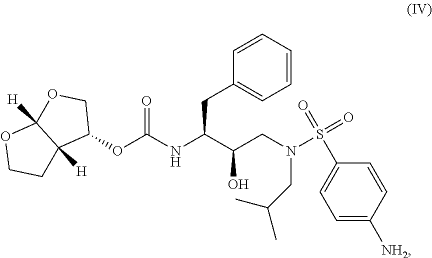 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

or a pharmaceutically acceptable salt or solvate thereof.

In one embodiment, a solid oral dosage form is provided, where the solid dosage form includes:
(a) about 0.5% to about 3% w/w of a compound of Formula I:

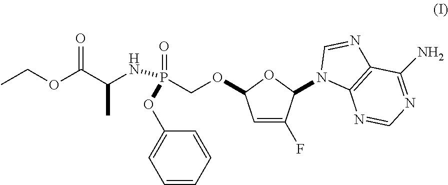 [SMILES: CCOC(=O)[C@H](C)N[P@](=O)(CO[C@@H]1C=C(F)[C@H](n2cnc3c(N)ncnc32)O1)Oc1ccccc1]

or a pharmaceutically acceptable salt thereof;
(b) about 2% to about 6% w/w of a compound of Formula II:

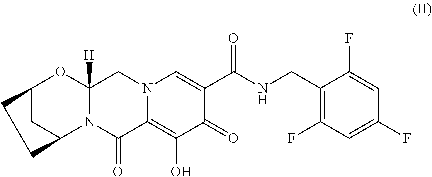 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof;
(c) about 5% to about 25% w/w a compound of Formula III:

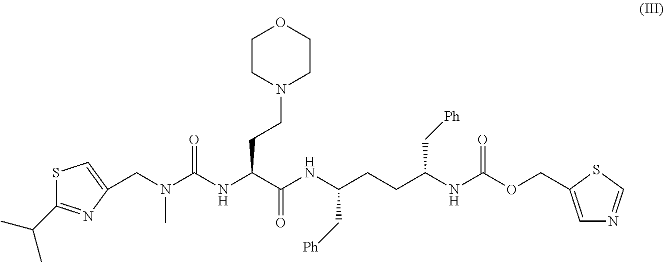 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt thereof; and
(d) about 40% to about 75% w/w of a compound of Formula IV:

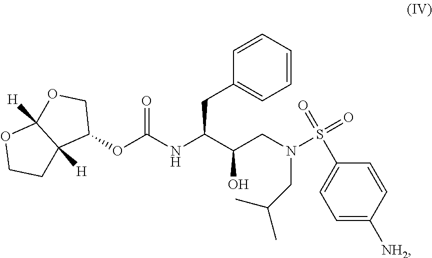 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

or a pharmaceutically acceptable salt or solvate thereof.

In one embodiment, a solid oral dosage form is provided, the solid dosage form including:
(a) about 0.5% to about 1% w/w of a compound of Formula I, or a pharmaceutically acceptable salt thereof;
(b) about 2.5% to about 4.5% w/w of a compound of Formula II, or a pharmaceutically acceptable salt thereof;
(c) about 15% to about 23% w/w a compound of Formula III, or a pharmaceutically acceptable salt thereof;
(d) about 50% to about 70% w/w of a compound of Formula IV, or a pharmaceutically acceptable salt or solvate thereof.

In one embodiment, the compound of Formula III, or a pharmaceutically acceptable salt thereof includes silicon dioxide particles.

In one embodiment, a solid oral dosage form is provided, the solid dosage form including:
(a) about 0.6% to about 0.8% w/w of a compound of Formula I, or a pharmaceutically acceptable salt thereof;
(b) about 3% to about 4% w/w of a compound of Formula II, or a pharmaceutically acceptable salt thereof;
(c) about 18% to about 22% w/w a compound of Formula III, or a pharmaceutically acceptable salt thereof;
(d) about 50% to about 65% w/w of a compound of Formula IV, or a pharmaceutically acceptable salt or solvate thereof.

In one embodiment, the compound of Formula III, or a pharmaceutically acceptable salt thereof includes silicon dioxide particles.

In one embodiment, a solid oral dosage form is provided where the solid dosage form includes:
(a) about 1.0% to about 3.1% w/w of a compound of Formula I, or a pharmaceutically acceptable salt thereof;
(b) about 1.0% to about 4.0% w/w of a compound of Formula II, or a pharmaceutically acceptable salt thereof;
(c) about 4% to about 18% w/w a compound of Formula III, or a pharmaceutically acceptable salt thereof;
(d) about 50% to about 70% w/w of a compound of Formula IV, or a pharmaceutically acceptable salt or solvate thereof.

In one embodiment, the compound of Formula III, or a pharmaceutically acceptable salt thereof may be adsorbed onto a solid carrier. In certain embodiments, the solid carrier is silicon dioxide particles (i.e., silica). In certain embodiments, the compound of Formula III is in a crystalline form as characterized by its X-ray diffraction pattern.

In one embodiment, a solid oral dosage form is provided where the solid dosage form includes:
(a) about 1.5% to about 2.5% w/w of a compound of Formula I, or a pharmaceutically acceptable salt thereof;
(b) about 2.0% to about 3.0% w/w of a compound of Formula II, or a pharmaceutically acceptable salt thereof;
(c) about 7% to about 12% w/w a compound of Formula III, or a pharmaceutically acceptable salt thereof;
(d) about 50% to about 65% w/w of a compound of Formula IV, or a pharmaceutically acceptable salt or solvate thereof.

In one embodiment, the compound of Formula III, or a pharmaceutically acceptable salt thereof includes silicon dioxide particles (i.e., silica). In certain embodiments, the compound of Formula III is in a crystalline form as characterized by its X-ray diffraction pattern.

In one embodiment, a solid oral dosage form is provided where the solid dosage form includes:
(a) about 1.0% to about 3.1% w/w of the compound of Formula I, or a pharmaceutically acceptable salt thereof;
(b) about 1.0% to about 4.0% w/w of the compound of Formula II, or a pharmaceutically acceptable salt thereof;
(c) about 15% to about 25% w/w the compound of Formula III, or a pharmaceutically acceptable salt thereof, and wherein the compound of Formula III is adsorbed onto a solid carrier;
(d) about 50% to about 70% w/w of the compound of Formula IV, or a pharmaceutically acceptable salt or solvate thereof.

In one embodiment, a solid oral dosage form is provided, the solid dosage form including:
(a) about 10 mg to about 30 mg of a compound of Formula I, or a pharmaceutically acceptable salt thereof;
(b) about 25 mg to about 75 mg of a compound of Formula II, or a pharmaceutically acceptable salt thereof;
(c) about 150 mg to about 350 mg of a compound of Formula III, or a pharmaceutically acceptable salt thereof;
(d) about 600 mg to about 1000 mg of a compound of Formula IV, or a pharmaceutically acceptable salt or solvate thereof;
(e) about 100 mg to about 175 mg of microcrystalline cellulose;
(f) about 65 mg to about 105 mg of crospovidone; and
(g) about 10 mg to about 30 mg of magnesium stearate.

The solid oral dosage forms disclosed herein include about 125-175 mg, or about 140-160 mg, or about 139 mg silica particles (e.g., silicon dioxide).

In one embodiment, a solid oral dosage form is provided, the solid dosage form including:
a) about 10 mg to about 15 mg of a compound of Formula I, or a pharmaceutically acceptable salt thereof;
b) about 40 mg to about 60 mg of a compound of Formula II, or a pharmaceutically acceptable salt thereof;
c) about 230 mg to about 320 mg of a combination comprising the compound of Formula III, or a pharmaceutically acceptable salt thereof, and silicon dioxide particles;
d) about 700 mg to about 900 mg of a compound of Formula IV, or a pharmaceutically acceptable salt or solvate thereof;
e) about 110 mg to about 165 mg of microcrystalline cellulose;
f) about 75 mg to about 100 mg of crospovidone; and
g) about 15 mg to about 25 mg of magnesium stearate.

In one embodiment, a solid oral dosage form is provided, the solid dosage form including:
a) about 10 mg to about 12 mg of a compound of Formula I, or a pharmaceutically acceptable salt thereof;
b) about 45 mg to about 55 mg of a compound of Formula II, or a pharmaceutically acceptable salt thereof;
c) about 260 mg to about 310 mg of a combination comprising a compound of Formula III, or a pharmaceutically acceptable salt thereof and silicon dioxide particles;
d) about 800 mg to about 900 mg of a compound of Formula IV, or a pharmaceutically acceptable salt or solvate thereof;
e) about 115 mg to about 155 mg of microcrystalline cellulose;
f) about 83 mg to about 93 mg of crospovidone; and
g) about 18 mg to about 24 mg of magnesium stearate.

In one embodiment, a solid oral dosage form is provided where the solid dosage form includes:
(a) about 10 mg to about 45 mg of a compound of Formula I, or a pharmaceutically acceptable salt thereof;
(b) about 15 mg to about 50 mg of a compound of Formula II, or a pharmaceutically acceptable salt thereof;
(c) about 50 mg to about 400 mg of a compound of Formula III, or a pharmaceutically acceptable salt thereof;
(d) about 500 mg to about 1200 mg of a compound of Formula IV, or a pharmaceutically acceptable salt or solvate thereof;
(e) about 100 mg to about 175 mg of microcrystalline cellulose;
(f) about 50 mg to about 140 mg of crospovidone; and
(g) about 5 mg to about 25 mg of magnesium stearate.

In certain embodiments of the solid oral dosage forms, the amount of silicon dioxide particles is about 125-175 mg, or about 140-160 mg, or about 139 mg.

In one embodiment, a solid oral dosage form is provided where the solid dosage form includes:
(a) about 20 mg to about 40 mg of a compound of Formula I, or a pharmaceutically acceptable salt thereof;
(b) about 20 mg to about 45 mg of a compound of Formula II, or a pharmaceutically acceptable salt thereof;
(c) about 125 mg to about 250 mg of a compound of Formula III, or a pharmaceutically acceptable salt thereof, and silicon dioxide particles;
(d) about 550 mg to about 1100 mg of a compound of Formula IV, or a pharmaceutically acceptable salt or solvate thereof;
(e) about 90 mg to about 165 mg of microcrystalline cellulose;
(f) about 60 mg to about 110 mg of crospovidone; and
(g) about 8 mg to about 20 mg of magnesium stearate.

In one embodiment, a solid oral dosage form is provided where the solid dosage form includes:
(a) about 28 mg to about 32 mg of a compound of Formula I, or a pharmaceutically acceptable salt thereof;
(b) about 25 mg to about 37 mg of a compound of Formula II, or a pharmaceutically acceptable salt thereof;
(c) about 135 mg to about 180 mg of a compound of Formula III, or a pharmaceutically acceptable salt thereof;
(d) about 700 mg to about 1000 mg of a compound of Formula IV, or a pharmaceutically acceptable salt or solvate thereof;
(e) about 100 mg to about 160 mg of microcrystalline cellulose;
(f) about 70 mg to about 100 mg of crospovidone; and
(g) about 12 mg to about 16 mg of magnesium stearate.

In one embodiment, a solid oral dosage form is provided where the solid dosage form includes:
(a) about 28 mg to about 32 mg of a compound of Formula I, or a pharmaceutically acceptable salt thereof;
(b) about 25 mg to about 37 mg of a compound of Formula II, or a pharmaceutically acceptable salt thereof;
(c) about 135 mg to about 180 mg of a compound of Formula III, or a pharmaceutically acceptable salt thereof;
(d) about 700 mg to about 1000 mg of a compound of Formula IV, or a pharmaceutically acceptable salt or solvate thereof;
(e) about 70 mg to about 100 mg of microcrystalline cellulose;
(f) about 70 mg to about 100 mg of crospovidone; and
(g) about 12 mg to about 16 mg of magnesium stearate.

In one embodiment, a solid oral dosage form is provided where the solid dosage form includes:
(a) about 28 mg to about 32 mg of the compound of Formula I, or a pharmaceutically acceptable salt thereof;
(b) about 25 mg to about 37 mg of the compound of Formula II, or a pharmaceutically acceptable salt thereof;
(c) about 230 mg to about 350 mg of the compound of Formula III, or a pharmaceutically acceptable salt thereof adsorbed onto a solid carrier;
(d) about 700 mg to about 1000 mg of the compound of Formula IV, or a pharmaceutically acceptable salt or solvate thereof;
(e) about 100 mg to about 150 mg of microcrystalline cellulose;
(f) about 60 mg to about 110 mg of crospovidone; and
(g) about 5 mg to about 20 mg of magnesium stearate.

In some embodiments of a solid oral dosage form described includes:
a) about 2.5% to about 6.0% w/w of a compound of Formula I:

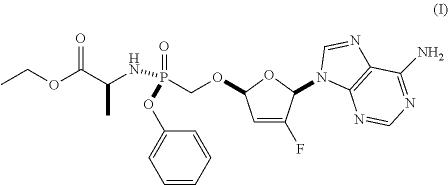 [SMILES: CCOC(=O)[C@H](C)N[P@](=O)(CO[C@@H]1C=C(F)[C@H](n2cnc3c(N)ncnc32)O1)Oc1ccccc1]

or a pharmaceutically acceptable salt thereof;
b) about 2% to about 5% w/w of a compound of Formula II:

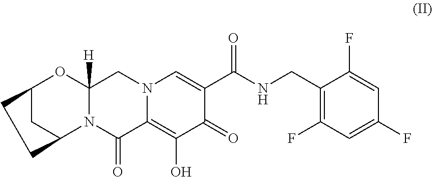 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof;
c) about 5% to about 25% w/w a compound of Formula III:

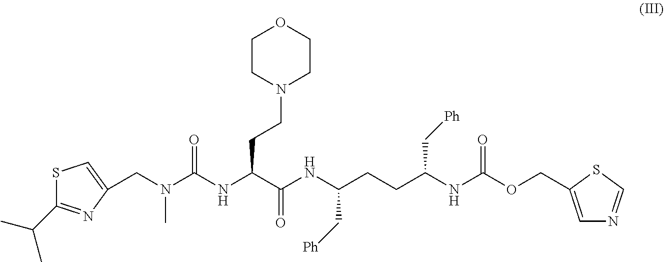 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt thereof; and
d) about 40% to about 75% w/w of a compound of Formula IV:

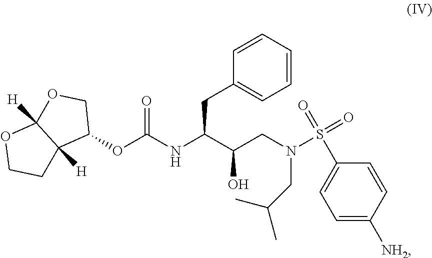 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

or a pharmaceutically acceptable salt or solvate thereof.

In one embodiment, a solid oral dosage form is provided where the solid dosage form includes:
(a) about 2.5% to about 5.8% w/w of a compound of Formula I, or a pharmaceutically acceptable salt thereof;
(b) about 1.8% to about 4.2% w/w of a compound of Formula II, or a pharmaceutically acceptable salt thereof;
(c) about 4% to about 18% w/w a compound of Formula III, or a pharmaceutically acceptable salt thereof;
(d) about 50% to about 70% w/w of a compound of Formula IV, or a pharmaceutically acceptable salt or solvate thereof.

In one embodiment, a solid oral dosage form is provided where the solid dosage form includes:
(a) about 3.5% to about 5.0% w/w of a compound of Formula I, or a pharmaceutically acceptable salt thereof;
(b) about 2.0% to about 3.0% w/w of a compound of Formula II, or a pharmaceutically acceptable salt thereof;
(c) about 7.0% to about 13.0% w/w a compound of Formula III, or a pharmaceutically acceptable salt thereof;
(d) about 55% to about 62% w/w of a compound of Formula IV, or a pharmaceutically acceptable salt or solvate thereof.

In one embodiment, a solid oral dosage form is provided where the solid dosage form includes:
(a) about 42 mg to about 78 mg of a compound of Formula I, or a pharmaceutically acceptable salt thereof;
(b) about 22 mg to about 40 mg of a compound of Formula II, or a pharmaceutically acceptable salt thereof;
(c) about 130 mg to about 200 mg of a compound of Formula III, or a pharmaceutically acceptable salt thereof;
(d) about 600 mg to about 1100 mg of a compound of Formula IV, or a pharmaceutically acceptable salt or solvate thereof;
(e) about 73 mg to about 135 mg of microcrystalline cellulose;
(f) about 60 mg to about 110 mg of crospovidone; and
(g) about 5 mg to about 20 mg of magnesium stearate.

In one embodiment, a solid oral dosage form is provided, the solid dosage form including:
(a) about 54 mg to about 66 mg of a compound of Formula I, or a pharmaceutically acceptable salt thereof;
(b) about 28 mg to about 35 mg of a compound of Formula II, or a pharmaceutically acceptable salt thereof;
(c) about 140 mg to about 170 mg of a compound of Formula III, or a pharmaceutically acceptable salt thereof;
(d) about 780 mg to about 950 mg of a compound of Formula IV, or a pharmaceutically acceptable salt or solvate thereof;
(e) about 90 mg to about 115 mg of microcrystalline cellulose;
(f) about 75 mg to about 95 mg of crospovidone; and
(g) about 5 mg to about 17 mg of magnesium stearate.

In one embodiment, a solid oral dosage form is provided where the solid dosage form includes:
(a) about 59 mg to about 62 mg of a compound of Formula I, or a pharmaceutically acceptable salt thereof;
(b) about 30 mg to about 35 mg of a compound of Formula II, or a pharmaceutically acceptable salt thereof;
(c) about 145 mg to about 160 mg of a compound of Formula III, or a pharmaceutically acceptable salt thereof;
(d) about 800 mg to about 950 mg of a compound of Formula IV, or a pharmaceutically acceptable salt or solvate thereof;
(e) about 99 mg to about 105 mg of microcrystalline cellulose;
(f) about 85 mg to about 90 mg of crospovidone; and
(g) about 6 mg to about 15 mg of magnesium stearate.

In one embodiment, a solid oral dosage form is provided where the solid dosage form includes:
(a) about 59 mg to about 62 mg of a compound of Formula I, or a pharmaceutically acceptable salt thereof;
(b) about 30 mg to about 35 mg of a compound of Formula II, or a pharmaceutically acceptable salt thereof;
(a) about 142 mg to about 160 mg of a compound of Formula III, or a pharmaceutically acceptable salt thereof;
(b) about 800 mg to about 950 mg of a compound of Formula IV, or a pharmaceutically acceptable salt or solvate thereof;
(c) about 99 mg to about 105 mg of microcrystalline cellulose;
(d) about 85 mg to about 90 mg of crospovidone; and
(e) about 6 mg to about 15 mg of magnesium stearate.

In one embodiment, a solid oral dosage form is provided where the solid dosage form includes:
(a) about 54 mg to about 66 mg of the compound of Formula I, or a pharmaceutically acceptable salt thereof;
(b) about 28 mg to about 35 mg of the compound of Formula II, or a pharmaceutically acceptable salt thereof;
(c) about 260 mg to about 310 mg of the compound of Formula III, or a pharmaceutically acceptable salt thereof adsorbed onto a solid carrier;
(d) about 780 mg to about 950 mg of the compound of Formula IV, or a pharmaceutically acceptable salt or solvate thereof;
(e) about 90 mg to about 115 mg of microcrystalline cellulose;
(f) about 75 mg to about 95 mg of crospovidone; and
(g) about 10 mg to about 20 mg of magnesium stearate.

In any of the embodiments of the solid oral dosage form described herein, the compound of Formula III, or a pharmaceutically acceptable salt thereof is adsorbed onto a solid carrier. In certain embodiments of the solid oral dosage form, the solid carrier is made up of silica particles. In certain embodiment of the solid oral dosage form where the compound of Formula III is adsorbed onto a solid carrier, the solid carrier is made up of silica articles. In certain embodiments of the solid oral dosage form, Formula III is adsorbed onto a solid carrier where the solid carrier is made up of silicon dioxide particles. In certain embodiments of the solid oral dosage form, the solid carrier is approximately 100-200 mgs. In certain embodiments of the solid oral dosage form, the solid carrier is approximately ten percent of the weight of the solid oral dosage form. In certain embodiments of the solid oral dosage form, the solid carrier is less than approximately ten percent of the weight of the solid oral dosage form.

In any of the embodiments of the solid oral dosage form described above, the compound of Formula III, or a pharmaceutically acceptable salt thereof may be in a crystalline form as characterized by its distinct X-ray diffraction pattern.

The solid oral dosage forms described herein may be in the form of a tablet.

In some embodiments, the solid oral dosage forms described herein may further include a film coating.

In some embodiments, the solid oral dosage forms described herein will have a total weight of approximately 1.5 grams.

In some embodiments of the solid oral dosage forms, the total weight of the solid oral dosage forms is approximately between 1400 mg and 1600 mg.

In some embodiments, the solid oral dosage form may contain at least about 75% w/w ratio of compound having Formula I or a pharmaceutically acceptable salt thereof, the compound of Formula II or a pharmaceutically acceptable salt thereof, the compound of Formula III or a pharmaceutically acceptable salt thereof, and the compound of Formula IV or a pharmaceutically acceptable salt or solvate thereof to the solid oral dosage form including excipients.

In some embodiments of the solid oral dosage form, the dosage form includes less than approximately 600 mg of excipients. In some other embodiments of the solid oral dosage form, the dosage form includes less than approximately 300 mgs of excipients.

The inventors have found that the use of a fixed dose combination may assist in achieving appropriate pharmacokinetic parameters and/or adequate tablet stability. In addition, the use of a multilayer tablet as a particular type of fixed dose combination may also provide pharmacokinetic and/or stability benefits. Accordingly, in another aspect a fixed dose combination tablet comprising (a) the compound of Formula I, or a pharmaceutically acceptable salt thereof, (b) the compound of Formula II, or a pharmaceutically acceptable salt thereof, (c) cobicistat, or a pharmaceutically acceptable salt thereof, and (d) darunavir, or a pharmaceutically acceptable salt or solvate thereof is provided. Additionally, a multilayer tablet comprising (a) the compound of Formula I, or a pharmaceutically acceptable salt thereof, (b) the compound of Formula II, or a pharmaceutically acceptable salt thereof, (c) cobicistat, or a pharmaceutically acceptable salt thereof, and (d) darunavir, or a pharmaceutically acceptable salt or solvate thereof is provided.

In some embodiments, a kit comprising: (i) a tablet comprising a compound of Formula I (or a pharmaceutically acceptable salt thereof), a compound of Formula II (or a pharmaceutically acceptable salt thereof), cobicistat (or a pharmaceutically acceptable salt thereof), and darunavir (or a pharmaceutically acceptable salt or solvate thereof); and (ii) a desiccant (e.g. silica gel) is provided.

In addition, methods for treating patients are provided herein, which are also discussed in more detail below.

Methods of treatment incorporating the solid oral dosage forms disclosed herein are also provided, e.g., methods of therapeutic treatment of an HIV infection. In certain embodiments, the subject is a treatment-experienced subject. In certain embodiments, the treatment-experienced subject has a resistance mutation selected from a thymidine analogue mutation (TAM), M184V, K65R, and L74V.

### BRIEF DESCRIPTION OF DRAWINGS

FIG. 1 is a flow diagram illustrating the preparation of a tablet formulation containing the compounds of Formula I, Formula II, Formula III, and Formula IV.

FIG. 2 shows a comparison of mean concentration of Formula I over time dosed as a tablet formulation against co-administration of single agent Formula I, single agent Formula II, and co-formulated darunavir and cobicistat.

FIG. 3 shows a comparison of mean concentration of Formula II over time dosed as a tablet formulation against co-administration of single agent Formula I, single agent Formula II, and co-formulated darunavir and cobicistat.

FIG. 4 shows a comparison of mean concentration of Formula III over time dosed as a tablet formulation against co-administration of single agent Formula I, single agent Formula II, and co-formulated darunavir and cobicistat.

FIG. 5 shows a comparison of mean concentration of Formula IV over time dosed as a tablet formulation against co-administration of single agent Formula I, single agent Formula II, and co-formulated darunavir and cobicistat.

FIG. 6a shows percent release over time of Formula I in a tablet formulation having Formulation F4 (Formula I/Formula II/Formula III/Formula IV (30/30/150/800, w/w)).

FIG. 6b shows percent release over time of Formula I in a tablet formulation F6 (Formula I/Formula II/Formula III/Formula IV (60/30/150/800, w/w)).

FIG. 7a shows percent release over time of Formula II in a tablet formulation F4 (Formula I/Formula II/Formula III/Formula IV (30/30/150/800, w/w)).

FIG. 7b shows percent release over time of Formula II in a tablet formulation F6 (Formula I/Formula II/Formula III/Formula IV (60/30/150/800, w/w)).

FIG. 8a shows percent release over time of Formula III of a tablet formulation F4 (Formula I/Formula II/Formula III/Formula IV (30/30/150/800, w/w)).

FIG. 8b shows percent release over time of Formula III of a tablet formulation F6 (Formula I/Formula II/Formula III/Formula IV (60/30/150/800, w/w)).

FIG. 9a shows percent release over time of Formula IV of a tablet formulation F4 (Formula I/Formula II/Formula III/Formula IV (30/30/150/800, w/w)).

FIG. 9b shows percent release over time of Formula IV of a tablet formulation F6 (Formula I/Formula II/Formula III/Formula IV (60/30/150/800, w/w)).

FIG. 10 shows Formula I degradation products as a function of pH.

FIG. 11 shows a series of X-ray powder diffraction spectra for Formula I over a 15 week period at 40° C. at 75% RH in a closed and in an open environment.

### DETAILED DESCRIPTION

The oral dosage forms disclosed herein comprise four active pharmaceutical ingredients: the compound of Formula I (or a pharmaceutically acceptable salt thereof), the compound of Formula II (or a pharmaceutically acceptable salt thereof), the compound of Formula III (or a pharmaceutically acceptable salt thereof), and the compound of Formula IV (or a pharmaceutically acceptable salt or solvate thereof).

Ethyl ((S)-((((2R,5R)-5-(6-amino-9H-purin-9-yl)-4-fluoro-2,5-dihydrofuran-2-yl)oxy)methyl)(phenoxy)phosphoryl)-L-alaninate

Ethyl ((S)-((((2R,5R)-5-(6-amino-9H-purin-9-yl)-4-fluoro-2,5-dihydrofuran-2-yl)oxy)methyl)(phenoxy)phosphoryl)-L-alaninate (Formula I), is a prodrug of an HIV reverse-transcriptase (RT) inhibitor. This compound has a favorable in vitro resistance profile with activity against Nucleoside RT Inhibitor (NRTI)-Resistance Mutations, such as M184V, K65R, L74V, and one or more (e.g., 1, 2, 3, or 4) TAMs (thymidine analogue mutations). It has the following formula (see, e.g., U.S. Pat. No. 7,871,991):

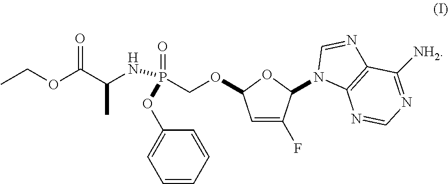 [SMILES: CCOC(=O)[C@H](C)N[P@](=O)(CO[C@@H]1C=C(F)[C@H](n2cnc3c(N)ncnc32)O1)Oc1ccccc1]

The compound of Formula I is a base and is susceptible to hydrolysis.

In some embodiments, solid oral dosage forms containing 6-48 mg of the compound of Formula I, or a pharmaceutically acceptable salt thereof, are provided. In some embodiments, solid oral dosage forms containing 12-48 mg of the compound of Formula I, or a pharmaceutically acceptable salt thereof, are provided. In some embodiments, solid oral dosage forms containing 8-40, or 15-45 mg, of the compound of Formula I, or a pharmaceutically acceptable salt thereof, are provided. In some embodiments, solid oral dosage forms containing 12-40 mg of the compound of Formula I, or a pharmaceutically acceptable salt thereof, are provided. In some embodiments, solid oral dosage forms containing 12-30 mg of the compound of Formula I, or a pharmaceutically acceptable salt thereof, are provided. In some embodiments, solid oral dosage forms containing 10-30 mg of the compound of Formula I, or a pharmaceutically acceptable salt thereof, are provided. In certain embodiments, the solid oral dosage form contains 30 mg of the compound of Formula I, or a pharmaceutically acceptable salt thereof.

In some embodiments, solid oral dosages containing 30-80 mg of the compound of Formula I, or a pharmaceutically acceptable salt thereof, are provided. In some embodiments, solid oral dosage forms containing 40-70 mg of the compound of Formula I, or a pharmaceutically acceptable salt thereof, are provided. In some embodiments, solid oral dosage forms containing 45-65 mg of the compound of Formula I, or a pharmaceutically acceptable salt thereof, are provided. In certain embodiments, the solid oral dosage form contains 60 mg of the compound of Formula I, or a pharmaceutically acceptable salt thereof.

Solid oral dosage forms disclosed herein include the compound of Formula I, or a pharmaceutically acceptable salt thereof. The compound of Formula I can be present within an oral dosage form in solvated or unsolvated form, and references to “Formula I” include both of these forms.

In some embodiments, the compound of Formula I is a free base. As it pertains to this application, when Formula I is disclosed, the free base form is intended unless otherwise noted.

In certain embodiments, the compound of Formula I is in the form of the compound of Formula Ia, having the formula below:

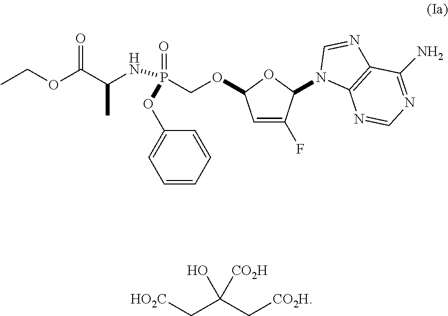 [SMILES: CCOC(=O)[C@H](C)N[P@](=O)(CO[C@@H]1C=C(F)[C@H](n2cnc3c(N)ncnc32)O1)Oc1ccccc1 ; O=C(O)CC(O)(CC(=O)O)C(=O)O]

One name for the compound of Formula (Ia) is ethyl ((S)-((((2R,5R)-5-(6-amino-9H-purin-9-yl)-4-fluoro-2,5-dihydrofuran-2-yl)oxy)methyl)(phenoxy)phosphoryl)-L-alaninate 2-hydroxypropane-1,2,3-tricarboxylate. Another name for the compound of Formula (Ia) is the citrate salt of ethyl ((S)-((((2R,5R)-5-(6-amino-9H-purin-9-yl)-4-fluoro-2,5-dihydrofuran-2-yl)oxy)methyl)(phenoxy)phosphoryl)-L-alaninate.

In some embodiments, the compound of Formula I is the vanillate (i.e., Formula Ib), having the following structure:

### Bictegravir

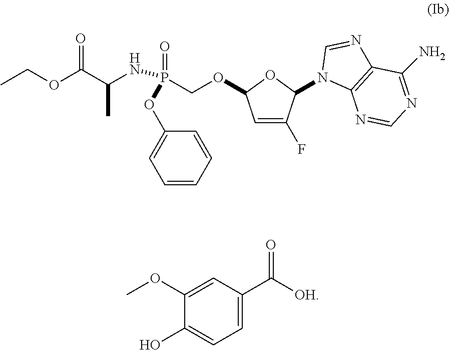 [SMILES: CCOC(=O)[C@H](C)N[P@](=O)(CO[C@@H]1C=C(F)[C@H](n2cnc3c(N)ncnc32)O1)Oc1ccccc1 ; COc1cc(C(=O)O)ccc1O]

(2R,5S,13aR)-8-Hydroxy-7,9-dioxo-N-(2,4,6-trifluorobenzyl)-2,3,4,5,7,9,13,13 a-octahydro-2,5-methanopyrido[1′,2′:4,5]pyrazino[2,1-b][1,3]oxazepine-10-carboxamide (Formula II), is a potent HIV integrase inhibitor with in vitro activity against wild type HIV-1. It has the following formula (see WO2014/100323):

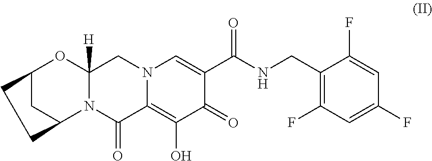 [SMILES: O=C(NCc1c(F)cc(F)cc1F)c1cn2c(c(O)c1=O)C(=O)N1[C@H]3CC[C@H](C3)O[C@@H]1C2]

Its IUPAC name is (2R,5S,13aR)-8-hydroxy-7,9-dioxo-N-(2,4,6-trifluorobenzyl)-2,3,4,5,7,9,13,13 a-octahydro-2,5-methanopyrido[1′,2′:4,5]pyrazino[2,1-b][1,3]oxazepine-10-carboxamide. Its CAS name is 2,5-Methanopyrido[1′,2′:4,5]pyrazino[2,1-b][1,3]oxazepine-10-carboxamide, 2,3,4,5,7,9,13,13 a-octahydro-8-hydroxy-7,9-dioxo-N-[2,4,6-trifluorophenyl)methyl]-, (2R,5S,13aR). The compound of Formula II is also referred to as bictegravir.

Solid oral dosage forms disclosed herein include the compound of Formula II, usually in the form of a pharmaceutically acceptable salt. The compound of Formula II can be present within an oral dosage form in solvated or unsolvated form, and references to “Formula II” include both of these forms. In certain embodiments, the compound of Formula II is in the form of the compound of Formula IIa, having the formula below:

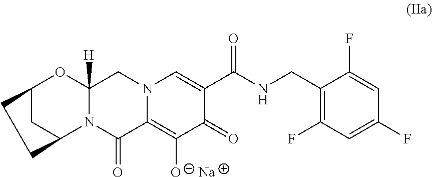 [SMILES: O=C(NCc1c(F)cc(F)cc1F)c1cn2c(c([O-])c1=O)C(=O)N1[C@H]3CC[C@H](C3)O[C@@H]1C2 ; [Na+]]

One name for the compound of Formula (IIa) is sodium (2R,5S,13aR)-7,9-dioxo-10-((2,4,6-trifluorobenzyl)carbamoyl)-2,3,4,5,7,9,13,13 a-octahydro-2,5-methanopyrido[1′,2′:4,5]pyrazino[2,1-b][1,3]oxazepin-8-olate.

In some embodiments, solid oral dosage forms containing 20-80 mg of the compound of Formula II, or a pharmaceutically acceptable salt thereof, are provided. In some embodiments, solid oral dosage forms containing 20-60 mg of the compound of Formula II, or a pharmaceutically acceptable salt thereof, are provided. In some embodiments, solid oral dosage forms containing 20-50 mg of the compound of Formula II, or a pharmaceutically acceptable salt thereof, are provided.

In some embodiments, solid oral dosage forms containing 25-75 mg, or 15-75 mg, of the compound of Formula II, or a pharmaceutically acceptable salt thereof, are provided. In some embodiments, solid oral dosage forms containing 50-75 mg, or 20-60 mg, of the compound of Formula II, or a pharmaceutically acceptable salt thereof, are provided. In some embodiments, solid oral dosage forms containing 25-45 mg of the compound of Formula II, or a pharmaceutically acceptable salt thereof, are provided.

### Cobicistat

Cobicistat is described in WO 2008/010921, incorporated herein by reference in its entirety, and has been shown to be a mechanism-based inhibitor of CYP3A enzymes, CYP3A4 and CYP3A5, with greater specificity than ritonavir. Xu et al., ACS Med. Chem. Lett. (2010), 1, pp. 209-13. The structure of cobicistat is shown below (Formula III):

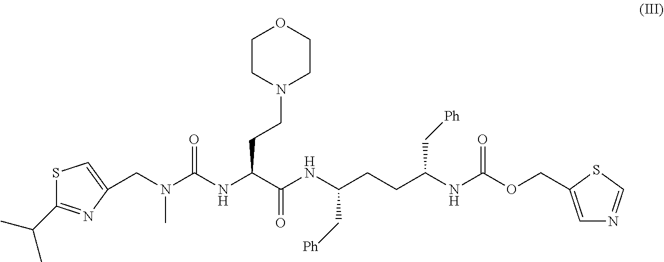 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

Cobicistat refers to 1,3-thiazol-5-ylmethyl (2R,5R)-(5-{[(2S)-2-[(methyl{[2-(propan-2-yl)-1,3-thiazol-4-yl]methyl}carbamoyl)amino]]-4-(morpholin-4-yl)butanamido}-1,6-diphenylhexan-2-yl)carbamate). It is currently authorized as part of products such as TYBOST® (cobicistat 150 mg), STRIBILD® (emtricitabine 200 mg, cobicistat 150 mg, tenofovir disoproxil fumarate 300 mg, elvitegravir 150 mg), GENVOYA® (emtricitabine 200 mg, cobicistat 150 mg, tenofovir alafenamide 10 mg, elvitegravir 150 mg), and PREZCOBIX ((darunavir 800 mg and cobicistat 150 mg).

Solid oral dosage forms disclosed herein include cobicistat. Cobicistat can be present within an oral dosage form in solvated or unsolvated form, and references to “cobicistat” include both of these forms. The compound of Formula III is also referred to as cobicistat.

In some embodiments, cobicistat is in a crystalline form. Crystalline forms of cobicistat are disclosed in U.S. patent application Ser. No. 15/414,438 entitled Crystalline Form (filed Jan. 24, 2017). In certain embodiments, cobicistat has a crystalline form characterized by having an X-ray powder diffraction (XRPD) pattern with peaks at 17.2±0.2 and 19.6±0.2 (Cu Kα radiation, expressed in degrees 2 θ). In other embodiments, cobicistat has a crystalline form characterized by having an X-ray powder diffraction (XRPD) pattern comprising peaks at 13.5±0.2, 17.2±0.2, 19.6±0.2 and 20.8±0.2 (Cu Kα radiation, expressed in degrees 2 θ). In other embodiments, cobicistat has a crystalline form characterized by having an X-ray powder diffraction (XRPD) pattern comprising peaks at 7.0±0.2, 13.5±0.2, 14.0±0.2, 17.2±0.2, 19.6±0.2, 20.2±0.2, 20.8±0.2 and 21.0±0.2 (Cu Kα radiation, expressed in degrees 2 θ).

In some embodiments, cobicistat or Formula III can be adsorbed onto a solid carrier. In some embodiments, cobicistat can be adsorbed onto a solid carrier that is a plurality of silica particles. In some embodiments, cobicistat is adsorbed onto silicon dioxide particles (e.g., fumed silicon dioxide). In certain embodiments, where a percentage or weight amount for cobicistat or Formula III adsorbed onto a solid carrier (e.g. silica particles or silicon dioxide) is provided, the percentage or weight amount refer to cobicistat or Formula III plus the solid carrier.

The amount of cobicistat in a solid oral dosage form provided herein is generally between 60 mg and 240 mg, for instance between 140 mg and 160 mg, and more typically between 145 mg and 155 mg. In some embodiments, solid oral dosage forms containing 150 mg of cobicistat are provided.

The amount of cobicistat in a solid oral dosage form provided herein is generally between 60 mg and 350 mg, for instance between 140 mg and 160 mg, and more typically between 145 mg and 155 mg. In some embodiments, solid oral dosage forms containing 150 mg of cobicistat are provided. In some embodiments, solid oral dosage forms containing 275-300 mg of cobicistat adsorbed onto silicon dioxide are provided.

In some embodiments, up to about 60%±10% (w/w) of cobicistat, or a pharmaceutically acceptable salt, co-crystal, or solvate thereof, is loaded onto the silicon dioxide particles. In some embodiments, the weight percentage of the cobicistat, or a pharmaceutically acceptable salt, co-crystal, or solvate thereof, to the silicon dioxide particles is 20-30%±15%. In some embodiments, the weight percentage of the cobicistat, or a pharmaceutically acceptable salt, co-crystal, or solvate thereof, to the silicon dioxide particles is 45-50%±15%. In some embodiments, the weight percentage of the cobicistat, or a pharmaceutically acceptable salt, co-crystal, or solvate thereof, to the silicon dioxide particles is 47-56%±10%. In some embodiments, the (weight of the cobicistat, or a pharmaceutically acceptable salt, co-crystal, or solvate thereof) divided by the (weight of the silicon dioxide particles) in a composition is from about 0.2 to about 1.9. In some embodiments, the (weight of the cobicistat, or a pharmaceutically acceptable salt, co-crystal, or solvate thereof) divided by the (weight of the silicon dioxide particles) in a composition is from about 0.5 to about 1.5. In some embodiments, the (weight of the cobicistat, or a pharmaceutically acceptable salt, co-crystal, or solvate thereof) divided by the (weight of the silicon dioxide particles) in a composition is from about 0.8 to about 1.2. In some embodiments, the (weight of the cobicistat, or a pharmaceutically acceptable salt, co-crystal, or solvate thereof) divided by the (weight of the silicon dioxide particles) in a composition is about 1.0±0.5%.

### Darunavir

Darunavir is a HIV-1 protease inhibitor having the formula below (Formula IV) (see, e.g., U.S. Pat. No. 6,248,775):

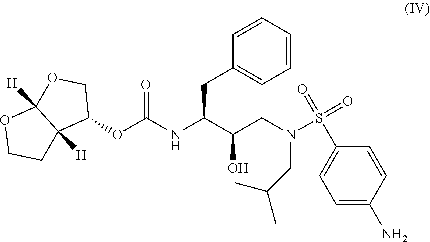 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

Darunavir refers to as [(1S,2R)-3-[[(4-aminophenyl)sulfonyl](2-methylpropyl)amino]-2-hydroxy-1-(phenylmethyl)propyl]-carbamic acid (3R,3aS,6aR)-hexahydrofuro[2,3-b]furan-3-yl ester monoethanolate. It is currently authorized as part of products such as PREZCOBIX® (darunavir 800 mg and cobicistat 150 mg) and PREZISTA® (darunavir 75 mg, 150 mg, 600 mg, and 800 mg).

Solid oral dosage forms disclosed herein include darunavir, optionally as a pharmaceutically acceptable salt or solvate thereof. darunavir can be present within an oral dosage form in solvated or unsolvated form, and references to “darunavir” include both of these forms. In certain embodiments, darunavir is present in the disclosed solid dosage formulations in an ethanolate form.

The amount of darunavir in a solid oral dosage form provided herein is generally between 320 mg and 1280 mg, for instance between 790 mg and 810 mg, and more typically between 795 mg and 805 mg. In some embodiments, solid oral dosage forms containing 800 mg of darunavir are provided. In some embodiments, solid oral dosage forms containing 400 mg to 800 mg of darunavir, or a pharmaceutically acceptable salt or solvate thereof, are provided. In some embodiments, solid oral dosage forms containing 600 mg to 800 mg of darunavir, or a pharmaceutically acceptable salt or solvate thereof, are provided.

The amount of darunavir in a solid oral dosage form provided herein is generally between 320 mg and 1280 mg, for instance between 600 mg to 900 mg, between 800 mg to 900 mg, between 790 mg and 810 mg, between 840 mg and 900 mg, between 795 mg and 805 mg, or between 850 mg and 890 mg. In some embodiments, solid oral dosage forms containing 800 mg of darunavir, ethanolate form are provided. In some embodiments, solid oral dosage forms containing 600 mg to 800 mg, or 870 mg, of darunavir, ethanolate form are provided. In some embodiments, solid oral dosage forms containing 867 mg of darunavir, ethanolate form, or a pharmaceutically acceptable salt or solvate thereof, are provided.

### Solid Oral Dosage Forms

In some embodiments, disclosed herein is a solid oral dosage form comprising:

(a) a compound of Formula I:

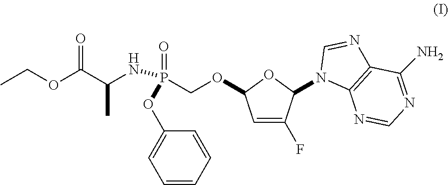 [SMILES: CCOC(=O)[C@H](C)N[P@](=O)(CO[C@@H]1C=C(F)[C@H](n2cnc3c(N)ncnc32)O1)Oc1ccccc1]

or a pharmaceutically acceptable salt thereof;

(b) a compound of Formula II:

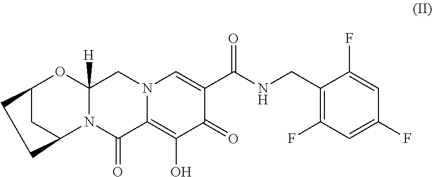 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof;

(c) a compound of Formula III:

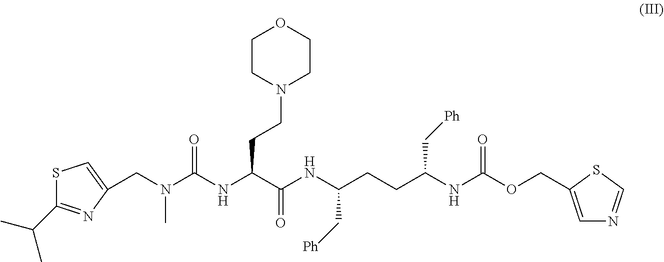 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt thereof; and

(d) a compound of Formula IV:

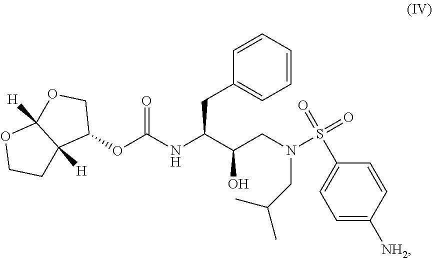 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

or a pharmaceutically acceptable salt or solvate thereof.

In some embodiments, the solid oral dosage form comprises 30 mg±60% of the compound of Formula I, or a pharmaceutically acceptable salt thereof; 50 mg±60% of the compound of Formula II, or a pharmaceutically acceptable salt thereof; 150 mg±60% of the compound of Formula III, or a pharmaceutically acceptable salt thereof; and 800 mg±60% of the compound of Formula IV, or a pharmaceutically acceptable salt or solvate thereof.

In some embodiments, the dosage form comprises 30 mg±60% of the compound of Formula I as a pharmaceutically acceptable salt thereof; 50 mg±60% of the compound of Formula II as a pharmaceutically acceptable salt thereof; 150 mg±60% of the compound of Formula III; and 800 mg±60% of the compound of Formula IV as a pharmaceutically acceptable salt or solvate thereof.

In some embodiments, the dosage form comprises 30 mg±60% of the compound of Formula I as a pharmaceutically acceptable salt thereof; 50 mg±60% of the compound of Formula II as a pharmaceutically acceptable salt thereof; 150 mg±60% of cobicistat; and 800 mg±60% of darunavir.

In some embodiments, the solid oral dosage form further comprises a plurality of silica particles. In some embodiments, the compound of Formula III is adsorbed onto the silica particles.

The solid oral dosage forms disclosed herein are intended for pharmaceutical use in human subjects. Accordingly, they must be of an appropriate size and weight for oral human administration (e.g. they should have a total weight of less than about 1.5 g, e.g., less than about 1.0 g), in addition to being therapeutically efficacious.

In some embodiments, the solid oral dosage form is a tablet comprising: a coating and 30 mg±60% of a compound of Formula I:

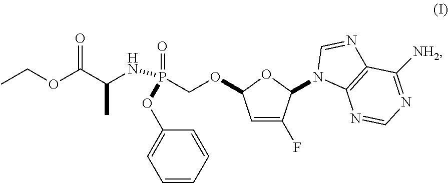 [SMILES: CCOC(=O)[C@H](C)N[P@](=O)(CO[C@@H]1C=C(F)[C@H](n2cnc3c(N)ncnc32)O1)Oc1ccccc1]

or a pharmaceutically acceptable salt thereof, 50 mg±60% of the compound of Formula II, or a pharmaceutically acceptable salt thereof, (c) 150 mg±60% of the compound of Formula III, or a pharmaceutically acceptable salt thereof, and (d) 800 mg±60% of the compound of Formula IV, or a pharmaceutically acceptable salt or solvate thereof.

In some embodiments, disclosed herein is a tablet comprising:

(a) 30 mg±60% of a compound of Formula Ia:

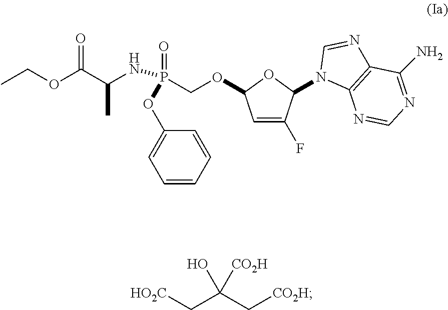 [SMILES: CCOC(=O)[C@H](C)N[P@](=O)(CO[C@@H]1C=C(F)[C@H](n2cnc3c(N)ncnc32)O1)Oc1ccccc1 ; O=C(O)CC(O)(CC(=O)O)C(=O)O]

(b) 50 mg±60% a compound of Formula IIa:

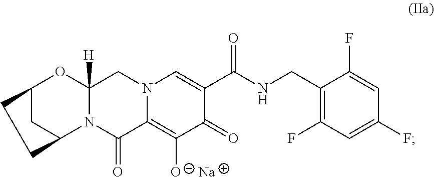 [SMILES: O=C(NCc1c(F)cc(F)cc1F)c1cn2c(c([O-])c1=O)C(=O)N1[C@H]3CC[C@H](C3)O[C@@H]1C2 ; [Na+]]

(c)150 mg±60% a compound of Formula III:

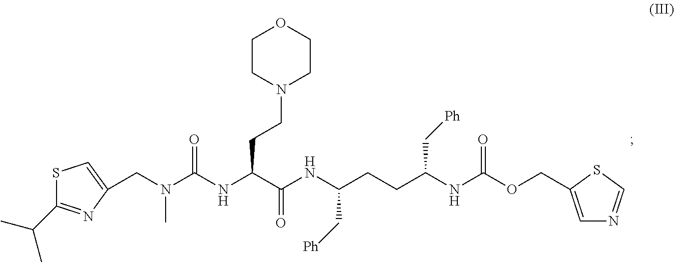 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

and

(d) 800 mg ±60% of the compound of Formula IV:

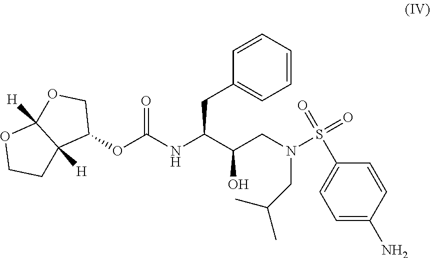 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

or a pharmaceutically acceptable salt or solvate thereof.

In some embodiments, disclosed herein is a solid oral dosage form comprising:

about 0.5% to about 2.5% w/w of a compound of Formula I:

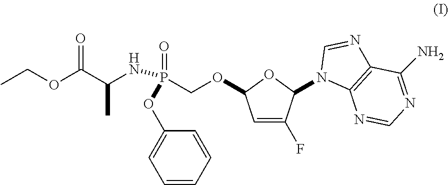 [SMILES: CCOC(=O)[C@H](C)N[P@](=O)(CO[C@@H]1C=C(F)[C@H](n2cnc3c(N)ncnc32)O1)Oc1ccccc1]

or a pharmaceutically acceptable salt thereof; about 2% to about 6% w/w of a compound of Formula II:

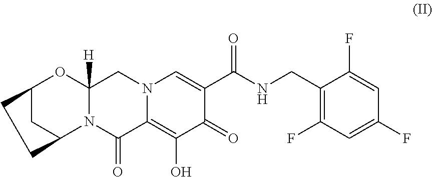 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof;

about 7% to about 12% w/w a compound of Formula III:

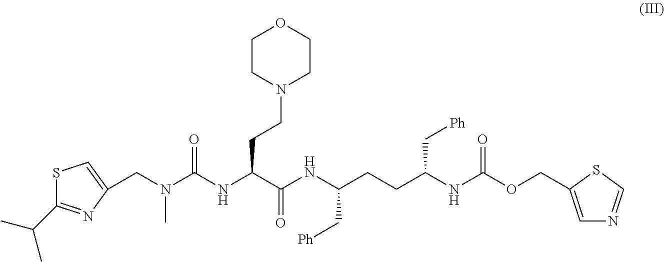 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt thereof; and about 40% to about 75% w/w of a comnound of Formula IV:

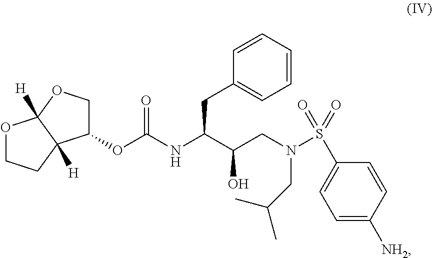 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

or a pharmaceutically acceptable salt or solvate thereof.

In some embodiments, disclosed herein is a solid oral dosage form comprising:
(a) about 10 mg to about 30 mg of a compound of Formula I, or a pharmaceutically acceptable salt thereof;
(b) about 25 mg to about 75 mg of a compound of Formula II, or a pharmaceutically acceptable salt thereof;
(c) about 150 mg to about 350 mg of a compound of Formula III, or a pharmaceutically acceptable salt thereof;
(d) about 600 mg to about 1000 mg of a compound of Formula IV, or a pharmaceutically acceptable salt or solvate thereof;
(e) about 100 mg to about 175 mg of microcrystalline cellulose; about 65 mg to about 105 mg of crospovidone; and
(f) about 10 mg to about 30 mg of magnesium stearate.

In some embodiments, disclosed herein is a solid oral dosage form comprising:
(a) about 1% to about 3% w/w of a compound of Formula I:

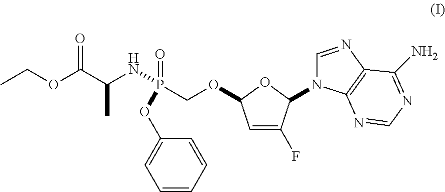

or a pharmaceutically acceptable salt thereof;
(b) about 1% to about 5% w/w of a compound of Formula II:

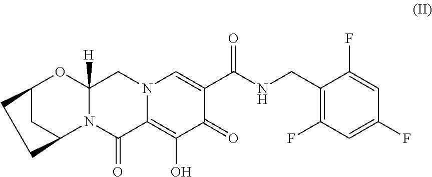 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof;
(c) about 5% to about 20% w/w a compound of Formula III:

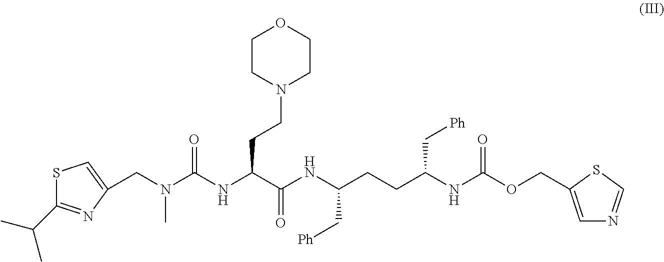 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt thereof; and
(d) about 50% to about 70% w/w of a compound of Formula IV:

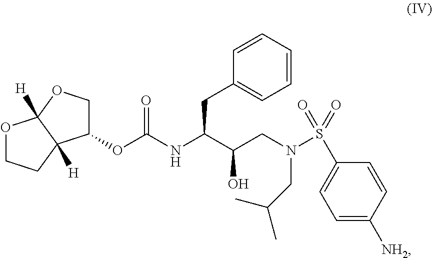 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

or a pharmaceutically acceptable salt or solvate thereof

In some embodiment, disclosed herein is a solid oral dosage form comprising:
(a) about 10 mg to about 15 mg of a compound of Formula I, or a pharmaceutically acceptable salt thereof;
(b) about 40 mg to about 60 mg of a compound of Formula II, or a pharmaceutically acceptable salt thereof;
(c) about 230 mg to about 320 mg of a compound of Formula III, or a pharmaceutically acceptable salt thereof, and silicon dioxide particles;
(d) about 700 mg to about 900 mg of a compound of Formula IV, or a pharmaceutically acceptable salt or solvate thereof;
(e) about 110 mg to about 165 mg of microcrystalline cellulose;
(f) about 75 mg to about 100 mg of crospovidone; and
(g) about 15 mg to about 25 mg of magnesium stearate.

In some embodiments, disclosed herein is a solid oral dosage form comprising:
(a) about 20 mg to about 40 mg of a compound of Formula I, or a pharmaceutically acceptable salt thereof;
(b) about 15 mg to about 50 mg of a compound of Formula II, or a pharmaceutically acceptable salt thereof;
(c) about 100 mg to about 200 mg of a compound of Formula III, or a pharmaceutically acceptable salt thereof;
(d) about 600 mg to about 1000 mg of a compound of Formula IV, or a pharmaceutically acceptable salt or solvate thereof;
(e) about 100 mg to about 175 mg of microcrystalline cellulose;
(f) about 65 mg to about 105 mg of crospovidone; and
(g) about 5 mg to about 20 mg of magnesium stearate.

In some embodiments, disclosed herein is a solid oral dosage form comprising:

(a) about 2% to about 5% w/w of a compound of Formula I:

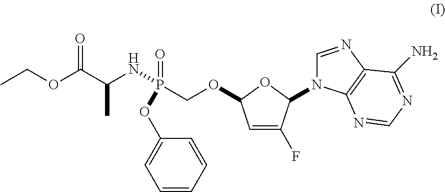

or a pharmaceutically acceptable salt thereof;

(b) about 1% to about 5% w/w of a compound of Formula II:

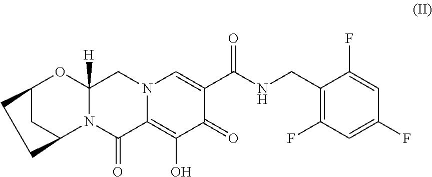 [SMILES: O=C(NCc1c(F)cc(F)cc1F)c1cn2c(c(O)c1=O)C(=O)N1[C@H]3CC[C@H](C3)O[C@@H]1C2]

or a pharmaceutically acceptable salt thereof;

(c) about 15% to about 25% w/w a compound of Formula III on SiO₂:

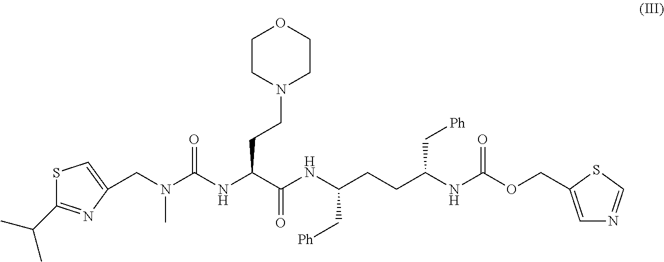 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt thereof; and

(d) about 50% to about 70% w/w of a compound of Formula IV:

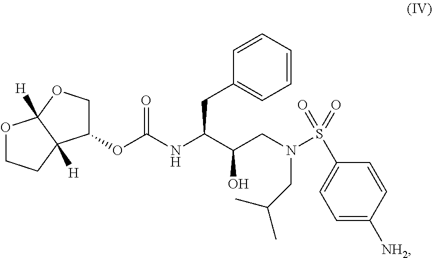 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

or a pharmaceutically acceptable salt or solvate thereof

In some embodiments, disclosed herein is a solid oral dosage form comprising:
(a) about 40 mg to about 80 mg of a compound of Formula I, or a pharmaceutically acceptable salt thereof;
(b) about 15 mg to about 50 mg of a compound of Formula II, or a pharmaceutically acceptable salt thereof;
(c) about 100 mg to about 300 mg of a compound of Formula III, or a pharmaceutically acceptable salt thereof;
(d) about 600 mg to about 1000 mg of a compound of Formula IV, or a pharmaceutically acceptable salt or solvate thereof;
(e) about 75 mg to about 125 mg of microcrystalline cellulose;
(f) about 60 mg to about 110 mg of crospovidone; and
(g) about 5 mg to about 20 mg of magnesium stearate.

In some embodiments, the fixed dose combination formulation tablet disclosed herein includes the compound of Formula III adsorbed onto a solid carrier. In certain embodiments, the solid carrier includes silica particles. In certain embodiments, the solid carrier is silicon dioxide. In certain embodiments of fixed dose combination formulation tablet, the amount of silicon dioxide is approximately 140 mg. In certain embodiments of the fixed dose combination formulation tablet, the amount of silicon dioxide is approximately 10% by weight of the solid dosage form.

In some embodiments, disclosed herein is a solid oral dosage form comprising:
(a) about 40 mg to about 80 mg of a compound of Formula I, or a pharmaceutically acceptable salt thereof;
(b) about 15 mg to about 50 mg of a compound of Formula II, or a pharmaceutically acceptable salt thereof;
(c) about 200 mg to about 400 mg of a compound of Formula III on SiO₂, or a pharmaceutically acceptable salt thereof;
(d) about 600 mg to about 1000 mg of a compound of Formula IV, or a pharmaceutically acceptable salt or solvate thereof;
(e) about 75 mg to about 125 mg of microcrystalline cellulose;
(f) about 60 mg to about 110 mg of crospovidone; and
(g) about 5 mg to about 20 mg of magnesium stearate.

In certain embodiments, in the solid oral dosage forms disclosed herein, the compound of Formula I is in its free base form, Formula II is an acid in its free form, and Formula IV is in its ethanolate form. In certain embodiments, the solid oral dosage form contains 30 mg of the compound of Formula I, 31.5 mg of the compound of Formula II, or a pharmaceutically acceptable salt thereof, 150 mg of the compound of Formula III, and 869 mg of the compound of Formula IV, ethanolate form. In certain embodiments, the solid oral dosage form contains 30 mg of the compound of Formula I, 31.5 mg of the compound of Formula II, ethanolate form, 150 mg of the compound of Formula III on approximately 140 mg SiO₂, and 869 mg of the compound of Formula IV. In certain embodiments, the solid oral dosage form contains 60 mg of the compound of Formula I, 31.5 mg of the compound of Formula II, ethanolate form, 150 mg of the compound of Formula III, and 869 mg of the compound of Formula IV. In certain embodiments, the solid oral dosage form contains 60 mg of the compound of Formula I, 31.5 mg of the compound of Formula II, ethanolate form, 150 mg of the compound of Formula III on approximately 140 mg SiO₂, and 869 mg of the compound of Formula IV.

The solid oral dosage forms disclosed herein will typically be in the form of a fixed dose combination tablet. This is because the inventors have found that the use of fixed dose combination tablets may assist in optimizing the pharmacokinetic properties of the active ingredients, particularly the total exposure of the compound of Formula II or a pharmaceutically acceptable salt thereof, as measured by area under the curve (AUC) and Cₘₐₓ. In some embodiments, the solid oral dosage forms disclosed herein are in the form of a multilayer tablet. In some embodiments, the solid oral dosage forms disclosed herein are in the form of a monolayer tablet. In some embodiments, the use of a fixed dose combinations, e.g., multilayer tablets, may affect the dissolution profile of one or more of the active ingredients within the dosage form, and is therefore likely to have an impact on the in vivo pharmacokinetics of the dosage form.

In some embodiments, disclosed herein is a multilayer tablet (e.g., bilayer tablet, trilayer tablet) comprising (a) the compound of Formula I or a pharmaceutically acceptable salt thereof, (b) the compound of Formula II, or a pharmaceutically acceptable salt thereof, (c) cobicistat, or a pharmaceutically acceptable salt thereof, and (d) darunavir, or a pharmaceutically acceptable salt or solvate thereof. Typically, each layer contains at least one of (a), (b), (c), and (d). For instance, in some embodiments, the tablet comprises a first layer comprising (a) the compound of Formula I or a pharmaceutically acceptable salt thereof, and (c) cobicistat or a pharmaceutically acceptable salt thereof. In some embodiments, the tablet comprises a first layer comprising (b) the compound of Formula II or a pharmaceutically acceptable salt thereof, and (c) cobicistat or a pharmaceutically acceptable salt thereof. In some embodiments, the tablet comprises a first layer comprising (a) the compound of Formula I or a pharmaceutically acceptable salt thereof, (b) the compound of Formula II or a pharmaceutically acceptable salt thereof, and (c) cobicistat or a pharmaceutically acceptable salt thereof.

The solid oral dosage forms disclosed herein are intended for pharmaceutical use in human subjects. Accordingly, they must be of an appropriate size and weight for oral human administration (e.g. they should have a total weight of less than about 1.6 g, less than about 1.5 g, less than about 1.4 g or less than about 1.3 g), in addition to being therapeutically efficacious.

In some embodiments, the tablet disclosed herein is formulated for once a day dosing.

Unless otherwise specified, the terms “first layer”, “second layer”, “third layer” and so forth do not specify a particular order or orientation of the multilayer tablet formulations disclosed herein. Rather, these terms are used to distinguish the sections of the composition from each other and to specify the characteristics or components of each section or compartment. The first layer may be synthesized first or may be synthesized second. The first layer may be on the bottom or may be on the top or may be on a side. The term “first layer” is not limiting as to order and orientation.

The tablets disclosed herein are typically immediate release tablets. In one embodiment, a tablet is provided which releases at least 50% of the compound of Formula II or a pharmaceutically acceptable salt thereof in about 20 minutes, measured using USP apparatus II, in 333 mL of fasted state simulated intestinal fluid, pH 6.5, at 37° C. and paddle speed of 100 rpm. In certain embodiments, the tablets disclosed herein release at least 60% of the compound of Formula II or a pharmaceutically acceptable salt thereof in 20 minutes, measured using USP apparatus II, in 333 mL of 50 mM fasted state simulated intestinal fluid, at 37° C. and paddle speed of 100 rpm. In some embodiments, a tablet that releases at least 70% of the compound of Formula II in 60 minutes is provided, measured using USP Apparatus II, in 333 mL of fasted state simulated intestinal fluid at 37° C. and paddle speed of 100 rpm.

The disclosed solid oral dosage forms have high levels of drug loading, i.e., a high percentage of active pharmaceutical ingredient relative to the total tablet weight. The development of solid oral dosage forms having multiple active ingredients with relatively high drug loading challenges due to the potential for the active ingredients to interacts with each other, for example chemically or physically or both. These challenges are amplified when the active ingredients are present in divergent amounts, i.e., where there is a relatively small amount of one or two ingredients relative to the amount of the other active ingredients in the dosage form. For example, in one formulation of the solid oral dosage forms disclosed herein, the compound of Formula I is typically present in the dosage form at less than about 5% w/w or even at less than 3% w/w, or less than about 2.5% w/w or even at less than 1% w/w, while the compound of Formula IV is present in an amount at least about 40% w/w or about 50-60% w/w. Moreover, the compound of Formula I, in particular, is susceptible to hydrolysis and exhibits maximum stability at pH 5, while the compound of Formula III is known to be hygroscopic. Thus, there exists the potential for chemical reactions or physical interactions between the active ingredients. Despite these challenges, it has been found that the solid oral dosage forms disclosed herein are chemically stable, for example at accelerated conditions (e.g., 40° C., 75% RH and/or 25° C., 60% RH).

It has also been found that the powder blends of the solid oral dosage forms disclosed herein are highly compressible, exhibiting relatively high tensile strength (e.g., greater than 1.6 MPa or 1.8 MPa) as well as having other favorable manufacturing characteristics.

In one embodiment, the solid oral dosage forms disclosed herein include at least about 70% or at least about 80% or at least about 90% active pharmaceutical ingredients. In one embodiment, the solid oral dosage forms disclosed herein include about 70% to about 85%, about 70% to about 75%, about 75% to about 85%, or about 80% to about 85% active pharmaceutical ingredients of the total tablet weight.

Tablets disclosed herein will generally have a hardness within the range 14-20 kP, and, in certain specific embodiments, have a hardness of 17 kP. In some embodiments, tablets disclosed herein will generally have a hardness of at least about 25 or within a range of about 25-35 kP, and, in certain specific embodiments, have a hardness of about 30 kP. Hardness can conveniently be assessed by driving an anvil to compress a tablet at a constant loading rate until it fractures, operating in accordance with USP<1217>(using e.g. a TBH 220, ERWEKA GmbH, Heusenstamm Germany hardness tester).

Tablets disclosed herein will generally have a friability of<1% by weight. Friability can be assessed according to USP<1216>.

The core of a tablet provided herein may have a hardness of at least about 25 kP, and a friability of<1% by weight or a hardness of at least about 30 kP, and a friability of<1% by weight.

Tablets will typically include one or more excipients. Excipients should be compatible with the other ingredients of the formulation and physiologically innocuous to the recipient thereof. Examples of suitable excipients are well known to the person skilled in the art of tablet formulation and may be found e.g. in Handbook of Pharmaceutical Excipients (eds. Rowe, Sheskey & Quinn), 6 th edition 2009. As used herein the term “excipients” is intended to refer to inter alia basifying agents, solubilisers, glidants, fillers, binders, lubricant, diluents, preservatives, surface active agents, dispersing agents and the like. The term also includes agents such as sweetening agents, flavoring agents, coloring agents, preserving agents, and coating agents. Such components will generally be present in admixture within the tablet.

Examples of solubilisers include, but are not limited to, ionic surfactants (including both ionic and non-ionic surfactants) such as sodium lauryl sulphate, cetyltrimethylammonium bromide, polysorbates (such as polysorbate 20 or 80), poloxamers (such as poloxamer 188 or 207), and macrogols. In a particular embodiment, a tablet that comprises the compound of Formula I or a pharmaceutically acceptable salt thereof, includes a polysorbate, in particular polysorbate 20. In certain specific embodiments, the amount of polysorbate 20 in a tablet disclosed herein is less than about 5 mg, such as less than about 1 mg, or about 0.5 mg.

Examples of lubricants, glidants and flow aids include, but are not limited to, magnesium stearate, calcium stearate, stearic acid, hydrogenated vegetable oil, glyceryl palmitostearate, glyceryl behenate, sodium stearyl fumarate, colloidal silicon dioxide, and talc. The amount of lubricant in a tablet is generally between about 0.5-5% by weight. In certain embodiments, the amount of lubricant in a tablet is about 1.5% by weight. In certain embodiments, the tablet includes less than about 10 mg magnesium stearate, or less than about 7.5 mg magnesium stearate.

Examples of disintegrants include, but are not limited to, starches, celluloses, cross-linked PVP (crospovidone), sodium starch glycolate, croscarmellose sodium, etc. In certain embodiments, the tablets disclosed herein include croscarmellose sodium. In certain embodiments, the tablet includes less than about 50 croscarmellose sodium, or less than about 25 mg croscarmellose sodium.

Examples of fillers (also known as bulking agents or diluents) include, but are not limited to, starches, maltodextrins, polyols (such as lactose, lactose anhydrous, lactose monohydrate, etc.), and celluloses. In certain embodiments, tablets provided herein may microcrystalline cellulose. In certain embodiments, tablets provided herein include less than about 300 mg microcrystalline cellulose, in particular less than about 250 mg microcrystalline cellulose, and/or less than about 225 mg microcrystalline cellulose.

Examples of binders include, but are not limited to, cross-linked PVP, HPMC, sucrose, starches, etc.

In certain embodiments, tablets provided herein are uncoated. In certain other embodiments, tablets provided herein are coated (in which case they include a coating). Although uncoated tablets may be used, it is more usual in the clinical setting to provide a coated tablet, in which case a conventional non-enteric coating may be used. Film coatings are known in the art and can be composed of hydrophilic polymer materials, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though in certain embodiments the water soluble material included in the film coating of the embodiments disclosed herein includes a single polymer material, in certain other embodiments it is formed using a mixture of more than one polymer. In certain embodiments, the coating is red, gray, blue, yellow or brown. Suitable coatings include, but are not limited to, polymeric film coatings such as those comprising polyvinyl alcohol e.g. ‘Opadry® II’ (which includes part-hydrolysed PVA, titanium dioxide, polyethylene glycol (PEG, e.g., macrogol 3350) and talc, with optional coloring such as iron oxide (e.g., iron oxide red, iron oxide black, iron oxide yellow), indigo carmine, or FD&C yellow #6). The amount of coating is generally between about 2-4% of the core's weight, and in certain specific embodiments, about 3%. Unless specifically stated otherwise, where the dosage form is coated, it is to be understood that a reference to % weight of the tablet means that of the total tablet, i.e. including the coating.

### Manufacturing Methods

In general, tableting methods are well known in the art of pharmacy. Techniques and formulations generally are found in Remington Pharmaceutical Sciences (Mack Publishing Co., Easton, Pa.), which is hereby incorporated by reference herein in its entirety.

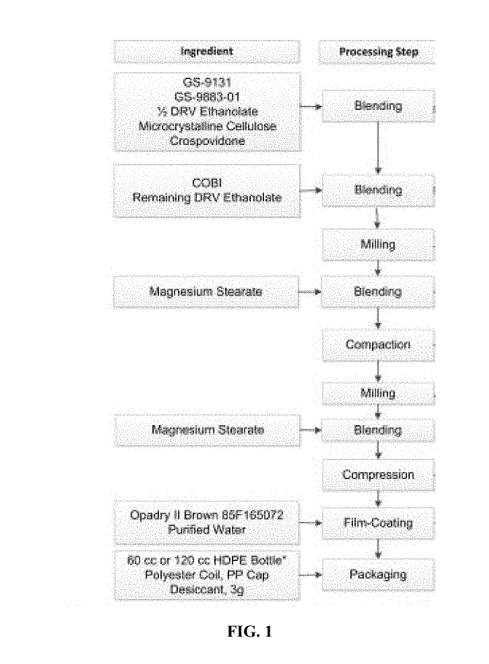

For example, methods for obtaining the compounds described herein will be apparent to those of ordinary skill in the art, with suitable procedures being described, for example, in U.S. Pat. Nos. 7,871,991, 8,987,437, 9,216,996, 8,497,396, and 7,772,411, and in the references cited herein. An example of the manufacturing process is shown in FIG. 1 and described in Example 8, and is not intended to be limiting in the manufacturing of the solid dosage formulations described herein. Other manufacturing processes known in the pharmaceutical manufacturing processes field may be effective in arriving at the desired solid form dosages described herein.

Methods for producing the compositions and dosage forms (in particular tablets) disclosed herein are also provided. For example, a first layer comprising a compound selected from: (a) the compound of Formula I, or a pharmaceutically acceptable salt thereof, (b) the compound of Formula II, or a pharmaceutically acceptable salt thereof, (c) cobicistat, or a pharmaceutically acceptable salt thereof, and (d) darunavir, or a pharmaceutically acceptable salt or solvate thereof, may be formed by compression and subsequently a second layer may be compressed onto the first layer. In certain embodiments, the choice of layer order in the tableting of multilayer tablets may have an impact on the properties of the tablets (e.g. the adhesion of the layers within the tablet).

In certain embodiments, the methods will include a step of coating the tablet cores after compression, e.g. with a film coating as described above.

A tablet can be made by compression or molding, optionally with one or more excipients. Compressed tablets may be prepared by compressing in a suitable machine the active ingredient in a free-flowing form such as a powder or granules, optionally mixed with excipients.

In certain embodiments, the solid oral dosage forms including the four active ingredients have a total weight of less than about 1.6 g or less than about 1.5 g. The provision of a relatively small dosage form (in particular a tablet) represents a clinical advantage because it may be expected to increase patient convenience and thus compliance as compared to larger dosage forms which are more burdensome for patients to swallow. In specific embodiments, the solid oral dosage form disclosed herein has a total weight of between 1400 mg and 1550 mg. In certain embodiments, the solid oral dosage form disclosed herein has a total weight of between 1440 mg and 1500 mg.

In certain embodiments, the presently disclosed dosage forms include less than about 600 mg of excipients or less than about 300 mg of excipients. For example, solid oral dosage forms disclosed herein comprise between 275 mg and 450 mg of excipients. In some embodiments, solid oral dosage forms disclosed herein comprise between 275 mg and 300 mg of excipients. In some embodiments, solid oral dosage forms disclosed herein comprise between 375 mg and 425 mg of excipients. In such embodiments, the dosage forms comprise as active ingredients (a) about 10 mg to about 30 mg of the compound of Formula I or pharmaceutically acceptable salt thereof, (b) about 25 mg to about 75 mg of the compound of Formula II or pharmaceutically acceptable salt thereof, (c) about 150 mg to about 300 mg of the compound of Formula III or a pharmaceutically acceptable salt thereof, (d) about 600 mg to about 100 mg of the compound of Formula IV or a pharmaceutically acceptable salt or solvate thereof. In other such embodiments, the dosage forms comprise as active ingredients (a) about 20 mg to about 40 mg of the compound of Formula I or pharmaceutically acceptable salt thereof, (b) about 25 mg to about 75 mg of the compound of Formula II or pharmaceutically acceptable salt thereof, (c) about 150 mg to about 300 mg of the compound of Formula III or a pharmaceutically acceptable salt thereof, (d) about 600 mg to about 100 mg of the compound of Formula IV or a pharmaceutically acceptable salt or solvate thereof. In certain embodiments, the compound of Formula III or a pharmaceutically acceptable salt thereof includes solid carrier particles (e.g., the compound of Formula III is adsorbed onto silicon dioxide particles). In such embodiments, the dosage forms comprise as active ingredients (a) 10 mg to about 30 mg of the compound of Formula I or pharmaceutically acceptable salt thereof, (b) about 25 mg to about 75 mg of the compound of Formula II or pharmaceutically acceptable salt thereof, (c) about 250 to about 300 mg of the compound of Formula III or a pharmaceutically acceptable salt thereof, (d) about 600 mg to about 1000 mg of the compound of Formula IV or a pharmaceutically acceptable salt or solvate thereof. In other such embodiments, the dosage forms comprise as active ingredients (a) about 55 mg to about 75 mg of the compound of Formula I or pharmaceutically acceptable salt thereof, (b) about 25 mg to about 75 mg of the compound of Formula II or pharmaceutically acceptable salt thereof, (c) about 250 to about 300 mg of the compound of Formula III or a pharmaceutically acceptable salt thereof, (d) about 600 mg to about 1000 mg of the compound of Formula IV or a pharmaceutically acceptable salt or solvate thereof.

In one embodiment, a solid oral dosage forms includes (a) about 10 mg of the compound of Formula I or pharmaceutically acceptable salt thereof, (b) about 50 mg to about 55 mg of the compound of Formula II or pharmaceutically acceptable salt thereof, (c) about 150 mg of the compound of Formula III or a pharmaceutically acceptable salt thereof, (d) about 800 mg to about 875 mg of the compound of Formula IV or a pharmaceutically acceptable salt or solvate thereof; and about 375 mg to about 425 mg excipients.

In one embodiment, a solid oral dosage forms includes (a) about 10 mg of the compound of Formula I or pharmaceutically acceptable salt thereof, (b) about 50 mg to about 55 mg of the compound of Formula II or pharmaceutically acceptable salt thereof, (c) about 275 mg to about 300 mg of the compound of Formula III or a pharmaceutically acceptable salt thereof adsorbed onto a solid carrier, (d) about 800 mg to about 875 mg of the compound of Formula IV or a pharmaceutically acceptable salt or solvate thereof, and about 250 mg to about 300 mg excipients.

In an embodiment, a tablet is provided, comprising (a) about 10 mg of the compound of Formula I or pharmaceutically acceptable salt thereof, (b) about 50 mg to about 55 mg of the compound of Formula II or pharmaceutically acceptable salt thereof, (c) about 275 mg to about 300 mg of the compound of Formula III or a pharmaceutically acceptable salt thereof adsorbed onto a solid carrier, and (d) about 800 mg to about 875 mg of the compound of Formula IV or a pharmaceutically acceptable salt or solvate thereof. In one embodiment, the tablet is a monolayer tablet.

In one embodiment, a solid oral dosage forms includes (a) about 30 mg of the compound of Formula I or pharmaceutically acceptable salt thereof, (b) about 50 mg to about 55 mg of the compound of Formula II or pharmaceutically acceptable salt thereof, (c) about 150 mg of the compound of Formula III or a pharmaceutically acceptable salt thereof, (d) about 800 mg to about 875 mg of the compound of Formula IV or a pharmaceutically acceptable salt or solvate thereof; and about 375 mg to about 425 mg excipients.

In one embodiment, a solid oral dosage forms includes (a) about 30 mg of the compound of Formula I or pharmaceutically acceptable salt thereof, (b) about 50 mg to about 55 mg of the compound of Formula II or pharmaceutically acceptable salt thereof, (c) about 275 mg to about 300 mg of the compound of Formula III or a pharmaceutically acceptable salt thereof adsorbed onto a solid carrier, (d) about 800 mg to about 875 mg of the compound of Formula IV or a pharmaceutically acceptable salt or solvate thereof; and about 250 mg to about 300 mg excipients.

In an embodiment, a tablet is provided, comprising (a) about 30 mg of the compound of Formula I or pharmaceutically acceptable salt thereof, (b) about 50 mg to about 55 mg of the compound of Formula II or pharmaceutically acceptable salt thereof, (c) about 275 mg to about 300 mg of the compound of Formula III or a pharmaceutically acceptable salt thereof adsorbed onto a solid carrier, and (d) about 800 mg to about 875 mg of the compound of Formula IV or a pharmaceutically acceptable salt or solvate thereof.

In one embodiment, a solid oral dosage forms includes (a) about 60 mg of the compound of Formula I or pharmaceutically acceptable salt thereof, (b) about 50 mg to about 55 mg of the compound of Formula II or pharmaceutically acceptable salt thereof, (c) about 150 mg of the compound of Formula III or a pharmaceutically acceptable salt thereof, (d) about 800 mg to about 875 mg of the compound of Formula IV or a pharmaceutically acceptable salt or solvate thereof; and about 100 mg to about 400 mg excipients.

In one embodiment, a solid oral dosage forms includes (a) about 60 mg of the compound of Formula I or pharmaceutically acceptable salt thereof, (b) about 50 mg to about 55 mg of the compound of Formula II or pharmaceutically acceptable salt thereof, (c) about 275 mg to about 300 mg of the compound of Formula III or a pharmaceutically acceptable salt thereof adsorbed onto a solid carrier, (d) about 800 mg to about 875 mg of the compound of Formula IV or a pharmaceutically acceptable salt or solvate thereof; and about 125 mg to about 300 mg excipients.

In an embodiment, a tablet is provided, comprising (a) about 60 mg of the compound of Formula I or pharmaceutically acceptable salt thereof, (b) about 50 mg to about 55 mg of the compound of Formula II or pharmaceutically acceptable salt thereof, (c) about 275 mg to about 300 mg of the compound of Formula III or a pharmaceutically acceptable salt thereof adsorbed onto a solid carrier, and (d) about 800 mg to about 875 mg of the compound of Formula IV or a pharmaceutically acceptable salt or solvate thereof; and about 150 mg to about 225 mg excipients.

In one embodiment, a solid oral dosage forms includes (a) about 30 mg of the compound of Formula I or pharmaceutically acceptable salt thereof, (b) about 30 mg of the compound of Formula II or pharmaceutically acceptable salt thereof, (c) about 150 mg of the compound of Formula III or a pharmaceutically acceptable salt thereof, (d) about 800 mg to about 875 mg of the compound of Formula IV or a pharmaceutically acceptable salt or solvate thereof; and about 125 mg to about 350 mg excipients.

In one embodiment, a solid oral dosage forms includes (a) about 30 mg of the compound of Formula I or pharmaceutically acceptable salt thereof, (b) about 30 mg of the compound of Formula II or pharmaceutically acceptable salt thereof, (c) about 275 mg to about 300 mg of the compound of Formula III or a pharmaceutically acceptable salt thereof adsorbed onto a solid carrier, (d) about 800 mg to about 875 mg of the compound of Formula IV or a pharmaceutically acceptable salt or solvate thereof; and about 250 mg to about 325 mg excipients.

In an embodiment, a tablet is provided, comprising (a) about 30 mg of the compound of Formula I or pharmaceutically acceptable salt thereof, (b) about 30 mg of the compound of Formula II or pharmaceutically acceptable salt thereof, (c) about 275 mg to about 300 mg of the compound of Formula III or a pharmaceutically acceptable salt thereof adsorbed onto a solid carrier, and (d) about 800 mg to about 875 mg of the compound of Formula IV or a pharmaceutically acceptable salt or solvate thereof.

In one embodiment, a solid oral dosage forms includes (a) about 60 mg of the compound of Formula I or pharmaceutically acceptable salt thereof, (b) about 30 mg of the compound of Formula II or pharmaceutically acceptable salt thereof, (c) about 150 mg of the compound of Formula III or a pharmaceutically acceptable salt thereof, (d) about 800 mg to about 875 mg of the compound of Formula IV or a pharmaceutically acceptable salt or solvate thereof; and about 225 mg to about 300 mg excipients.

In one embodiment, a solid oral dosage forms includes (a) about 60 mg of the compound of Formula I or pharmaceutically acceptable salt thereof, (b) about 30 mg of the compound of Formula II or pharmaceutically acceptable salt thereof, (c) about 275 mg to about 300 mg of the compound of Formula III or a pharmaceutically acceptable salt thereof adsorbed onto a solid carrier, (d) about 800 mg to about 875 mg of the compound of Formula IV or a pharmaceutically acceptable salt or solvate thereof; and about 225 mg to about 285 mg excipients.

In an embodiment, a tablet is provided, comprising (a) about 60 mg of the compound of Formula I or pharmaceutically acceptable salt thereof, (b) about 30 mg of the compound of Formula II or pharmaceutically acceptable salt thereof, (c) about 275 mg to about 300 mg of the compound of Formula III or a pharmaceutically acceptable salt thereof adsorbed onto a solid carrier, and (d) about 800 mg to about 875 mg of the compound of Formula IV or a pharmaceutically acceptable salt or solvate thereof; and about 230 mg to about 280 mg excipients.

In one embodiment, the tablet is a monolayer tablet. In other embodiments, the tablet is a bilayer or multilayer tablet.

In one embodiment, the tablet disclosed herein comprises one or more excipients, for example one or more diluents, disintegrants, binders, or lubricants.

In one embodiment, a tablet comprises microcrystalline cellulose, crospovidone, and magnesium stearate.

In one embodiment a tablet is provided wherein less than about 5 weight percent of the tablet is the compound of Formula I or a pharmaceutically acceptable salt thereof. In one embodiment a tablet is provided wherein less than about 1 weight percent of the tablet is the compound of Formula I or a pharmaceutically acceptable salt thereof. In one embodiment a tablet is provided wherein less than about 0.75 weight percent of the tablet is the compound of Formula I or a pharmaceutically acceptable salt thereof. In one embodiment a tablet is provided wherein about 0.65 to about 5 weight percent, or about 0.65 to about 2 weight percent, or about 0.65 to about 1 weight percent of the layer is the compound of Formula I or a pharmaceutically acceptable salt thereof.

In one embodiment a tablet is provided wherein less than about 10 weight percent of the tablet is the compound of Formula I or a pharmaceutically acceptable salt thereof. In one embodiment a tablet is provided wherein less than about 5 weight percent of the tablet is the compound of Formula I or a pharmaceutically acceptable salt thereof. In one embodiment a tablet is provided wherein less than about 3 weight percent of the tablet is the compound of Formula I or a pharmaceutically acceptable salt thereof. In one embodiment a tablet is provided wherein about 1 to about 2 weight percent, or about 2 to about 4 weight percent, or about 4 to about 5 weight percent of the layer is the compound of Formula I or a pharmaceutically acceptable salt thereof.

In one embodiment, the dosage form comprises:

| Ingredient | % (w/w) |
|---|---|
| The compound of Formula I or a salt thereof | 0.5-2.5 |
| The compound of Formula II or a salt thereof | 2-6 |
| The compound of Formula III or a salt thereof | 10-30 |
| The compound of Formula IV or a salt or solvate thereof | 40-75 |
| Microcrystalline cellulose | 5-15 |
| Crospovidone | 4-8 |
| Magnesium stearate | 1-2 |

In one embodiment, the dosage form comprises:

| Ingredient | % (w/w) |
|---|---|
| The compound of Formula I or a salt thereof | 0.5-1 |
| The compound of Formula II or a salt thereof | 2.5-4.5 |
| The compound of Formula III or a salt thereof | 15-25 |
| The compound of Formula IV or a salt or solvate thereof | 50-70 |
| Microcrystalline cellulose | 7-13 |
| Crospovidone | 5-7 |
| Magnesium stearate | 1.2-1.8 |

In one embodiment, the dosage form comprises:

| Ingredient | % (w/w) |
|---|---|
| The compound of Formula I or a salt thereof | 0.6-0.8 |
| The compound of Formula II or a salt thereof | 4-5 |
| The compound of Formula III or a salt thereof | 18-22 |
| The compound of Formula IV or a salt or solvate thereof | 50-65 |
| Microcrystalline cellulose | 7.5-11.5 |
| Crospovidone | 5.5-6.5 |
| Magnesium stearate | 1.3-1.7 |

In one embodiment, the dosage form comprises:

| Ingredient | % (w/w) |
|---|---|
| The compound of Formula I or a salt thereof | 0.64-0.75 |
| The compound of Formula II or a salt thereof | 3.3-3.9 |
| The compound of Formula III or a salt thereof | 18.3-21.6 |
| The compound of Formula IV or a salt or solvate thereof | 52-63 |
| Microcrystalline cellulose | 7.8-10.8 |
| Crospovidone | 5.5-6.3 |
| Magnesium stearate | 1.4-1.6 |

In one embodiment, the dosage form comprises:

| Ingredient | % (w/w) |
|---|---|
| The compound of Formula I or a salt thereof | 0.7 ± 0.05 |
| The compound of Formula II or a salt thereof | 3.8 ± 0.1 |
| The compound of Formula III or a salt thereof | 10 ± 1 |
| The compound of Formula IV or a salt or solvate thereof | 57 ± 2 |
| Silicon dioxide particles | 10 ± 1 |
| Microcrystalline cellulose | 10.3 ± 0.5 |
| Crospovidone | 6 ± 0.3 |
| Magnesium stearate | 1.5 ± 0.1 |

In one embodiment, the dosage form comprises:

| Ingredient | % (w/w) |
|---|---|
| The compound of Formula I or a salt thereof | 0.7 ± 0.05 |
| The compound of Formula II or a salt thereof | 3.6 ± 0.1 |
| The compound of Formula III or a salt thereof | 10 ± 1 |
| The compound of Formula IV or a salt or solvate thereof | 60 ± 3 |
| Silicon dioxide particles | 10 ± 1 |
| Microcrystalline cellulose | 8.5 ± 0.5 |
| Crospovidone | 6 ± 0.3 |
| Magnesium stearate | 1.5 ± 0.1 |

In one embodiment, the dosage form comprises:

| | |
|---|---|
| Ingredient | % (w/w) |

Intragranular

| | |
|---|---|
| The compound of Formula I or a salt thereof | 0.7 ± 0.05 |
| The compound of Formula II or a salt thereof | 3.8 ± 0.1 |
| The compound of Formula III or a salt thereof | 10 ± 1 |
| The compound of Formula IV or a salt or solvate thereof | 57 ± 2 |
| Silicon dioxide particles | 10 ± 1 |
| Microcrystalline cellulose | 10.3 ± 0.5 |
| Crospovidone | 6 ± 0.3 |
| Magnesium stearate | 0.75 ± 0.05 |

Extragranular

| | |
|---|---|
| Magnesium stearate | 0.75 ± 0.05 |

In one embodiment, the dosage form comprises:

| | |
|---|---|
| Ingredient | % (w/w) |

Intragranular

| | |
|---|---|
| The compound of Formula I or a salt thereof | 0.7 ± 0.05 |
| The compound of Formula II or a salt thereof | 3.6 ± 0.1 |
| The compound of Formula III or a salt thereof | 10 ± 1 |
| The compound of Formula IV or a salt or solvate thereof | 60 ± 3 |
| Silicon dioxide particles | 10 ± 1 |
| Microcrystalline cellulose | 8.5 ± 0.5 |
| Crospovidone | 6 ± 0.3 |
| Magnesium stearate | 0.75 ± 0.05 |

Extragranular

| | |
|---|---|
| Magnesium stearate | 0.75 ± 0.05 |

In one embodiment, the dosage form comprises:

| Ingredient | Mass (mg) |
|---|---|
| The compound of Formula I or a salt thereof | 10-30 |
| The compound of Formula II or a salt thereof | 25-75 |
| The compound of Formula III or a salt thereof | 150-350 |
| The compound of Formula IV or a salt or solvate thereof | 600-1000 |
| Microcrystalline cellulose | 100-175 |
| Crospovidone | 65-105 |
| Magnesium stearate | 10-30 |

In one embodiment, the dosage form comprises:

| Ingredient | Mass (mg) |
|---|---|
| The compound of Formula I or a salt thereof | 10-15 |
| The compound of Formula II or a salt thereof | 40-60 |
| The compound of Formula III or a salt thereof and silicon | 230-320 |
| dioxide particles | |
| The compound of Formula IV or a salt or solvate thereof | 700-900 |
| Microcrystalline cellulose | 110-165 |
| Crospovidone | 75-100 |
| Magnesium stearate | 15-25 |

In one embodiment, the dosage form comprises:

| Ingredient | Mass (mg) |
|---|---|
| The compound of Formula I or a salt thereof | 10-12 |
| The compound of Formula II or a salt thereof | 40-55 |
| The compound of Formula III or a salt thereof and silicon | 260-310 |
| dioxide particles | |
| The compound of Formula IV or a salt or solvate thereof | 800-900 |
| Microcrystalline cellulose | 115-155 |
| Crospovidone | 83-93 |
| Magnesium stearate | 18-24 |

In one embodiment, the dosage form comprises:

| Ingredient | Mass (mg) |
|---|---|
| The compound of Formula I or a salt thereof | 10-10.5 |
| The compound of Formula II or a salt thereof | 50-55 |
| The compound of Formula III or a salt thereof and silicon | 275-300 |
| dioxide particles | |
| The compound of Formula IV or a salt or solvate thereof | 800-875 |
| Microcrystalline cellulose | 116-150 |
| Crospovidone | 85-90 |
| Magnesium stearate | 20-22 |

In one embodiment, the dosage form comprises:

| Ingredient | Mass (mg) |
|---|---|
| The compound of Formula I or a salt thereof | 10 ± 0.5 |
| The compound of Formula II or a salt thereof | 50 ± 2.5 |
| The compound of Formula III or a salt thereof | 150 ± 7.5 |
| The compound of Formula IV or a salt or solvate thereof | 800 ± 40 |
| Silicon dioxide particles | 140 ± 7 |
| Microcrystalline cellulose | 144 ± 7 |
| Crospovidone | 84 ± 4.2 |
| Magnesium stearate | 11 ± 0.65 |

In one embodiment, the dosage form comprises:

| Ingredient | Mass (mg) |
|---|---|
| The compound of Formula I or a salt thereof | 10 ± 0.5 |
| The compound of Formula II or a salt thereof | 50 ± 2.5 |
| The compound of Formula III or a salt thereof | 150 ± 7.5 |
| The compound of Formula IV or a salt or solvate thereof | 800 ± 40 |
| Silicon dioxide particles | 140 ± 7 |
| Microcrystalline cellulose | 123 ± 6 |
| Crospovidone | 87 ± 4.4 |
| Magnesium stearate | 11.8 ± 0.6 |

In one embodiment, the dosage form comprises:

| | |
|---|---|
| Ingredient | Mass (mg) |

Intragranular

| | |
|---|---|
| The compound of Formula I or a salt thereof | 10 ± 0.5 |
| The compound of Formula II or a salt thereof | 50 ± 2.5 |
| The compound of Formula III or a salt thereof | 150 ± 7.5 |
| The compound of Formula IV or a salt or solvate thereof | 800 ± 40 |
| Silicon dioxide particles | 140 ± 7 |
| Microcrystalline cellulose | 144 ± 7 |
| Crospovidone | 84 ± 4.2 |
| Magnesium stearate | 10.5 ± 0.5 |

Extragranular

| | |
|---|---|
| Magnesium stearate | 10.5 ± 0.5 |

In one embodiment, the dosage form comprises:

| | |
|---|---|
| Ingredient | Mass (mg) |

Intragranular

| | |
|---|---|
| The compound of Formula I or a salt thereof | 10 ± 0.5 |
| The compound of Formula II or a salt thereof | 50 ± 2.5 |
| The compound of Formula III or a salt thereof | 150 ± 7.5 |
| The compound of Formula IV or a salt or solvate thereof | 800 ± 40 |
| Silicon dioxide particles | 140 ± 7 |
| Microcrystalline cellulose | 123 ± 6 |
| Crospovidone | 87 ± 4.4 |
| Magnesium stearate | 10.9 ± 0.5 |

Extragranular

| | |
|---|---|
| Magnesium stearate | 10.9 ± 0.5 |

In one embodiment, the solid dosage form comprises:

| Ingredient | % (w/w) |
|---|---|
| The compound of Formula I or a salt thereof | 0.8-3.3 |
| The compound of Formula II or a salt thereof | 2.0-6.0 |
| The compound of Formula III or a salt thereof | 2.0-20.0 |
| The compound of Formula IV or a salt or | 40-75 |
| solvate thereof | |
| Microcrystalline cellulose | 5-15 |
| Crospovidone | 4-8 |
| Magnesium stearate | 0.2-1.8 |

In one embodiment, the solid dosage form comprises:

| Ingredient | % (w/w) |
|---|---|
| The compound of Formula I or a salt thereof | 1.0-3.1 |
| The compound of Formula II or a salt thereof | 1.5-4.5 |
| The compound of Formula III or a salt thereof | 4.0-18.0 |
| The compound of Formula IV or a salt or | 50-70 |
| solvate thereof | |
| Microcrystalline cellulose | 5-15 |
| Crospovidone | 5-7 |
| Magnesium stearate | 0.4-1.5 |

In one embodiment, the solid dosage form comprises:

| Ingredient | % (w/w) |
|---|---|
| The compound of Formula I or a salt thereof | 1.2-2.9 |
| The compound of Formula II or a salt thereof | 1.8-4.2 |
| The compound of Formula III or a salt thereof | 6-15 |
| The compound of Formula IV or a salt or | 52-66 |
| solvate thereof | |
| Microcrystalline cellulose | 5-15 |
| Crospovidone | 5-7 |
| Magnesium stearate | 0.4-1.5 |

In one embodiment, the solid dosage form comprises:

| Ingredient | % (w/w) |
|---|---|
| The compound of Formula I or a salt thereof | 1.4-2.7 |
| The compound of Formula II or a salt thereof | 1.9-4.0 |
| The compound of Formula III or a salt thereof | 7-13 |
| The compound of Formula IV or a salt or | 54-64 |
| solvate thereof | |
| Microcrystalline cellulose | 5-15 |
| Crospovidone | 5-7 |
| Magnesium stearate | 0.4-1.5 |

In one embodiment, the solid dosage form comprises:

| Ingredient | % (w/w) |
|---|---|
| The compound of Formula I or a salt thereof | 1.9-2.3 |
| The compound of Formula II or a salt thereof | 2.0-3.0 |
| The compound of Formula III or a salt thereof | 8-12 |
| The compound of Formula IV or a salt or | 56-62 |
| solvate thereof | |
| Microcrystalline cellulose | 5-15 |
| Crospovidone | 5-7 |
| Magnesium stearate | 0.4-1.5 |

In one embodiment, the solid dosage form comprises:

| Ingredient | % (w/w) |
|---|---|
| The compound of Formula I or a salt thereof | 1.4-2.8 |
| The compound of Formula II or a salt thereof | 2.1-2.8 |
| The compound of Formula III or a salt thereof | 9-11 |
| The compound of Formula IV or a salt or | 57-61 |
| solvate thereof | |
| Microcrystalline cellulose | 5-15 |
| Crospovidone | 5-7 |
| Magnesium stearate | 0.4-1.5 |

In one embodiment, the solid dosage form comprises:

| Ingredient | % (w/w) |
|---|---|
| The compound of Formula I or a salt thereof | 1.7-2.4 |
| The compound of Formula II or a salt thereof | 2.0-2.5 |
| The compound of Formula III or a salt thereof | 10-11 |
| The compound of Formula IV or a salt or | 58-60 |
| solvate thereof | |
| Microcrystalline cellulose | 5-15 |
| Crospovidone | 5-7 |
| Magnesium stearate | 0.4-1.5 |

In one embodiment, the solid dosage form comprises:

| Ingredient | % (w/w) |
|---|---|
| The compound of Formula I or a salt thereof | 1.4-2.8 |
| The compound of Formula II or a salt thereof | 2.1-2.8 |
| The compound of Formula III or a salt thereof | 19-21 |
| adsorbed onto a solid carrier | |
| The compound of Formula IV or a salt or | 57-61 |
| solvate thereof | |
| Microcrystalline cellulose | 5-15 |
| Crospovidone | 5-7 |
| Magnesium stearate | 0.4-1.5 |

In one embodiment, the solid dosage form comprises:

| Ingredient | % (w/w) |
|---|---|
| The compound of Formula I or a salt thereof | 2.0 ± 0.5 |
| The compound of Formula II or a salt thereof | 2.0 ± 0.5 |
| The compound of Formula III or a salt thereof | 10.0 ± 0.5 |
| The compound of Formula IV or a salt or | 60 ± 0.4 |
| solvate thereof | |
| Microcrystalline cellulose | 10 ± 2 |
| Crospovidone | 5 ± 1 |
| Magnesium stearate | 0.5 ± 0.05 |

In one embodiment, the solid dosage form comprises:

| Ingredient | % (w/w) |
|---|---|
| The compound of Formula I or a salt thereof | 2.0 ± 0.5 |
| The compound of Formula II or a salt thereof | 2.0 ± 0.2 |
| The compound of Formula III or a salt thereof | 10.0 ± 0.2 |
| The compound of Formula IV or a salt or | 60.0 ± 0.2 |
| solvate thereof | |
| Microcrystalline cellulose | 10 ± 1 |
| Crospovidone | 6.0 ± 0.5 |
| Magnesium stearate | 0.5 ± 0.05 |

In one embodiment, the solid dosage form comprises:

| | |
|---|---|
| Ingredient | % (w/w) |

Intragranular

| | |
|---|---|
| The compound of Formula I or a salt thereof | 2.0 ± 0.5 |
| The compound of Formula II or a salt thereof | 2.0 ± 0.5 |
| The compound of Formula III or a salt thereof | 10.0 ± 0.5 |
| The compound of Formula IV or a salt or | 60 ± 0.4 |
| solvate thereof | |
| Microcrystalline cellulose | 10 ± 2 |
| Crospovidone | 5 ± 1 |
| Magnesium stearate | 0.5 ± 0.05 |

Extragranular

| | |
|---|---|
| Magnesium stearate | 0.5 ± 0.05 |

In one embodiment, the dosage solid form comprises:

| | |
|---|---|
| Ingredient | % (w/w) |

Intragranular

| | |
|---|---|
| The compound of Formula I or a salt thereof | 2.0 ± 0.5 |
| The compound of Formula II or a salt thereof | 2.0 ± 0.2 |
| The compound of Formula III or a salt thereof | 10.0 ± 0.2 |
| The compound of Formula IV or a salt or | 60.0 ± 0.2 |
| solvate thereof | |
| Microcrystalline cellulose | 10 ± 1 |
| Crospovidone | 6.0 ± 0.5 |
| Magnesium stearate | 0.5 ± 0.05 |

Extragranular

| | |
|---|---|
| Magnesium stearate | 0.5 ± 0.05 |

In one embodiment, the dosage solid form comprises:

| Ingredient | % (w/w) |
|---|---|
| The compound of Formula I or a salt thereof | 2.0 ± 0.5 |
| The compound of Formula II or a salt thereof | 2.0 ± 0.5 |
| The compound of Formula III or a salt thereof | 20.0 ± 0.5 |
| on a solid carrier | |
| The compound of Formula IV or a salt or | 60 ± 0.4 |
| solvate thereof | |
| Microcrystalline cellulose | 10 ± 2 |
| Crospovidone | 5 ± 1 |
| Magnesium stearate | 0.5 ± 0.05 |

In one embodiment, the dosage solid form comprises:

| Ingredient | Mass (mg) |
|---|---|
| The compound of Formula I or a salt thereof | 13-48 |
| The compound of Formula II or a salt thereof | 15-50 |
| The compound of Formula III or a salt thereof | 100-400 |
| The compound of Formula IV or a salt or | 350-1350 |
| solvate thereof | |
| Microcrystalline cellulose | 115-150 |
| Crospovidone | 35-140 |
| Magnesium stearate | 3-12 |

In one embodiment, the solid dosage form comprises:

| Ingredient | Mass (mg) |
|---|---|
| The compound of Formula I or a salt thereof | 15-45 |
| The compound of Formula II or a salt thereof | 16-47 |
| The compound of Formula III or a salt thereof | 110-300 |
| The compound of Formula IV or a salt or | 430-1300 |
| solvate thereof | |
| Microcrystalline cellulose | 70-190 |
| Crospovidone | 40-130 |
| Magnesium stearate | 4-11 |

In one embodiment, the solid dosage form comprises:

| Ingredient | Mass (mg) |
|---|---|
| The compound of Formula I or a salt thereof | 20-40 |
| The compound of Formula II or a salt thereof | 19-45 |
| The compound of Formula III or a salt thereof | 125-250 |
| The compound of Formula IV or a salt or | 520-1200 |
| solvate thereof | |
| Microcrystalline cellulose | 80-180 |
| Crospovidone | 50-120 |
| Magnesium stearate | 4-11 |

In one embodiment, the solid dosage form comprises:

| Ingredient | Mass (mg) |
|---|---|
| The compound of Formula I or a salt thereof | 25-35 |
| The compound of Formula II or a salt thereof | 22-40 |
| The compound of Formula III or a salt thereof | 130-200 |
| The compound of Formula IV or a salt or | 600-1100 |
| solvate thereof | |
| Microcrystalline cellulose | 90-170 |
| Crospovidone | 60-110 |
| Magnesium stearate | 5-9 |

In one embodiment, the solid dosage form comprises:

| Ingredient | Mass (mg) |
|---|---|
| The compound of Formula I or a salt thereof | 28-32 |
| The compound of Formula II or a salt thereof | 25-37 |
| The compound of Formula III or a salt thereof | 135-180 |
| The compound of Formula IV or a salt or | 690-1040 |
| solvate thereof | |
| Microcrystalline cellulose | 100-160 |
| Crospovidone | 70-100 |
| Magnesium stearate | 6-8 |

In one embodiment, the solid dosage form comprises:

| Ingredient | Mass (mg) |
|---|---|
| The compound of Formula I or a salt thereof | 29-31 |
| The compound of Formula II or a salt thereof | 28-35 |
| The compound of Formula III or a salt thereof | 140-170 |
| The compound of Formula IV or a salt or | 780-950 |
| solvate thereof | |
| Microcrystalline cellulose | 115-140 |
| Crospovidone | 75-95 |
| Magnesium stearate | 7-8 |

In one embodiment, the solid dosage form comprises:

| Ingredient | Mass (mg) |
|---|---|
| The compound of Formula I or a salt thereof | 29-30 |
| The compound of Formula II or a salt thereof | 28-35 |
| The compound of Formula III or a salt thereof | 145-160 |
| The compound of Formula IV or a salt or | 780-950 |
| solvate thereof | |
| Microcrystalline cellulose | 115-140 |
| Crospovidone | 75-95 |
| Magnesium stearate | 7-8 |

In one embodiment, the solid dosage form comprises:

| Ingredient | Mass (mg) |
|---|---|
| The compound of Formula I or a salt thereof | 28-32 |
| The compound of Formula II or a salt thereof | 25-37 |
| The compound of Formula III or a salt thereof | 230-345 |
| on a solid carrier | |
| The compound of Formula IV or a salt or | 690-1040 |
| solvate thereof | |
| Microcrystalline cellulose | 100-160 |
| Crospovidone | 70-100 |
| Magnesium stearate | 6-8 |

In one embodiment, the solid dosage form comprises:

| Ingredient | Mass (mg) |
|---|---|
| The compound of Formula I or a salt thereof | 30 ± 5 |
| The compound of Formula II or a salt thereof | 30 ± 5 |
| The compound of Formula III or a salt thereof | 150 ± 30 |
| The compound of Formula IV or a salt or | 850 ± 50 |
| solvate thereof | |
| Microcrystalline cellulose | 110 ± 40 |
| Crospovidone | 85 ± 5 |
| Magnesium stearate | 6 ± 2 |

In one embodiment, the solid dosage form comprises:

| Ingredient | Mass (mg) |
|---|---|
| The compound of Formula I or a salt thereof | 30 ± 3 |
| The compound of Formula II or a salt thereof | 30 ± 3 |
| The compound of Formula III or a salt thereof | 150 ± 15 |
| The compound of Formula IV or a salt or | 850 ± 30 |
| solvate thereof | |
| Microcrystalline cellulose | 110 ± 20 |
| Crospovidone | 85 ± 2 |
| Magnesium stearate | 6 ± 2 |

In one embodiment, the solid dosage form comprises:

| Ingredient | Mass (mg) |
|---|---|
| The compound of Formula I or a salt thereof | 30 ± 2 |
| The compound of Formula II or a salt thereof | 31 ± 2 |
| The compound of Formula III or a salt thereof | 150 ± 10 |
| The compound of Formula IV or a salt or | 850 ± 20 |
| solvate thereof | |
| Microcrystalline cellulose | 110 ± 20 |
| Crospovidone | 87 ± 1 |
| Magnesium stearate | 7 ± 1 |

In one embodiment, the solid dosage form comprises:

| | |
|---|---|
| Ingredient | Mass (mg) |

Intragranular

| | |
|---|---|
| The compound of Formula I or a salt thereof | 30 ± 2 |
| The compound of Formula II or a salt thereof | 30 ± 3 |
| The compound of Formula III or a salt thereof | 150 ± 15 |
| The compound of Formula IV or a salt or | 850 ± 30 |
| solvate thereof | |
| Microcrystalline cellulose | 110 ± 20 |
| Crospovidone | 85 ± 2 |
| Magnesium stearate | 6 ± 2 |

Extragranular

| | |
|---|---|
| Magnesium stearate | 6 ± 2 |

In one embodiment, the solid dosage form comprises:

| | |
|---|---|
| Ingredient | Mass (mg) |

Intragranular

| | |
|---|---|
| The compound of Formula I or a salt thereof | 30 ± 1 |
| The compound of Formula II or a salt thereof | 31 ± 1 |
| The compound of Formula III or a salt thereof | 150 ± 10 |
| The compound of Formula IV or a salt or | 850 ± 20 |
| solvate thereof | |
| Microcrystalline cellulose | 110 ± 20 |
| Crospovidone | 87 ± 1 |
| Magnesium stearate | 7 ± 1 |

Extragranular

| | |
|---|---|
| Magnesium stearate | 7 ± 1 |

In one embodiment, the solid dosage form comprises:

| | |
|---|---|
| Ingredient | Mass (mg) |

Intragranular

| | |
|---|---|
| The compound of Formula I or a salt thereof | 30 ± 2 |
| The compound of Formula II or a salt thereof | 30 ± 3 |
| The compound of Formula III or a salt thereof | 280 ± 15 |
| on a solid carrier | |
| The compound of Formula IV or a salt or | 850 ± 30 |
| solvate thereof | |
| Microcrystalline cellulose | 110 ± 20 |
| Crospovidone | 85 ± 2 |
| Magnesium stearate | 6 ± 2 |

Extragranular

| | |
|---|---|
| Magnesium stearate | 6 ± 2 |

In one embodiment, the solid dosage form comprises:

| Ingredient | % (w/w) |
|---|---|
| The compound of Formula I or a salt thereof | 2.5-5.8 |
| The compound of Formula II or a salt thereof | 1.8-4.2 |
| The compound of Formula III or a salt thereof | 4-18 |
| The compound of Formula IV or a salt or | 52-66 |
| solvate thereof | |
| Microcrystalline cellulose | 5.3-8.6 |
| Crospovidone | 5-7 |
| Magnesium stearate | 0.1-1.6 |

In one embodiment, the solid dosage form comprises:

| Ingredient | % (w/w) |
|---|---|
| The compound of Formula I or a salt thereof | 2.9-5.4 |
| The compound of Formula II or a salt thereof | 1.9-4.0 |
| The compound of Formula III or a salt thereof | 6-15 |
| The compound of Formula IV or a salt or | 54-64 |
| solvate thereof | |
| Microcrystalline cellulose | 5.7-8.2 |
| Crospovidone | 5-7 |
| Magnesium stearate | 0.1-1.6 |

In one embodiment the solid dosage form comprises:

| Ingredient | % (w/w) |
|---|---|
| The compound of Formula I or a salt thereof | 3.3-5.0 |
| The compound of Formula II or a salt thereof | 2.0-3.0 |
| The compound of Formula III or a salt thereof | 7-13 |
| The compound of Formula IV or a salt or | 56-62 |
| solvate thereof | |
| Microcrystalline cellulose | 6.1-7.8 |
| Crospovidone | 5-7 |
| Magnesium stearate | 0.2-1.5 |

In one embodiment, the solid dosage form comprises:

| Ingredient | % (w/w) |
|---|---|
| The compound of Formula I or a salt thereof | 3.7-4.3 |
| The compound of Formula II or a salt thereof | 2.1-2.8 |
| The compound of Formula III or a salt thereof | 8-12 |
| The compound of Formula IV or a salt or | 57-61 |
| solvate thereof | |
| Microcrystalline cellulose | 6.5-7.4 |
| Crospovidone | 5-7 |
| Magnesium stearate | 0.3-1.4 |

In one embodiment, the solid dosage form comprises:

| Ingredient | % (w/w) |
|---|---|
| The compound of Formula I or a salt thereof | 3.8-4.3 |
| The compound of Formula II or a salt thereof | 2.0-2.5 |
| The compound of Formula III or a salt thereof | 9-11 |
| The compound of Formula IV or a salt or | 58-60 |
| solvate thereof | |
| Microcrystalline cellulose | 6.8-7.2 |
| Crospovidone | 5-7 |
| Magnesium stearate | 0.4-1.2 |

In one embodiment, the solid dosage form comprises:

| Ingredient | % (w/w) |
|---|---|
| The compound of Formula I or a salt thereof | 3.9-4.1 |
| The compound of Formula II or a salt thereof | 2.1-2.2 |
| The compound of Formula III or a salt thereof | 10-11 |
| The compound of Formula IV or a salt or | 59-60 |
| solvate thereof | |
| Microcrystalline cellulose | 6.9-7.1 |
| Crospovidone | 5.5-6.5 |
| Magnesium stearate | 0.4-1.0 |

In one embodiment, the solid dosage form comprises:

| Ingredient | % (w/w) |
|---|---|
| The compound of Formula I or a salt thereof | 3.3-5.0 |
| The compound of Formula II or a salt thereof | 2.0-3.0 |
| The compound of Formula III or a salt thereof | 19.0-20.5 |
| adsorbed on a solid carrier | |
| The compound of Formula IV or a salt or | 56-62 |
| solvate thereof | |
| Microcrystalline cellulose | 6.1-7.8 |
| Crospovidone | 5-7 |
| Magnesium stearate | 0.2-1.5 |

In one embodiment, the solid dosage form comprises:

| Ingredient | % (w/w) |
|---|---|
| The compound of Formula I or a salt thereof | 4.0 ± 0.5 |
| The compound of Formula II or a salt thereof | 2.0 ± 0.5 |
| The compound of Formula III or a salt thereof | 10.0 ± 0.5 |
| The compound of Formula IV or a salt or | 60 ± 0.4 |
| solvate thereof | |
| Microcrystalline cellulose | 7.0 ± 1 |
| Crospovidone | 5 ± 1 |
| Magnesium stearate | 0.5 ± 0.05 |

In one embodiment, the solid dosage form comprises:

| Ingredient | % (w/w) |
|---|---|
| The compound of Formula I or a salt thereof | 4.0 ± 0.3 |
| The compound of Formula II or a salt thereof | 2.0 ± 0.2 |
| The compound of Formula III or a salt thereof | 10.0 ± 0.2 |
| The compound of Formula IV or a salt or | 60.0 ± 0.2 |
| solvate thereof | |
| Microcrystalline cellulose | 7.0 ± 0.5 |
| Crospovidone | 6.0 ± 0.5 |
| Magnesium stearate | 0.5 ± 0.05 |

In one embodiment, the solid dosage form comprises:

| | |
|---|---|
| Ingredient | % (w/w) |

Intragranular

| | |
|---|---|
| The compound of Formula I or a salt thereof | 4.0 ± 0.5 |
| The compound of Formula II or a salt thereof | 2.0 ± 0.5 |
| The compound of Formula III or a salt thereof | 10.0 ± 0.5 |
| The compound of Formula IV or a salt or | 60 ± 0.4 |
| solvate thereof | |
| Microcrystalline cellulose | 7.0 ± 1 |
| Crospovidone | 5 ± 1 |
| Magnesium stearate | 0.5 ± 0.05 |

Extragranular

| | |
|---|---|
| Magnesium stearate | 0.5 ± 0.05 |

In one embodiment, the solid dosage form comprises:

| | |
|---|---|
| Ingredient | % (w/w) |

Intragranular

| | |
|---|---|
| The compound of Formula I or a salt thereof | 4.0 ± 0.3 |
| The compound of Formula II or a salt thereof | 2.0 ± 0.2 |
| The compound of Formula III or a salt thereof | 10.0 ± 0.2 |
| The compound of Formula IV or a salt or | 60.0 ± 0.2 |
| solvate thereof | |
| Microcrystalline cellulose | 7.0 ± 0.5 |
| Crospovidone | 6.0 ± 0.5 |
| Magnesium stearate | 0.5 ± 0.05 |

Extragranular

| | |
|---|---|
| Magnesium stearate | 0.5 ± 0.05 |

In one embodiment, the solid dosage form comprises:

| | |
|---|---|
| Ingredient | % (w/w) |

Intragranular

| | |
|---|---|
| The compound of Formula I or a salt thereof | 4.0 ± 0.3 |
| The compound of Formula II or a salt thereof | 2.0 ± 0.2 |
| The compound of Formula III or a salt thereof | 20.0 ± 0.2 |
| adsorbed onto a solid carrier | |
| The compound of Formula IV or a salt or | 60.0 ± 0.2 |
| solvate thereof | |
| Microcrystalline cellulose | 7.0 ± 0.5 |
| Crospovidone | 6.0 ± 0.5 |
| Magnesium stearate | 0.5 ± 0.05 |

Extragranular

| | |
|---|---|
| Magnesium stearate | 0.5 ± 0.05 |

In one embodiment, the solid dosage form comprises:

| Ingredient | Mass (mg) |
|---|---|
| The compound of Formula I or a salt thereof | 36-84 |
| The compound of Formula II or a salt thereof | 20-44 |
| The compound of Formula III or a salt thereof | 125-300 |
| The compound of Formula IV or a salt or | 522-1218 |
| solvate thereof | |
| Microcrystalline cellulose | 58-145 |
| Crospovidone | 50-120 |
| Magnesium stearate | 4-11 |

In one embodiment, the solid dosage form comprises:

| Ingredient | Mass (mg) |
|---|---|
| The compound of Formula I or a salt thereof | 42-78 |
| The compound of Formula II or a salt thereof | 22-40 |
| The compound of Formula III or a salt thereof | 130-200 |
| The compound of Formula IV or a salt or | 600-1100 |
| solvate thereof | |
| Microcrystalline cellulose | 73-131 |
| Crospovidone | 60-110 |
| Magnesium stearate | 5-9 |

In one embodiment, the solid dosage form comprises:

| Ingredient | Mass (mg) |
|---|---|
| The compound of Formula I or a salt thereof | 48-72 |
| The compound of Formula II or a salt thereof | 25-37 |
| The compound of Formula III or a salt thereof | 135-180 |
| The compound of Formula IV or a salt or | 690-1040 |
| solvate thereof | |
| Microcrystalline cellulose | 81-123 |
| Crospovidone | 70-100 |
| Magnesium stearate | 6-8 |

In one embodiment, the solid dosage form comprises:

| Ingredient | Mass (mg) |
|---|---|
| The compound of Formula I or a salt thereof | 54-66 |
| The compound of Formula II or a salt thereof | 28-35 |
| The compound of Formula III or a salt thereof | 140-170 |
| The compound of Formula IV or a salt or | 780-950 |
| solvate thereof | |
| Microcrystalline cellulose | 91-112 |
| Crospovidone | 75-95 |
| Magnesium stearate | 7-8 |

In one embodiment, the solid dosage form comprises:

| Ingredient | Mass (mg) |
|---|---|
| The compound of Formula I or a salt thereof | 59-62 |
| The compound of Formula II or a salt thereof | 28-35 |
| The compound of Formula III or a salt thereof | 145-160 |
| The compound of Formula IV or a salt or | 780-950 |
| solvate thereof | |
| Microcrystalline cellulose | 94-109 |
| Crospovidone | 75-95 |
| Magnesium stearate | 7-8 |

In one embodiment, the solid dosage form comprises:

| Ingredient | Mass (mg) |
|---|---|
| The compound of Formula I or a salt thereof | 58-61 |
| The compound of Formula II or a salt thereof | 31-33 |
| The compound of Formula III or a salt thereof | 145-155 |
| The compound of Formula IV or a salt or | 845-889 |
| solvate thereof | |
| Microcrystalline cellulose | 99-104 |
| Crospovidone | 85-89 |
| Magnesium stearate | 7-8 |

In one embodiment, the solid dosage form comprises:

| Ingredient | Mass (mg) |
|---|---|
| The compound of Formula I or a salt thereof | 54-66 |
| The compound of Formula II or a salt thereof | 28-35 |
| The compound of Formula III or a salt thereof | 260-310 |
| adsorbed onto a solid carrier | |
| The compound of Formula IV or a salt or | 780-950 |
| solvate thereof | |
| Microcrystalline cellulose | 91-112 |
| Crospovidone | 75-95 |
| Magnesium stearate | 7-8 |

In one embodiment, the solid dosage form comprises:

| Ingredient | Mass (mg) |
|---|---|
| The compound of Formula I or a salt thereof | 60 ± 10 |
| The compound of Formula II or a salt thereof | 30 ± 5 |
| The compound of Formula III or a salt thereof | 150 ± 50 |
| The compound of Formula IV or a salt or | 860 ± 150 |
| solvate thereof | |
| Microcrystalline cellulose | 100 ± 20 |
| Crospovidone | 90 ± 16 |
| Magnesium stearate | 7 ± 2 |

In one embodiment, the solid dosage form comprises:

| Ingredient | Mass (mg) |
|---|---|
| The compound of Formula I or a salt thereof | 60 ± 5 |
| The compound of Formula II or a salt thereof | 31 ± 3 |
| The compound of Formula III or a salt thereof | 150 ± 30 |
| The compound of Formula IV or a salt or | 860 ± 80 |
| solvate thereof | |
| Microcrystalline cellulose | 100 ± 10 |
| Crospovidone | 90 ± 10 |
| Magnesium stearate | 7 ± 2 |

In one embodiment, the solid dosage form comprises:

| Ingredient | Mass (mg) |
|---|---|
| The compound of Formula I or a salt thereof | 60 ± 2 |
| The compound of Formula II or a salt thereof | 31 ± 1 |
| The compound of Formula III or a salt thereof | 150 ± 10 |
| The compound of Formula IV or a salt or | 870 ± 50 |
| solvate thereof | |
| Microcrystalline cellulose | 100 ± 5 |
| Crospovidone | 90 ± 5 |
| Magnesium stearate | 7 ± 2 |

In one embodiment, the solid dosage form comprises:

| | |
|---|---|
| Ingredient | Mass (mg) |

Intragranular

| | |
|---|---|
| The compound of Formula I or a salt thereof | 60 ± 5 |
| The compound of Formula II or a salt thereof | 31 ± 3 |
| The compound of Formula III or a salt thereof | 150 ± 30 |
| The compound of Formula IV or a salt or | 860 ± 80 |
| solvate thereof | |
| Microcrystalline cellulose | 100 ± 10 |
| Crospovidone | 90 ± 10 |
| Magnesium stearate | 7 ± 2 |

Extragranular

| | |
|---|---|
| Magnesium stearate | 7 ± 2 |

In one embodiment, the solid dosage form comprises:

| | |
|---|---|
| Ingredient | Mass (mg) |

Intragranular

| | |
|---|---|
| The compound of Formula I or a salt thereof | 60 ± 2 |
| The compound of Formula II or a salt thereof | 31 ± 1 |
| The compound of Formula III or a salt thereof | 150 ± 10 |
| The compound of Formula IV or a salt or | 870 ± 50 |
| solvate thereof | |
| Microcrystalline cellulose | 100 ± 5 |
| Crospovidone | 90 ± 5 |
| Magnesium stearate | 7 ± 2 |

Extragranular

| | |
|---|---|
| Magnesium stearate | 7 ± 2 |

In one embodiment, the solid dosage form comprises:

| | |
|---|---|
| Ingredient | Mass (mg) |

Intragranular

| | |
|---|---|
| The compound of Formula I or a salt thereof | 60 ± 5 |
| The compound of Formula II or a salt thereof | 31 ± 3 |
| The compound of Formula III or a salt thereof | 280 ± 30 |
| adsorbed onto a solid carrier | |
| The compound of Formula IV or a salt or | 860 ± 80 |
| solvate thereof | |
| Microcrystalline cellulose | 100 ± 10 |
| Crospovidone | 90 ± 10 |
| Magnesium stearate | 7 ± 2 |

Extragranular

| | |
|---|---|
| Magnesium stearate | 7 ± 2 |

In one embodiment, the tablet further comprises a film coating. In one embodiment, the tablet comprises about 25 mg to about 60 mg of a film coating. In one embodiment, the tablet further comprises about 35 mg to about 55 mg of a film coating. In one embodiment, the tablet further comprises about 40 mg to about 50 mg of a film coating. In one embodiment, the tablet further comprises about 40 mg to about 45 mg of a film coating. In one embodiment, the tablet further comprises about 42 mg of a film coating. In one embodiment the film coating comprises polyvinyl alcohol, polyethylene glycol, talc, and titanium dioxide, and a coloring (e.g., one or more of black iron oxide, yellow iron oxide, red iron oxide). polyvinylalcohol, polyethylene glycol, titanium dioxide, talc, yellow iron oxide, and red iron oxide. In one embodiment the film coating consists of 42±5 mg of Opadry II Brown 85F165072 or 85F165010. In one embodiment the film coating consists of 44±5 mg of Opadry II Brown 85F165072 or 85F165010.

In one embodiment, the tablet further comprises about 1% to about 5% w/w of a film coating. In one embodiment, the tablet further comprises about 2% to about 4% w/w of a film coating. In one embodiment, the tablet further comprises about 2.5% to about 3.5% w/w of a film coating. In one embodiment, the tablet further comprises about 3% of a film coating. In one embodiment the film coating includes polyvinyl alcohol, polyethylene glycol, talc, titanium dioxide, iron oxide red, and black iron oxide. In one embodiment the film coating includes 36%-40% polyvinyl alcohol, 18%-22% polyethylene glycol, 13%-16% talc, 20%-24% titanium dioxide, 2%-3% iron oxide red, and 0.5%-0.7% black iron oxide.

### Pharmacokinetics

In the oral solid dosage form disclosed herein, four therapeutic compounds (Formulas I, II, III, and IV) have been manufactured into one fixed dose combination formulation tablet. Studies were performed to demonstrate bio-comparability, i.e. equivalent systemic exposure (AUC_{inf}, Cₘₐₓ) for each of the active ingredients compared to standard comparators. In particular, bio-compatibility was tested for each active component by comparing each active component (Formula I, Formula II, Formula III, and Formula IV) in the fixed dose combination formulation to a combination, i.e., co-administration, of single agent Formula I and single agent Formula II and co-formulated darunavir and cobicistat (Prezcobix®).

As used herein, C₀ is the observed plasma/serum concentration at time zero.

As used herein, Cₗₐₛₜ is the observed quantifiable plasma/serum concentration of the drug.

As used herein, Cₘₐₓ is the maximum observed plasma/serum concentration of drug.

As used herein, AUC_{inf} is the area under the plasma/serum concentration versus time curve extrapolated to infinite time, calculated as AUC₀₋ₗₐₛₜ +(Cₗₐₛₜ/λ_{z}).

As used herein, AUCₗₐₛₜ, is the area under the plasma/serum concentration versus time curve from time zero to the last quantifiable concentration.

As used herein, the term “fixed dose combination formulation tablet” used herein refers to a tablet containing fixed amounts of Formula I, Formula II, Formula III, and Formula IV.

As used herein, the term “co-administered” means that two or more agents (e.g. tablets) were given at the same time to a subject (e.g. human, dog, etc.).

Studies were carried out to determine the similarity and differences between plasma/serum concentration of Formula I, Formula II, Formula III, and Formula IV as contained within the fixed dose combination formulation tablet compared to co-administered single agent Formula I, single agent Formula II, and co-formulated darunavir and cobicistat (Prezcobix®). Formulation F11 was used in the PK studies (Example 7). The oral administration of fixed dose combination formulation tablet compared to co-administered single agent Formula I, single agent Formula II, and co-formulated darunavir and cobicistat in dogs (amounts of each disclosed in Example 6).

Bioanalytical Method

### Formula I Processing (20 uL)

To a 20 μL aliquot of each plasma sample with exception of the matrix blanks, 120 μL of a solution containing 50 nM SIT in 100:0.1 acetonitrile:formic acid (ACN:FA) was added. The matrix blank samples received 120 μL of 100:0.1 acetonitrile:formic acid only. The precipitated proteins were removed by centrifugation and 100 μL of supernatant was transferred into a clean 96 deep-well plate containing 10 μL of DMSO in each well. The solvent was evaporated under a flow of nitrogen at 45° C. to the level of the DMSO. Plates were reconstituted with 100 μL of 90:10:0.1 water:acetonitrile:formic acid. An aliquot 5-10 μL was injected into an Applied Biosystems API-5500 LC/MS/MS system.

### Column

Waters HSS T3 column (30×2.1 mm, 2.5 μm)

### Mobile Phases

Mobile phase A: 100:0.1 Water:Formic Acid
Mobile phase B: 100:0.1 Acetonitrile:Formic Acid

### HPLC Pumps

An Agilent 1200 series high pressure binary pump (SL) (G1312 B) was used for elution and separation as part of a Thermo Scientific (Cohesive) LX-2 multiplexed system.

### Autosampler

CTC PAL autosampler as part of a Thermo Scientific (Cohesive) LX-2 multiplexed system was used.

### HPLC Elution Program

| | | | Mobile Phase | Mobile Phase |
|---|---|---|---|---|
| Time (sec) | | Flow Rate (mL/min) | A (%) | B (%) |
| 15 | (Step) | 0.800 | 100 | 0 |
| 60 | (Ramp) | 0.800 | 60 | 40 |
| 5 | (Ramp) | 0.800 | 5 | 95 |
| 30 | (Step) | 0.800 | 5 | 95 |
| 40 | (Step) | 0.800 | 100 | 0 |

### Mass Spectrometry

### Mass Spectrometer

API-5500 triple quadrupole mass spectrometer from Applied Biosystems, Foster City, Calif.

### Operation Mode

Multiple reaction monitoring (MRM).

### Mass Spectrometry Parameters

| Ion | Curtain | Ion spray | Ion source | Ion source | Temperature |
|---|---|---|---|---|---|
| source | gas | voltage (V) | gas 1 | gas 2 | (° C.) |
| ESI+ | 20 | 5500 | 50 | 50 | 550 |

### Formula II, III, and IV Processing (20 uL)

To a 20 μL aliquot of each plasma sample with exception of the matrix blanks, 120 μL of a solution containing 50 nM of an internal standard for Formula II, 50 nM of an internal standard for Formula III, and 100 ng/mL of internal standard for Formula IV in acetonitrile (ACN) was added. The matrix blank samples received 120 μL of acetonitrile only. The precipitated proteins were removed by centrifugation and 100 μL of supernatant was transferred into a clean 96 deep-well plate. A 100 μL aliquot of water was added to each sample. An aliquot of 5-12 μL was injected into an Applied Biosystems API-5500 LC/MS/MS system.

### Column

Waters HSS T3 column (30×2.1 mm, 2.5 μm)

### Mobile Phases

Mobile phase A: 100:0.1 Water:Formic Acid
Mobile phase B: 100:0.1 Acetonitrile:Formic Acid

### HPLC Pumps

An Agilent 1200 series high pressure binary pump (SL) (G1312 B) was used for elution and separation as part of a Thermo Scientific (Cohesive) LX-2 multiplexed system.

### Autosampler

CTC PAL autosampler as part of a Thermo Scientific (Cohesive) LX-2 multiplexed system was used.

### HPLC Elution Program

| | Flow Rate | | |
|---|---|---|---|
| Time (sec) | (mL/min) | Mobile Phase A (%) | Mobile Phase B (%) |
| 15 (Step) | 0.800 | 70 | 30 |
| 60 (Ramp) | 0.800 | 30 | 70 |
| 5 (Ramp) | 0.800 | 5 | 95 |
| 30 (Step) | 0.800 | 5 | 95 |
| 40 (Step) | 0.800 | 70 | 30 |

### Mass Spectrometry

### Mass Spectrometer

API-5500 triple quadrupole mass spectrometer from Applied Biosystems, Foster City, Calif.

### Operation Mode

Multiple reaction monitoring (MRM).

### Mass Spectrometry Parameters

| Ion | Curtain | Ion spray | Ion source | Ion source | Temperature |
|---|---|---|---|---|---|
| source | gas | voltage (V) | gas 1 | gas 2 | (° C.) |
| ESI+ | 20 | 5500 | 50 | 50 | 550 |

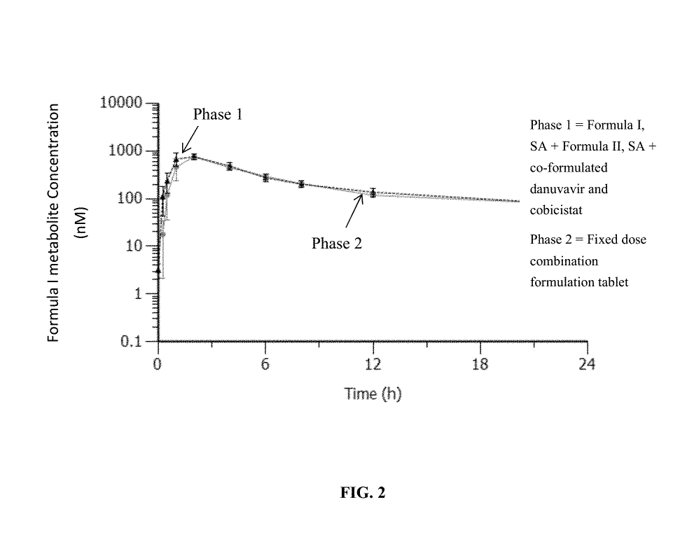

FIG. 2 is a graphical comparison of a quarter (25%) dose of Formulation F6 (Example 4) with co-administration of comparable amounts of single agent Formula I (Tablet Formulation F8) and single agent Formula II (Tablet Formulation F10) with co-formulated darunavir and cobicistat that was co-administered. In FIG. 2, Formulation F4 was prepared according to the amounts of Example 3 and manufactured according to the process of Example 11, and a 25% dose amount was used (Example 9). Single agent tablet formulations containing a compound of Formula I (Formulation F7 or F8) and a compound of Formula II (F9 or F10), respectively, were prepared according to Examples 5 and 6. The amounts of F7, F8, F9, or F10 were adjusted so that the same amounts of Formula I and Formula II were co-administered compared to the amounts of Example 9. Formulation F11 (quarter dose of Formulation F6) was administered to fasted dogs and their blood levels monitored. Formulation F12 was co-administered to fasted dogs and their blood levels monitored. Data points taken from time 0 hrs to approximately 24 hrs later show consistency of the mean AUCₗₐₛₜ, AUC_{inf}, and Cₘₐₓ values within standard deviations. Table 1 discloses PK values for the fixed dose combination formulation tablet and for the co-administered single agents. As FIG. 2 shows, the fitted curves for F11 and F12 administration were comparable.

TABLE 1
Comparison of PK (pharmacokinetic) values of the compound of
Formula I of F11 with Formula I of F12 in fasted dogs (n = 6)

| | | | | Single agent |
|---|---|---|---|---|
| | | | Fixed dose | Formula I, II, and |
| | | | combination | co-formulated |
| | Analyte | PK | formulation | darunavir and |
| Dose | analyzed | parameter | tablet (F11) | cobicistat (F12) |
| 15 mg | Formula I | Mean AUCₗₐₛₜ | 5390 (946) | 5000 (624) |
| | metabolite | (nM*hr) (SD) | | |
| | | Mean Cₘₐₓ | 771 (104) | 753 (84) |
| | | (nM) (SD) | | |

* Numbers in parentheses are standard deviations

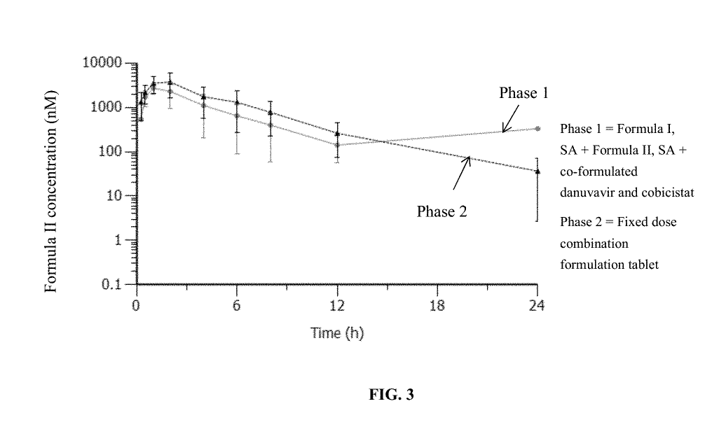

A graphical comparison of mean concentration over time of Formula II as compared to single agents of Formula I and Formula II with co-formulated darunavir and cobicistat is shown in FIG. 3. The PK values were obtained for a comparable amount of Formula II in F12 and the results are shown in Table 2. Overall, the PK values for Formula II within the fixed dose combination formulation tablet were comparable with the PK values for the single agents of Formula I and Formula II with co-formulated darunavir and cobicistat.

TABLE 2
Comparison of PK values of the compound of Formula II contained
within F11 with Formula II of F12 in fasted dogs (n = 6)

| | | | | Single agent |
|---|---|---|---|---|
| | | | Fixed dose | Formula I, II, and |
| | | | combination | co-formulated |
| | Analyte | PK | formulation | darunavir and |
| Dose | analyzed | parameter | tablet (F11) | cobicistat (F12) |
| 7.5 mg | Formula II | Mean AUCₗₐₛₜ | 20100 (11100) | 14100 (7470) |
| | | (nM*hr) (SD) | | |
| | | Mean Cₘₐₓ | 4440 (1570) | 2820 (988) |
| | | (nM) (SD) | | |

* Numbers in parentheses are standard deviations

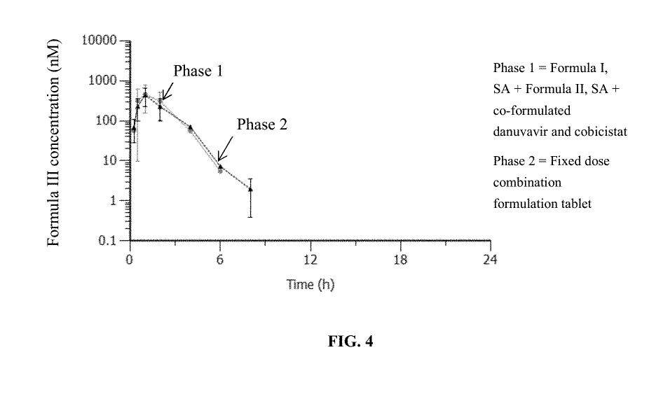

A graphical comparison of mean concentration over time of Formula III within a fixed dose combination formulation tablet was compared to co-administration of single agents of Formula I, single agent Formula II, and co-formulated darunavir and cobicistat is shown in FIG. 4. The comparison of PK values for Formula III within F11 and F12 are disclosed in Table 3. Overall, the PK values obtained for Formula III within the fixed dose combination formulation tablet were comparable with the PK values obtained for the single agents of Formula I and Formula II with co-formulated darunavir and cobicistat.

TABLE 3
Comparison of PK values of the compound of Formula
III of F11 with that of F12 in fasted dogs (n = 6)

| | | | | Single agent |
|---|---|---|---|---|
| | | | Fixed dose | Formula I, II, and |
| | | | combination | co-formulated |
| | Analyte | PK | formulation | darunavir and |
| Dose | analyzed | parameter | tablet (F11) | cobicistat (F12) |
| 37.5 mg | Formula III | Mean AUCₗₐₛₜ | 929 (530) | 1060 (722) |
| | | (nM*hr) (SD) | | |
| | | Mean Cₘₐₓ | 470 (173) | 524 (260) |
| | | (nM) (SD) | | |

* Numbers in parentheses are standard deviations

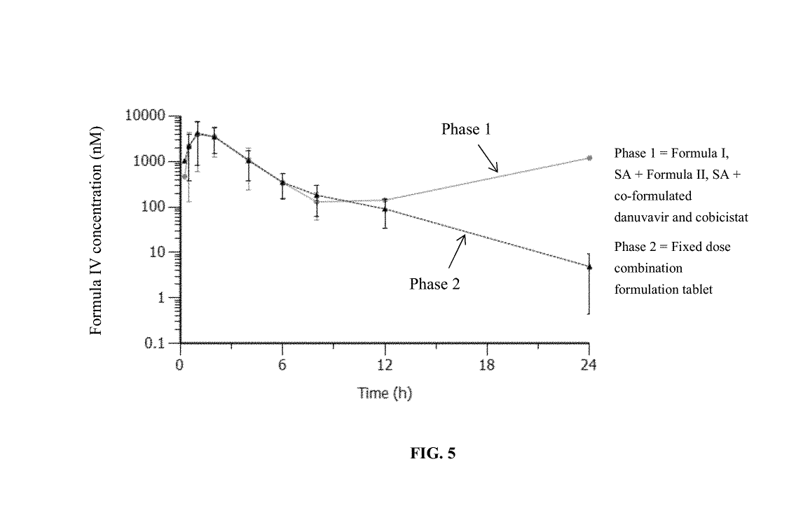

A graphical comparison of mean concentration over time of Formula IV as compared to single agents of Formula I and Formula II with co-formulated darunavir and cobicistat is shown in FIG. 5. The comparable mixture of single agent Formula I and Formula II with co-formulated darunavir and cobicistat (F12) were co-administered to the subjects. Table 4 discloses PK values for Formula IV as contained within F11 compared to that Formula IV as contained within F12. Similar to PK results for Formula I, II, and III, the mean concentration of Formula IV, administered as a fixed dose combination formulation tablet (F11) and as a mixture of single agents of Formula I and Formula II with co-formulated darunavir and cobicistat (F12) were not statistically different.

TABLE 4
Comparison of PK values of the compound of Formula
IV of F11 and F12 in fasted dogs (n = 6)

| | | | | Single agent |
|---|---|---|---|---|
| | | | Fixed dose | Formula I, II, and |
| | | | combination | co-formulated |
| | Analyte | PK | formulation | darunavir and |
| Dose | analyzed | parameter | tablet (F11) | cobicistat (F12) |
| 200 mg | Formula | Mean AUCₗₐₛₜ | 13500 (8030) | 20700 (26300) |
| | IV | (nM*hr) (SD) | | |
| | | Mean Cₘₐₓ | 4800 (2850) | 4350 (2990) |
| | | (nM) (SD) | | |

* Numbers in parentheses are standard deviations

Cₘₐₓ, Cₗₐₛₜ, AUC_{inf}, and AUCₗₐₛₜ are standard pharmacokinetic parameters that can be estimated manually from the measured amounts of the active ingredient in the blood as a function of time. PK values were obtained via the following bioanalytical methods described in Example 10.

### Stability Studies

The chemical stability of the active components in a pharmaceutical formulation is of concern to minimize the generation of impurities and ensure sufficient shelf-life. To follow the degradation of each of the active ingredients (Formula I, Formula II, Formula III, and Formula IV), stability studies were performed on the solid oral dosage form. Of the four active ingredients of the solid oral dosage form, Formula I and Formula IV have relatively low pKa values (3.7 and 2.0 respectively), indicative of a higher potential for hydrolysis.

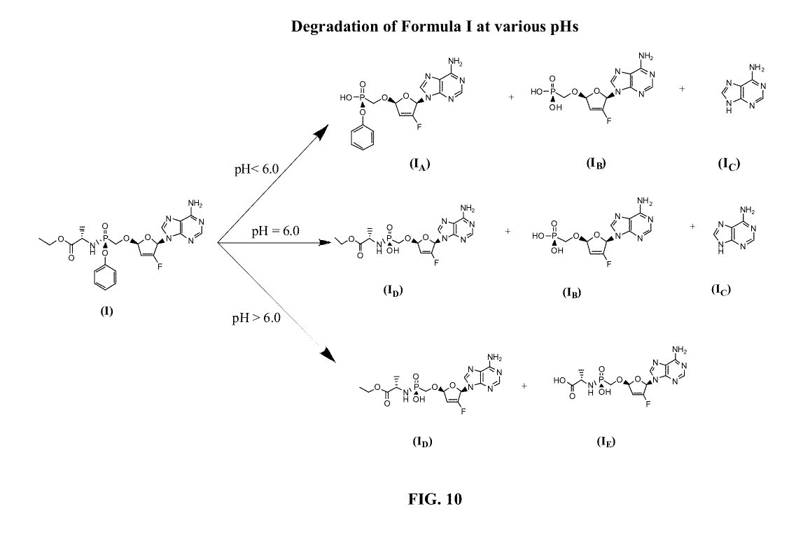

FIG. 10 shows degradation pathway of Formula I as a function of pH. In addition to the degradation products shown in FIG. 10, Formula I has other minor impurities and/or degradants shown below:

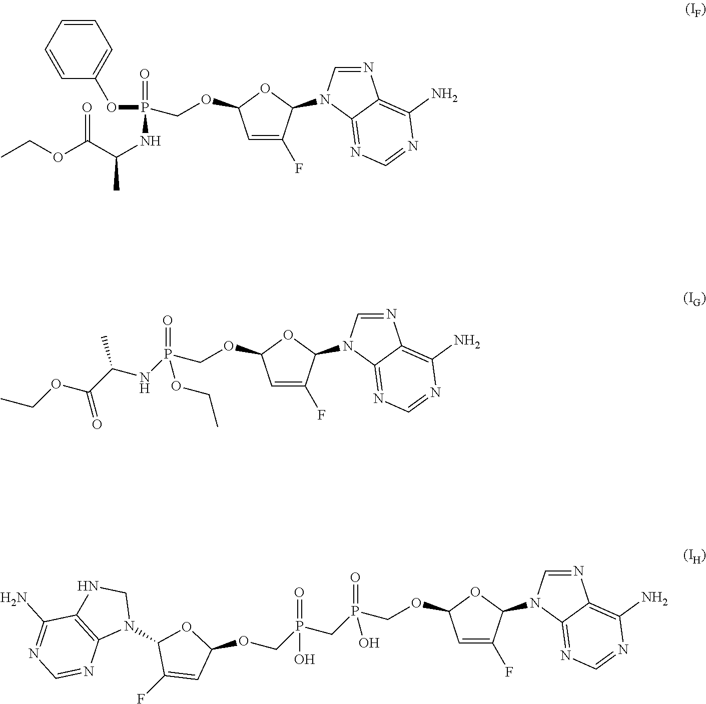

In general, the purity of the products and possible impurities were measured using ultra high performance liquid chromatography (UPLC). The purity measurement by % area normalization of the chromatographic peaks were performed at initial time zero and then again at a subsequent time. The chromatographic profile from the initial LC run was then compared to the peaks obtained at the later time. The UPLC conditions were as follows:

Mobile Phases
Mobile phase A: 0.05% (v/v) trifluoroacetic acid in 35 mM ammonium chloride in water
Mobile phase B: 0.05% (v/v) trifluoroacetic acid in acetonitrile

Operating Parameters

| Sample | Mobile phase A (%) | Mobile phase B (%) |
|---|---|---|
| The compound of | 0.05% TFA in water | 0.05% TFA in |
| Formula (I) | containing 35 mM | acetonitrile |
| | ammonium chloride | |

Column: ACQUITY UPLC® CSH C18 130 Å, 1.7 μm, 2.1 mm×150 mm
Flow rate: 0.4 mL/min
Detection: 260 nm
Resolution: 4.8 nm
Column temperature: 30° C.
Gradient

| Time (minutes) | Mobile phase A (%) | Mobile phase B (%) |
|---|---|---|
| 0.0 | 99 | 1 |
| 12.4 | 84 | 16 |
| 25.0 | 71 | 29 |
| 30.0 | 50 | 50 |
| 35.0 | 5 | 95 |
| 36.0 | 99.0 | 1.0 |

Injection volume: 14

The samples were prepared at 4 mg/mL in sample diluent, where the sample diluent was 30/20/50 (v/v) sodium acetate buffer at pH 4.5/acetonitrile/ethanol. The samples were then further diluted to 1 mg/mL with sample diluent for injection. Unless otherwise noted, the chemical stability and photodegradation studies used UPLC having the conditions described above for measuring the amount of each compound and impurities.

The results in Table 5 show percent degradation of single agent Formula I free base form. As Table 5 shows, Formula I, free base was chemically and physically stable for approximately nine weeks under the packaging conditions examined (double polyethylene (PE) bags stored at 25° C./60% relative humidity (RH) and 40° C./75% RH). The stability of Formula I is an improvement from Formula I, citrate salt stored at 25° C./60% RH which had a total degradation increase of 1.6% and 2.6% after three and six months respectively. Also shown in Table 5 is there was only fractionally less degradation at 25° C./60% RH than for 40° C./75% RH, while degradation was slightly more at −20° C. In each of the studies performed here, trace amounts of IA and IH were seen but in both cases, their presence did not exceed 0.3%. Further, X-ray powder diffraction pattern showed no change over the nine week period.

TABLE 5
Formula I stability data

| | | | Impurities/Degradation | | | | |
|---|---|---|---|---|---|---|---|
| | | | product (%) | | | Water | Change |
| Storage | Timepoint | Assay | | Formula | RRT 0.34 | content | in XRPD |
| Conditions | (weeks) | (%) | Total | I_{A} | (Formula I_{H}) | (%) | pattern |
| N/A | 0 | 98.8 | 0.4 | 0.29 | 0.15 | 0.04 | N/A |
| −20° C., | 4 | 98.6 | 0.4 | 0.27 | 0.15 | 0.04 | No |
| double PE | | | | | | | |
| bags in | | | | | | | |
| HDPE | | | | | | | |
| bottle | | | | | | | |
| 25° C./60% | 9 | 100.5 | 0.4 | 0.21 | 0.15 | 0.08 | No |
| RH, double | | | | | | | |
| PE bags in | | | | | | | |
| HDPE | | | | | | | |
| bottle | | | | | | | |
| 40° C./75% | 9 | 99.2 | 0.4 | 0.21 | 0.14 | 0.06 | No |
| RH, double | | | | | | | |
| PE bags in | | | | | | | |
| HDPE | | | | | | | |
| bottle | | | | | | | |

XRD = X-ray diffraction
RRT = relative retention time of the individual impurity to the compound of Formula I in the chromatogram

The chemical stability of Formula I in a fixed dose combination formulation tablet of Formulations F4 and F6 were considered. Samples of F4 and F6 were placed in a double polyethylene bag in a high density polyethylene plastic bottle under controlled conditions at either 40° C. at 75% RH and at 25° C. at 60% RH. Stability was measured at 0, 2 month, and 3 month for a solid oral dose form having 10 mg of Formula I, and at 0 and 1 month for a solid oral dose form having 30 mg of Formula I. Formula I and the presence of impurities were measured.

As Table 6 shows, Formula I as contained within F4 and F6 does not experience significant chemical degradation under the conditions examined. The presence of impurities was measured using LC as described above. Also, the presence of impurities (Formulas I_{B}, and I_{D}) remained negligible over the one month duration.

TABLE 6
Formula I of Formulation F4 and Formulation F6 chemical stability and impurities data

| | 30 mg (2.1% w/w) Formula I in F4 | | | 60 mg (4.1% w/w) Formula I in F6 | | |
|---|---|---|---|---|---|---|
| | | 30° C./75% | 40° C./75% | | 30° C./75% | 40° C./75% |
| | | RH | RH | | RH | RH |
| | T = 0 | 1 month | 1 month | T = 0 | 1 month | 1 month |
| | % AN | | | | | |
| 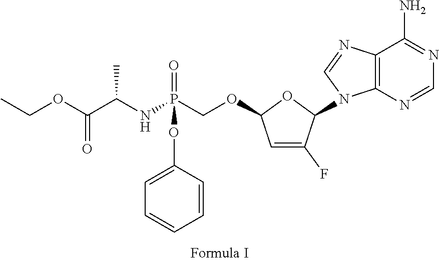 [SMILES: CCOC(=O)[C@H](C)N[P@](=O)(CO[C@@H]1C=C(F)[C@H](n2cnc3c(N)ncnc32)O1)Oc1ccccc1] | 99.7 | 99.7 | 99.5 | 99.6 | 99.6 | 99.5 |

Specified degradation products (% AN)

| | | | | | | |
|---|---|---|---|---|---|---|
| 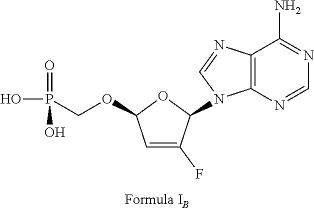 | 0.16 | 0.16 | 0.19 | 0.14 | 0.14 | 0.17 |
| 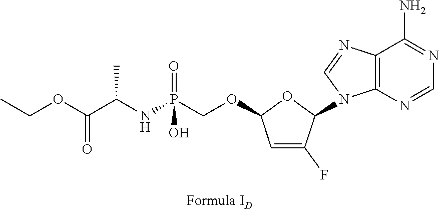 [SMILES: CCOC(=O)[C@H](C)N[P@@](=O)(O)CO[C@@H]1C=C(F)[C@H](n2cnc3c(N)ncnc32)O1] | 0.17 | 0.18 | 0.28 | 0.16 | 0.17 | 0.28 |
| phenol | — | — | — | trace | trace | trace |
| Total Formula I degradation | 0.3 | 0.3 | 0.5 | 0.4 | 0.4 | 0.5 |
| Water content (%) | 1.6 | 1.2 | 1.1 | 1.5 | 1.1 | 1.1 |

% AN = area percentage of the individual peak in the chromatogram relative to the total amount of chromatographic peaks in the chromatogram

The chemical stability of Formula II as contained within F4 and F6 are shown in Table 7. Formula II does not experience significant chemical degradation under the conditions examined.

TABLE 7
Formula II of Formulation F4 and Formulation F6 chemical stability and impurities data

| | 30 mg (2.2% w/w) Formula II in F4 | | | 30 mg (2.2% w/w) Formula IIin F6 | | |
|---|---|---|---|---|---|---|
| | | 30° C./75% | 40° C./75% | | 30° C./75% | 40° C./75% |
| | | RH | RH | | RH | RH |
| | T = 0 | 1 month | 1 month | T = 0 | 1 month | 1 month |
| | % AN | | | | | |
| Formula II | 99.8 | 99.8 | 99.8 | 99.8 | 99.8 | 99.8 |

Specified degradation products (% AN)

| | | | | | | |
|---|---|---|---|---|---|---|
| | 0.2 | 0.2 | 0.2 | 0.2 | 0.2 | 0.2 |
| 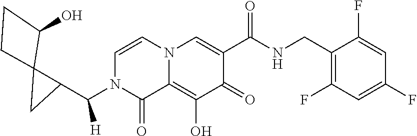 | | | | | | |
| Total Formula II degradation | 0.2 | 0.2 | 0.2 | 0.2 | 0.2 | 0.2 |
| Water content (%) | 1.6 | 1.2 | 1.1 | 1.5 | 1.1 | 1.1 |

% AN = area percentage of the individual peak in the chromatogram relative to the total amount of chromatographic peaks in the chromatogram

The chemical stability of Formula III as contained within F4 and F6 are shown in Table 8. Formula II does not experience significant chemical degradation under the conditions examined.

TABLE 8
Formula III of Formulation F4 and Formulation F6 chemical stability and impurities data

| | 150 mg (10% w/w) Formula | | | 150 mg (10% w/w) Formula | | |
|---|---|---|---|---|---|---|
| | III in F4 | | | III in F6 | | |
| | | 30° C./75% | 40° C./75% | | 30° C./75% | 40° C./75% |
| | | RH | RH | | RH | RH |
| | T = 0 | 1 month | 1 month | T = 0 | 1 month | 1 month |
| | % AN | | | | | |
| Formula III | 99.8 | 99.8 | 99.8 | 99.8 | 99.8 | 99.8 |

Specified degradation products (% AN)

| | | | | | | |
|---|---|---|---|---|---|---|
| 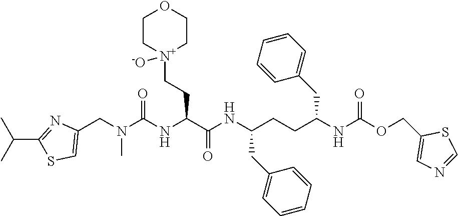 | 0.2 | 0.2 | 0.2 | 0.2 | 0.2 | 0.2 |
| Total Formula III degradation | 0.2 | 0.2 | 0.2 | 0.2 | 0.2 | 0.2 |
| Water content (%) | 1.6 | 1.2 | 1.1 | 1.5 | 1.1 | 1.1 |

% AN = area percentage of the individual peak in the chromatogram relative to the total amount of chromatographic peaks in the chromatogram

The chemical stability of Formula IV as contained within a fixed dose combination formulation tablet (F4 and F6) is shown in Table 9. Formula II does not experience significant chemical degradation under the conditions examined.

TABLE 9
Formula IV of Formulation F4 and Formulation F6 chemical stability and
impurities data

| | % AN | | | | | |
|---|---|---|---|---|---|---|
| | | | | 800 mg (60% w/w) | | |
| | 800 mg (60% w/w) Formula IV | | | Formula IV | | |
| | in F4 | | | in F6 | | |
| | | 30° C./75% | 40° C./75% | | 30° C./75% | 40° C./75% |
| | | RH | RH | | RH | RH |
| | T = 0 | 1 month | 1 month | T = 0 | 1 month | 1 month |
| Formula IV | 99.5 | 99.6 | 99.6 | 99.6 | 99.6 | 99.6 |

Specified degradation products (% AN)

| | | | | | | |
|---|---|---|---|---|---|---|
| RRT 1.11 | 0.3 | 0.3 | 0.3 | 0.3 | 0.3 | 0.3 |
| RRT 1.13 | 0.1 | 0.1 | 0.1 | 0.1 | 0.1 | 0.1 |
| Total Formula IV | 0.2 | 0.2 | 0.2 | 0.2 | 0.2 | 0.2 |
| degradation | | | | | | |
| Water content (%) | 1.6 | 1.2 | 1.1 | 1.5 | 1.1 | 1.1 |

% AN = area percentage of the individual peak in the chromatogram relative to the total amount of chromatographic peaks in the chromatogram
RRT = relative retention time of the individual impurity to the compound of Formula IV in the chromatogram, where the impurity was not characterized
*Structure of the degradation product or impurity has not been proposed or confirmed by further characterization

### Release Profiles for Compounds of Formula I, Formula II, Formula III, and Formula IV.

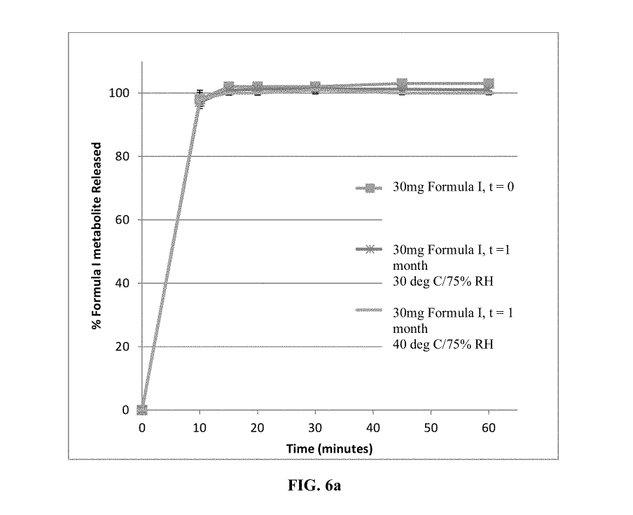

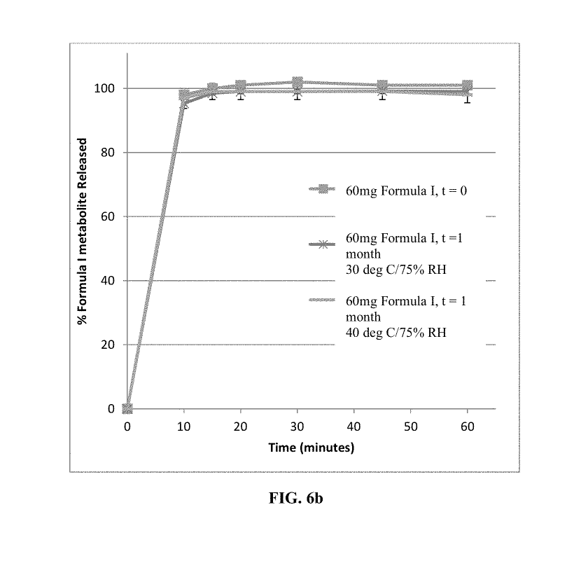

Comparisons of each active ingredient of a fixed dose combination formulation tablet containing compounds of Formula I/II/III/IV were performed by studying percent release of each active ingredient at an initial time and at pre-determined times later. FIGS. 6a and 6b show the release profile of Formula I from Formulation F4 and Formula I from Formulation F6. The Formula I release profile of Formulation F4, data was taken periodically over an hour at an initial time (t=0), and then after the solid oral dosage form was allowed to sit for a two month period at 40° C./75% RH, and finally after the solid oral dosage forms were allowed to sit for three months at 40° C./75% RH and 25° C./60% RH. It should be mentioned here that what was actually measured was the metabolite of Formula I, the compound of Formula I_{B} (FIG. 10). The percent release profiles for Formula I in Formulation F4 (FIG. 6a) formulation did not show significant difference under the different conditions tested. The release profiles for Formula I within Formulation F6 also obtained. At 30° C. and 75% RH and at 40° C. and 75% RH, the release profile for Formulas I of Formulation F6 were near identical for the entire length of time data was taken.

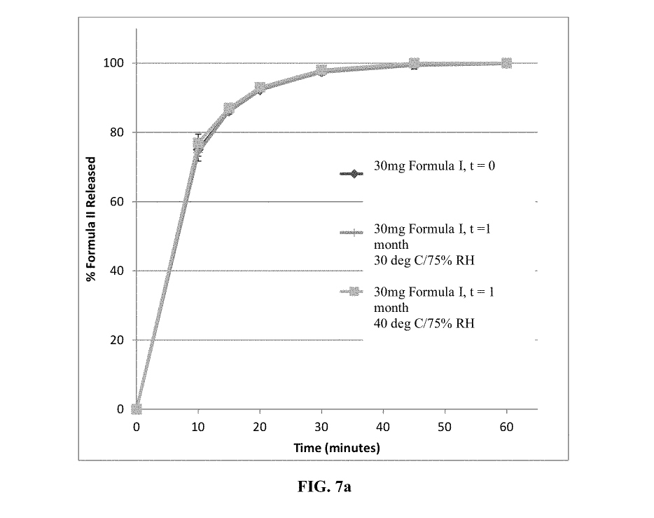

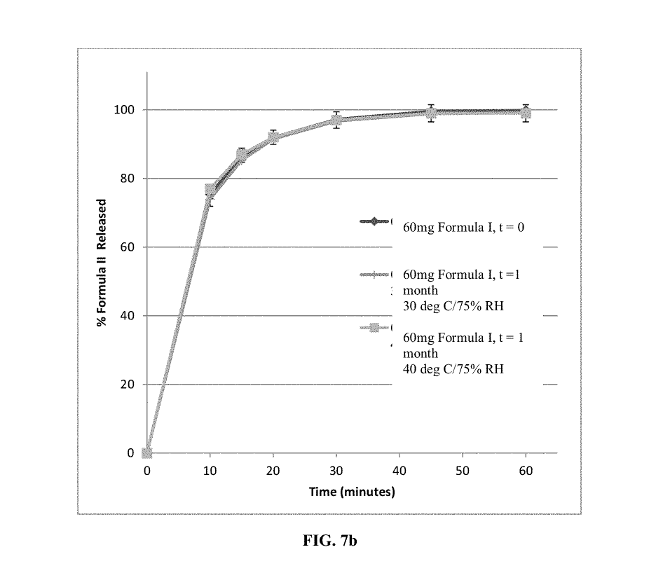

FIGS. 7a and 7b show the release profile for Formula II for the fixed dose combination formulation tablet of Formulations F4 and F6, respectively. The Formula II release profile of Formulation F4, data was taken periodically over an hour period at an initial time (t=0), and then after the solid oral dosage form was allowed to sit for a two month period at 40° C./75% RH, and finally after the solid oral dosage forms were allowed to sit for three months at 40° C./75% RH and 25° C./60% RH. As shown in FIG. 7a, the four percent release curves (t =0; t at 2 months, 40° C./75% RH; time at 3 months, 40° C./75% RH and 25° C./60% RH) are comparable in their percent release profile between time zero and sixty minutes (FIG. 7a). Formula II percent release profiles for Formulation F6 at time at zero and time after one month at 40° C./75% RH, were also comparable over the course of the sixty minute period when data was taken (FIG. 7b).

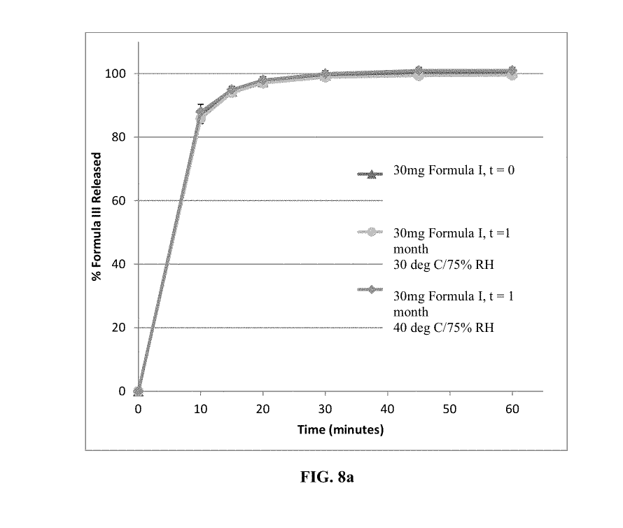

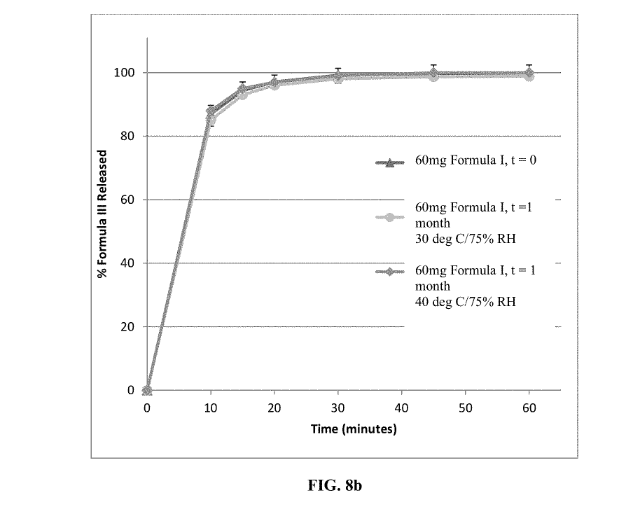

FIGS. 8a and 8b show the release profile for Formula III for the fixed dose combination formulation tablet of Formulations F4 and F6. The release profile of Formula III of F4, data was taken periodically over an hour period at an initial time (t=0), and then after the solid oral dosage form was allowed to sit for a two month period at 40° C./75% RH, and finally after the solid oral dosage forms were allowed to sit for three months at 40° C./75% RH and 25° C./60% RH. As FIG. 8a shows, percent release data taken for all four profiles were comparable. The percent release profile for Formula III within Formulation F6 was taken at zero time and then again after one month at 30° C. and 75% RH and also at 40° C. and 75% RH (FIG. 8b). The percent release profile for the t at zero to t at about 60 minutes were comparable for the different conditions tested.

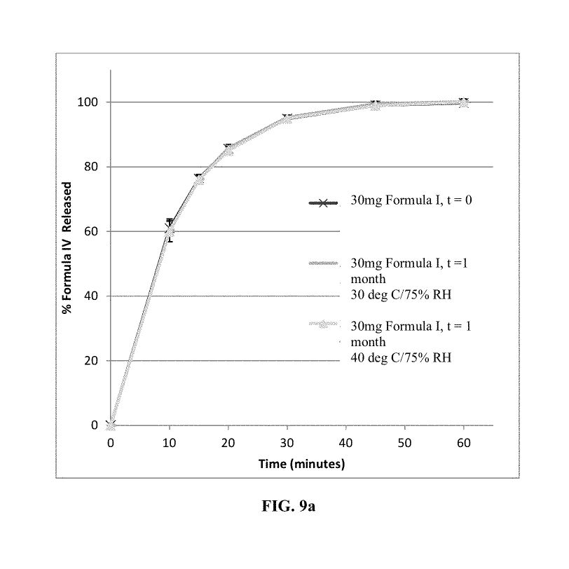

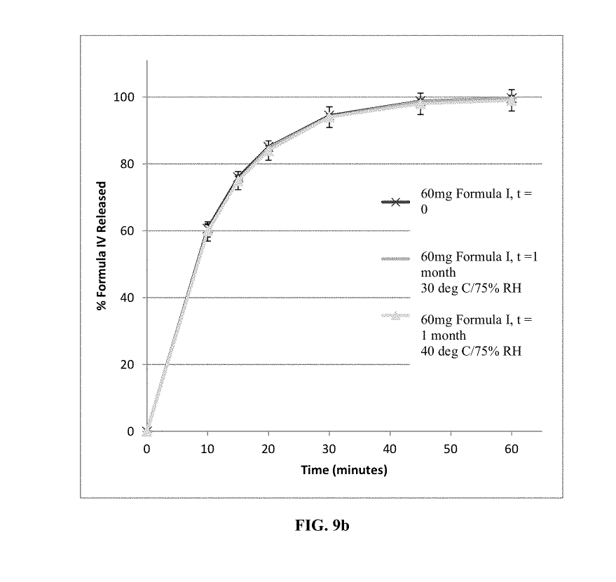

FIGS. 9a and 9b show the release profile of Formula IV within the fixed dose combination formulation tablet of Formulations F4 and F6. Similar to the previous percent release studies, data was taken over a sixty minute period for t at zero, time after two months at 40° C./75% RH, and again after three months at 40° C./75% RH and 25° C./60% RH for Formula IV within Formulation F4. As FIG. 9a shows, the percent release for Formula IV at the different time periods and conditions were comparable. Similarly, the release profile of Formula IV within Formulation F6 at 30° C. and 75% RH and 40° C. and 75% RH was comparable over the time course studied (FIG. 9b).

As the data shows, each active ingredient within both the fixed dose combination formulation tablets (Formulations F4 and F6) released similarly (both in amount and characteristics) from zero time up to three months and up to one month for Formulations F4 and F6, respectively.

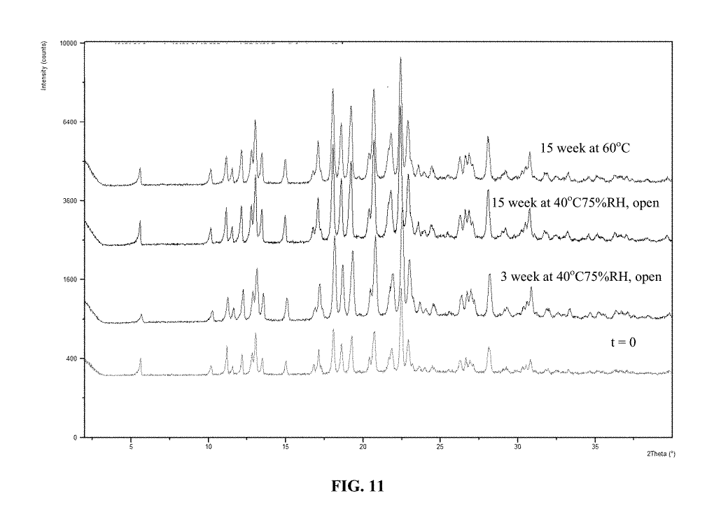

The stability of the compound of Formula I, free base, was also evaluated by X-ray powder diffraction spectra (XRPD) taken at an initial time (t=0), after 3 weeks at 40 C/75% RH in an open container, and again after 15 weeks, at 60 C in a closed container and 40 C/75% RH in an open container. The diffractogram of XRPD is typically represented by a diagram plotting the intensity of the peaks versus the location of the peaks, i.e., diffraction angle 20 (two-theta) in degrees. The characteristic peaks of a given XRPD can be selected according to the peak locations and their relative intensity to conveniently distinguish this crystalline structure from others. XRPD patterns were collected on a PANanalytical XPERT-PRO diffractometer at ambient conditions under the following experimental settings: 45 KV; 40 mA, Kα1=1.5406 A; scan range 2 to 40°; step size 0.0084 or 0.0167°; measurement time: 5 minutes. XRPD patterns were collected at ambient temperatures unless otherwise noted. Comparison of the spectra taken under these varied conditions is shown in FIG. 11. The series of spectra show no change for the samples at the different temperature, relative humidity and whether the container was closed or open; even the fine features of the initial spectrum are not lost after 15 weeks. As the spectra show, Formula I is physically stable after 15 weeks at both 60 C in a closed or in an open container.

### Therapeutic Methods

The solid oral dosage forms (in particular tablets) disclosed herein are used for treatment or prevention of HIV infection (e.g. HIV-1 infection). In certain embodiments, the solid oral dosage forms (in particular tablets) disclosed herein are used for treatment of HIV infection (e.g. HIV-1 infection). In certain embodiments, the solid oral dosage forms (in particular tablets) disclosed herein are used for pre-exposure prophylaxis (PrEP) to reduce the risk of sexually acquired HIV-1. In certain embodiments, the solid oral dosage forms (in particular tablets) disclosed herein are used to treat a treatment-experienced subject. In some embodiments, the treatment experienced subject has a resistance mutation (e.g., one or more thymidine analogue mutations (TAM) and/or other nucleoside RT inhibitor (NRTI) resistance mutation such as M184V, K65R, L74V).

Accordingly, methods for treating a subject infected with HIV are provided, comprising administering a solid oral dosage form disclosed herein (in particular a tablet) to the subject. Similarly, a solid oral dosage form (in particular a tablet) is provided for use in such treatment methods. Also provided is the use of solid oral dosage form disclosed herein in the manufacture of an oral dosage form disclosed herein (in particular a tablet) for treatment of HIV infection. For example, also provided is the use of (a) the compound of Formula I, or a pharmaceutically acceptable salt thereof, (b) the compound of Formula II, or a pharmaceutically acceptable salt thereof, (c) cobicistat, or a pharmaceutically acceptable salt thereof, and (d) darunavir, or a pharmaceutically acceptable salt or solvate thereof, in the manufacture of an oral dosage form disclosed herein (in particular a tablet) for treatment of HIV infection. Similarly, in some embodiments, the invention provides the use of (a) compound of Formula I, or a pharmaceutically acceptable salt thereof, (b) the compound of Formula II, or a pharmaceutically acceptable salt thereof, (c) cobicistat, or a pharmaceutically acceptable salt thereof, and (d) darunavir, or a pharmaceutically acceptable salt or solvate thereof, in the manufacture of an oral dosage form disclosed herein (in particular a tablet) for treatment of HIV infection in treatment-experienced patients.

In certain embodiments, the solid oral dosage forms (in particular tablets) disclosed herein are used for pre-exposure prophylaxis (PrEP) to reduce the risk of sexually acquired HIV-1. Accordingly, methods for preventing infection in a subject at risk of infection with HIV are provided, comprising administering a solid oral dosage form disclosed herein (in particular a tablet) to the subject. Similarly, a solid oral dosage form disclosed herein (in particular a tablet) is provided for use in such treatment methods. The invention also provides the use of the solid oral dosage forms disclosed herein, in the manufacture of an oral dosage form disclosed herein (in particular a tablet) for prevention of HIV infection in a subject at risk for infection (e.g., the use of (a) the compound of Formula I, or a pharmaceutically acceptable salt thereof, (b) the compound of Formula II, or a pharmaceutically acceptable salt thereof, (c) cobicistat, or a pharmaceutically acceptable salt thereof, and (d) darunavir, or a pharmaceutically acceptable salt or solvate thereof, in the manufacture of an oral dosage form disclosed herein (in particular a tablet) for prevention of HIV infection in a subject at risk for infection). In some embodiments, the invention provides the use of the solid oral dosage forms disclosed herein, in the manufacture of an oral dosage form disclosed herein (in particular a tablet) for prevention of HIV infection (e.g., provides the use of (a) the compound of Formula I, or a pharmaceutically acceptable salt thereof, (b) the compound of Formula II, or a pharmaceutically acceptable salt thereof, (c) cobicistat, or a pharmaceutically acceptable salt thereof, and (d) darunavir, or a pharmaceutically acceptable salt or solvate thereof, in the manufacture of an oral dosage form disclosed herein (in particular a tablet) for prevention of HIV infection).

The methods involve administering an oral dosage form disclosed herein (in particular a tablet) to the subject, typically a human, and will generally involve repeated administrations, typically once daily. The treatment may be prophylactic or therapeutic treatment.

### General

The term “comprise” and variations thereof, such as “comprises” and “comprising”, are to be construed in an open, inclusive sense, that is as “including, but not limited to”.

The term “between” with reference to two values includes those two values e.g. the range “between” 10 mg and 20 mg encompasses e.g. 10, 11, 12, 13, 14, 15, 16, 17, 18, 19 and 20 mg.

The term “about” used in connection with a quantity is inclusive of the stated value and has the meaning dictated by the context (e.g., includes the degree of error associated with measurement of the particular quantity). For example, in certain nonlimiting example the term “about” in relation to a numerical value x refers to x±10%, x±5%, or x±1%.

“% w/w” means the weight of a component as a percentage of the total weight of e.g. a layer or dosage form in which the component is present. For example, a composition comprising “5% w/w X” refers to a composition in which the weight of component X is 5% of the total weight of the composition.

Reference throughout this specification to “one embodiment” or “an embodiment” means that a particular feature, structure or characteristic described in connection with the embodiment is included in at least one embodiment provided herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “pharmaceutically acceptable” with respect to a substance refers to that substance which is generally regarded as safe and suitable for use without undue toxicity, irritation, allergic response, and the like, commensurate with a reasonable benefit/risk ratio. “Pharmaceutically acceptable” with regard to excipients includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” refers to a salt of a compound that is pharmaceutically acceptable and that possesses (or can be converted to a form that possesses) the desired pharmacological activity of the parent compound. Such salts include acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, lactic acid, maleic acid, malonic acid, mandelic acid, methanesulfonic acid, 2-napththalenesulfonic acid, oleic acid, palmitic acid, propionic acid, stearic acid, succinic acid, tartaric acid, p-toluenesulfonic acid, trimethylacetic acid, and the like, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quaternized ammonium salts. Representative non-limiting lists of pharmaceutically acceptable salts can be found in S. M. Berge et al., J. Pharma Sci., 66 (1), 1-19 (1977), and Remington: The Science and Practice of Pharmacy, R. Hendrickson, ed., 21 st edition, Lippincott, Williams & Wilkins, Philadelphia, Pa., (2005), at p. 732, Table 38-5, both of which are hereby incorporated by reference herein.

As used herein, the term “salts” includes co-crystals. The term “co-crystal” refers to a crystalline compound comprising two or more molecular components, e.g. wherein proton transfer between the molecular components is partial or incomplete.

The term “solvate” means a molecular complex comprising a compound and one or more pharmaceutically acceptable solvent molecules. Examples of solvent molecules include water and C₁₋₆ alcohols, e.g. ethanol. When the solvate is water, the term “hydrate” may be used.

“Treating” and “treatment” of a disease include the following:
(1) preventing or reducing the risk of developing the disease, i.e. causing the clinical symptoms of the disease not to develop in a subject that may be exposed to or predisposed to the disease but does not yet experience or display symptoms of the disease,
(2) inhibiting the disease, i.e. arresting or reducing the development of the disease or its clinical symptoms, and
(3) relieving the disease, i.e. causing regression of the disease or its clinical symptoms.

The term “effective amount” refers to an amount that may be effective to elicit the desired biological or medical response, including the amount of a compound that, when administered to a subject for treating a disease, is sufficient to effect such treatment for the disease. The effective amount will vary depending on the compound, the disease and its severity and the age, weight, etc. of the subject to be treated. The effective amount can include a range of amounts.

All publications, patents and patent applications are incorporated by reference in their entirety, as though individually incorporated by reference. The invention has been described with reference to various specific and preferred embodiments and techniques. However, it should be understood that many variations and modifications may be made while remaining within the spirit and scope of the invention.

### EXAMPLES

The following examples are provided for purposes of illustration, not limitation.

### Example 1

### Tablet Formulation F1

A formulation (tablet F1 ) was prepared as described in the table and description below:

| | Tablet Formulation F1 | |
|---|---|---|
| Component | Mass (mg/tablet) | % w/w/tablet |

Intragranular

| | | |
|---|---|---|
| Compound of Formula I, free base | 10.00 | 0.71 |
| Compound of Formula II, sodium salt | 52.49 | 3.75 |
| Compound of Formula III (on SiO₂) | 288.55 | 20.61 |
| Compound of Formula IV, free base | 800.00 | 57.14 |
| Microcrystalline cellulose, Avicel | 143.96 | 10.28 |
| PH102 | | |
| Crospovidone, Polyplasdone XL | 84.00 | 6.00 |
| Magnesium Stearate | 10.50 | 0.75 |

Extragranular

| | | |
|---|---|---|
| Magnesium Stearate | 10.50 | 0.75 |
| Total Weight | 1400 | |

The tablets of Formulation F1 were film coated with 42 mg Opadry II Brown 85F165072 (3.0% w/w). The total weight of the film coated tablets was 1442 mg.

The compounds of Formula I, II, III (each, Gilead Sciences, Inc.), and IV (Zhejiang Jiangbei Pharmaceutical Co., Ltd.) were blended with intragranular excipients (microcrystalline cellulose, crospovidone, and magnesium stearate), roller compacted, milled, and final blended with the extragranular portion of the magnesium stearate to yield a final powder blend for compression. The final powder blend was compressed into tablet cores, which were film-coated as described above to a target weight gain of 3%.

### Example 2

### Tablet Formulation F2

A formulation (tablet F2) was prepared as described in the table and description below:

| | Tablet Formulation F2 | |
|---|---|---|
| Component | Mass (mg/tablet) | % w/w/tablet |

Intragranular

| | | |
|---|---|---|
| Compound of Formula I, free base | 10.00 | 0.68 |
| Compound of Formula II, sodium salt | 52.49 | 3.62 |
| Compound of Formula III (on SiO₂) | 288.55 | 19.90 |
| Compound of Formula IV, ethanolate | 867.3 | 59.81 |
| solvate | | |
| Microcrystalline cellulose, Avicel | 123.0 | 8.49 |
| PH102 | | |
| Crospovidone, Polyplasdone XL | 87.00 | 6.00 |
| Magnesium Stearate | 10.9 | 0.75 |

Extragranular

| | | |
|---|---|---|
| Magnesium Stearate | 10.9 | 0.75 |
| Total Weight | 1450 | |

The tablets of Formulation F2 were film coated with 43.5 mg Opadry II Brown 85F165010 (3.0% w/w). The total weight of the film coated tablets was 1494 mg.

The compounds of Formula I, II, III (each, Gilead Sciences, Inc.), and IV (Zhejiang Jiangbei Pharmaceutical Co., Ltd.) were blended with intragranular excipients (microcrystalline cellulose, crospovidone, and magnesium stearate), roller compacted, milled, and final blended with the extragranular portion of the magnesium stearate to yield a final powder blend for compression. The final powder blend was compressed into tablet cores, which were film-coated as described above to a target weight gain of 3%.

### Example 3

### Tablet Formulation F4

### Tablet Formulation F4

A formulation (tablet F4) was prepared as described in the following table and description below:

| | Tablet Formulation F4 | |
|---|---|---|
| Component | Mass (mg/tablet) | % w/w/tablet |

Intragranular

| | | |
|---|---|---|
| Compound of Formula I, free base | 30.0 | 2.0 |
| Compound of Formula II | 31.5 | 2.1 |
| Compound of Formula III | 288.6 | 19.3 |
| adsorbed onto SiO₂ | | |
| Compound of Formula IV, ethanolate | 867.3 | 58.0 |
| Microcrystalline cellulose, Avicel | 130.9 | 8.8 |
| PH102 | | |
| Crospovidone, Polyplasdone XL | 87.0 | 5.8 |
| Magnesium Stearate | 7.3 | 0.50 |

Extragranular

| | | |
|---|---|---|
| Magnesium Stearate | 7.3 | 0.50 |

Film coating

| | | |
|---|---|---|
| Opadry II Brown 85F165072 | 43.5 | 3.0 |
| Total Weight of film-coated tablet | 1494 | 100 |

The tablets of Formulation F4 were film coated with 42 mg Opadry II Brown 85F165072 (3.0% w/w). The total weight of the film coated tablets was 1494 mg.

The compounds of Formula I, II, III (each, Gilead Sciences, Inc.), and IV (Janssen Pharmaceuticals, Inc.) were blended with intragranular excipients (microcrystalline cellulose, crospovidone, and magnesium stearate), roller compacted, milled, and final blended with the extragranular portion of the magnesium stearate to yield a final powder blend for compression. The final powder blend was compressed into tablet cores, which were film-coated as described above to a target weight gain of 3%.

### Example 4

### Tablet Formulation F6

A formulation (tablet F4) was prepared as described in the table and description below:

| | Tablet Formulation F6 | |
|---|---|---|
| | Mass | % w/w/tablet |
| Component | (mg/tablet) | (uncoated) |

Intragranular

| | | |
|---|---|---|
| Compound of Formula I, free base | 60.0 | 4.0 |
| Compound of Formula II | 31.5 | 2.17 |
| Compound of Formula III | 288.6 | 19.3 |
| adsorbed on SiO₂ | | |
| Compound of Formula IV, ethanolate | 867.3 | 58.0 |
| Microcrystalline cellulose, Avicel | 100.9 | 6.86 |
| PH102 | | |
| Crospovidone, Polyplasdone XL | 87.0 | 5.8 |
| Magnesium Stearate | 7.3 | 0.50 |

Extragranular

| | | |
|---|---|---|
| Magnesium Stearate | 7.3 | 0.50 |

Film coating

| | | |
|---|---|---|
| Opadry II Brown 85F165072 | 43.5 | 3.0 |
| Total Weight of film-coated tablet | 1494 | 100 |

The tablets of Formulation F6 were film coated with 43.5 mg Opadry II Brown 85F165010 (3.0% w/w). The total weight of the film coated tablets was 1494 mg. The total weight of the uncoated tablets was 1450 mg.

The compounds of Formula I, II, III (each, Gilead Sciences, Inc.), and IV (Janssen Pharmaceuticals, Inc.) were blended with intragranular excipients (microcrystalline cellulose, crospovidone, and magnesium stearate), roller compacted, milled, and final blended with the extragranular portion of the magnesium stearate to yield a final powder blend for compression. The final powder blend was compressed into tablet cores, which were film-coated as described above to a target weight gain of 3%.

### Example 5

### Tablet Formulation F7 and F8

Formulations (tablet F7 and F8) was prepared as described in the table and description below:

The tablets of Formulations F7 and F8 were film coated with 4 mg Opadry II White 85F18422. The total weight of the film coated tablets were 104 mg for the F7 formulation and 312 mg for the F8 Formulation.

### Example 6

### Tablet Formulation F9 and F10

Formulations (tablet F9 and F10) was prepared as described in the table and description below:

The tablets of Formulations F9 and F10 were film coated with 8 mg and 4.8 mg of Opadry II Yellow 85F92259, respectively. The total weight of the film coated tablets were 208 mg and 124.8 mg respectively.

### Example 7

Formulation (F11) Used in PK Studies

### Example 8

F12, Single Agent Formula I, Single Agent Formula II, and Co-Formulated Formula III and Formula IV Used In Comparative Studies with Formula I/Formula II/Formula III/Formula IV Fixed close Combination Formulation Tablet

| Ingredient for F10 | mg |
|---|---|
| Formula I | 15 mg |
| Formula II | 7.5 mg |
| co-formulated Formula III and Formula IV | 37 & 200 |

### Example 9

### Manufacturing Process

The manufacturing/packaging procedure for the tablets described herein is divided into the following unit processes:
1. mixing of the compounds of Formula I, II, III, and IV drug substances with intergranular excipients, roller compaction or slugging, milling, and blending with extragranular excipients to yield the final powder blend;
2. tablet compression to yield tablet cores;
3. tablet film-coating to yield film-coated tablets; and
4. packaging.

The manufacturing process steps to produce the final drug product are depicted in FIG. 1 and detailed below.

### Final Powder Blend (Dispensing, Blending, Dry Granulation, Milling, Final Blending)

1. Weigh the compounds of Formula I, II, III, and IV and the excipients (microcrystalline cellulose, crospovidone). Correct the weight of the compounds of Formula I, II, III, and IV based on the drug content factor (DCF), with a concomitant reduction in the weight of microcrystalline cellulose.
2. Blend in intergranular portion of magnesium stearate to the tumble blender and blend.
3. Dry granulate the resulting blend using a roller compactor or slug the resulting blend and mill.
4. Add extragranular magnesium stearate and blend.

### Tableting

5. Compress the final powder blend with an appropriate main compression force to achieve a target hardness of at least 23 kP).

### Film-Coating

6. Prepare a suspension of Opadry® II Brown 85F165072 or 85F165010. Film-coat the tablet cores to achieve the target tablet weight gain of 3% (range 2-4%). Dry film-coated tablets prior to cooling and discharge.

All publications, patents and patent applications are incorporated by reference in their entirety, as though individually incorporated by reference. The invention has been described with reference to various specific and preferred embodiments and techniques. However, it should be understood that many variations and modifications may be made while remaining within the spirit and scope of the invention.

### Example 10

### Pharmacokinetic Analysis Following Oral Administration of Subjects

Pharmacokinetic analysis was performed on various test compounds following intravenous or oral administration to beagle dogs.

For pharmacokinetic analysis of orally administered compounds, the test compounds were formulated as an aqueous suspension in 0.1% Tween 20, 0.5% HPMC LV100 in deionized water at 1 mg/kg.

Each dosing group consisted of 3-6 male, non-naïve purebred beagle dogs. At dosing, the animals weighed between 7 to 13 kg. The animals were fasted overnight prior to dose administration and up to 4 hr after dosing. Each animal received a single 6 μg/kg intramuscular injection of pentagastrin approximately 30 minutes prior to test article administration. For studies of oral administration, the test article was administered as a fixed dose of Formula I, Formula II, Formula III and Formula IV, respectively, followed by ˜10 mL of water to facilitate swallowing.

For pharmacokinetic analysis of intravenously administered compounds, serial venous blood samples (approximately 1 mL each) were taken from each animal at 0, 0.25, 0.50, 1, 2, 4, 6, 8, 12, and 24 hours after dosing. The blood samples were collected into Vacutainer™ tubes containing EDTA-K2 as the anti-coagulant and were immediately placed on wet ice pending centrifugation for plasma.

### Example 11

Drug Combination Antiviral Assay for the Compound of Formula I In Combination with Bictegravir

Compounds were tested pair-wise in high-throughput 384-well assay format using an in vitro cytopathic effect (CPE) assay that quantifies protection from HIV-1 induced cell death. Individual test compounds were serially diluted (1:1.7) in DMSO generating 7 or 9-points. Serially diluted compounds within a pair are arranged orthogonally with respect to each other in separate plates. Two compounds in a pair are combined in the assay plate (black, Greiner) by transferring 200 nl of each compound using the acoustic liquid disperser Echo (Labcyte). Positive controls, such as elvitegravir (EVG) and AZT, as well as a negative control (DMSO) were included in every assay plate to define 100% and 0% protection, respectively. Final DMSO concentration in the assay is 0.5%. MT-2 cells were bulk infected with HIV-1 IIIb (250× diluted) at 100 μl virus per 2×10⁶ cells and incubated for 3 hours at 37° C. MT-2 cells are subsequently added to the plate at 3,000 cells per 804 media (1640 RPMI supplemented with 10% fetal bovine serum, 100 Units/mL Penicillin, 100 μg/mL Streptomycin) using a Biotek uFlow Workstation. Assay plates are incubated for 5-days at 37° C. in a humidified incubator. To measure the cytopathic effect of HIV, 40 μL Cell Titer Glo was added to each well and the resulting luminescence signal is read with the Envision plate reader (Perkin Elmer). Data were normalized to positive and negative control in each plate and expressed as % CPE Protection.

### Data Analysis

The combination effect of each tested pair of inhibitors was determined by the analysis of % CPE Protection from the anti-HIV-1 cytopathic assay using MacSynergyTM II program (University of Michigan, Ann Arbor, Mich.). Prichard, M. N., K. R. Aseltine, and C. Shipman, Jr, MacSynergy™ II, Version 1.0, 1993, University of Michigan, Ann Arbor, Mich. This program is based on an algorithm previously described by Prichard and Shipman (see M. N. Prichard and C. Shipman, Jr., A three-dimensional model to analyze drug-drug interactions, Antiviral Res, 1990 14 (4-5): p. 181-205; M. N. Prichard, L. E. Prichard, and C. Shipman, Jr., Strategic design and three-dimensional analysis of antiviral drug combinations, Antimicrob. Agents Chemother., 1993 37 (3): p. 540-5), and allows for the assessment of the degree of synergy, additivity, or antagonism for each of the tested pair-wise combinations of antiretrovirals. The program defines the combination effect according to a specific numeric value of combination volume (μM¹%) calculated from the antiviral inhibition data (Table A). To determine the nature and the degree of the analyzed interactions, combination volume values were calculated at their 95% confidence level.

TABLE A
Definition of Drug Combination Effects

| Combination volume [μM² %] | Combination effect |
|---|---|
| ≥100 | Highly synergistic |
| ≥50 to <100 | Slightly synergistic |
| ≥−50 to <50 | Additive |
| ≥−100 to <−50 | Slightly antagonistic |
| <−100 | Highly antagonistic |

### Results

The antiviral activity of the compound of formula I in combination with bictegravir was evaluated in MT-2 cells infected with HIV-1 IIIb. The compound of formula I acted synergistically with bictegravir (Table 2). When combined with itself, the compound of formula I showed additive anti-HIV activity. No antagonism was observed between the compound of formula I and bictegravir. The synergy and antagonism controls used were EVG/TAF and RBV/d4 T, respectively.

TABLE 1
In vitro Anti-HIV-1 Activity of the Compound of Formula I in
Combination with Bictegravir

| | Synergy/Antagonism^{a} | | Combination |
|---|---|---|---|
| Drug combination^{b} | Type | Mean | effect |
| A compound of | Synergy | 5 ± 6 | Additive |
| formula I + A | Antagonism | −12 ± 9 | |
| compound of | | | |
| formula I | | | |
| A compound of | Synergy | 124 ± 18 | Highly Synergistic |
| formula I + | Antagonism | −4 ± 6 | |
| Bictegravir | | | |
| EVG + TAF | Synergy | 164 ± 24 | Highly Synergistic |
| | Antagonism | −3 ± 3 | |
| RBV + d4T | Synergy | 0 ± 0 | Highly Antagonistic |
| | Antagonism | −398 ± 23 | |

^{a}The synergy/antagonism volumes represent the mean of at least 3 independent experiments done in triplicates.
^{b}RBV = ribavirin, d4T = stavudine, TAF = tenofovir alafenamide, EVG = elvitegravir.

In summary, co-dosing the compound of Formula I with bictegravir results in strong synergistic antiviral activity when tested in vitro using HIV-1 IIIb and MT2 cells.

All publications, patents and patent applications are incorporated by reference in their entirety, as though individually incorporated by reference. The invention has been described with reference to various specific and preferred embodiments and techniques. However, it should be understood that many variations and modifications may be made while remaining within the spirit and scope of the invention.

1. A solid oral dosage form comprising:
about 0.5% to about 2.5% w/w of a compound of Formula I: 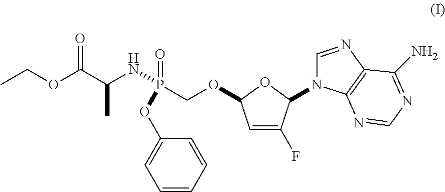
or a pharmaceutically acceptable salt thereof;
about 2% to about 6% w/w of a compound of Formula II: 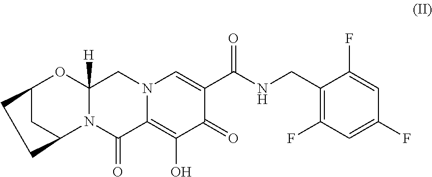 [SMILES: O=C(NCc1c(F)cc(F)cc1F)c1cn2c(c(O)c1=O)C(=O)N1[C@H]3CC[C@H](C3)O[C@@H]1C2]
or a pharmaceutically acceptable salt thereof;
about 10% to about 30% w/w a compound of Formula III: 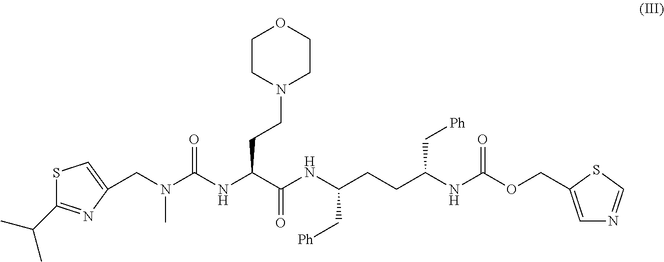 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]
or a pharmaceutically acceptable salt thereof; and
about 40% to about 75% w/w of a compound of Formula IV: 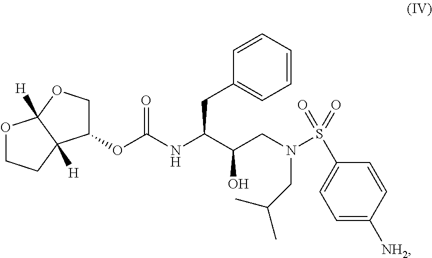 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]
or a pharmaceutically acceptable salt or solvate thereof.

2. A solid oral dosage form comprising:
(a) about 1.0% to about 3.1% w/w of the compound of Formula I, or a pharmaceutically acceptable salt thereof;
(b) about 1.0% to about 4.0% w/w of the compound of Formula II, or a pharmaceutically acceptable salt thereof;
(c) about 4% to about 18% w/w the compound of Formula III, or a pharmaceutically acceptable salt thereof;
(d) about 50% to about 70% w/w of the compound of Formula IV, or a pharmaceutically acceptable salt or solvate thereof.

3. The solid oral dosage form of claim 1, comprising:
(a) about 1.5% to about 2.5% w/w of the compound of Formula I, or a pharmaceutically acceptable salt thereof;
(b) about 2.0% to about 3.0% w/w of the compound of Formula II, or a pharmaceutically acceptable salt thereof;
(c) about 7% to about 12% w/w the compound of Formula III, or a pharmaceutically acceptable salt thereof;
(d) about 50% to about 65% w/w of the compound of Formula IV, or a pharmaceutically acceptable salt or solvate thereof.

4. A solid oral dosage form comprising:
(a) about 1.0% to about 3.1% w/w of the compound of Formula I, or a pharmaceutically acceptable salt thereof;
(b) about 1.0% to about 4.0% w/w of the compound of Formula II, or a pharmaceutically acceptable salt thereof;
(c) about 15% to about 25% w/w of the compound of Formula III, or a pharmaceutically acceptable salt thereof adsorbed onto a solid carrier;
(d) about 50% to about 70% w/w of the compound of Formula IV, or a pharmaceutically acceptable salt or solvate thereof.

5. The solid oral dosage form of claim 4, wherein the solid carrier comprises silica particles.

6. The solid oral dosage form of claim 1, wherein the compound of Formula III is adsorbed onto silica particles.

7. (canceled)

8. The solid oral dosage form of claim 1, comprising:
(a) about 20 mg to about 40 mg of the compound of Formula I, or a pharmaceutically acceptable salt thereof;
(b) about 20 mg to about 45 mg of the compound of Formula II, or a pharmaceutically acceptable salt thereof;
(c) about 125 mg to about 250 mg of the compound of Formula III, or a pharmaceutically acceptable salt thereof;
(d) about 550 mg to about 1100 mg of the compound of Formula IV, or a pharmaceutically acceptable salt or solvate thereof;
(e) about 90 mg to about 165 mg of microcrystalline cellulose;
(f) about 60 mg to about 110 mg of crospovidone; and
(g) about 5 mg to about 20 mg of magnesium stearate.

9. The solid oral dosage form of claim 1, comprising:
(a) about 28 mg to about 32 mg of the compound of Formula I, or a pharmaceutically acceptable salt thereof;
(b) about 25 mg to about 37 mg of the compound of Formula II, or a pharmaceutically acceptable salt thereof;
(c) about 135 mg to about 180 mg of the compound of Formula III, or a pharmaceutically acceptable salt thereof, and silicon dioxide particles;
(d) about 700 mg to about 1000 mg of the compound of Formula IV, or a pharmaceutically acceptable salt or solvate thereof;
(e) about 110 mg to about 150 mg of microcrystalline cellulose;
(f) about 60 mg to about 110 mg of crospovidone; and
(g) about 5 mg to about 20 mg of magnesium stearate.

10. The solid oral dosage form of claim 1, comprising:
(a) about 28 mg to about 32 mg of the compound of Formula I, or a pharmaceutically acceptable salt thereof;
(b) about 25 mg to about 37 mg of the compound of Formula II, or a pharmaceutically acceptable salt thereof;
(c) about 230 mg to about 350 mg of the compound of Formula III, or a pharmaceutically acceptable salt thereof adsorbed onto a solid carrier,
(d) about 700 mg to about 1000 mg of the compound of Formula IV, or a pharmaceutically acceptable salt or solvate thereof;
(e) about 120 mg to about 150 mg of microcrystalline cellulose;
(f) about 60 mg to about 110 mg of crospovidone; and
(g) about 5 mg to about 20 mg of magnesium stearate.

11. The solid oral dosage form of claim 1, wherein the dosage form is a tablet.

12. The solid oral dosage form of claim 1, further comprising a film coating.

13. The solid oral dosage form of claim 1, wherein the dosage form has a total weight of about 1.5 g.

14. The solid oral dosage form of claim 1, wherein the dosage form has a total weight of about 1400 mg to about 1600 mg.

15. (canceled)

16. The solid oral dosage form of claim 1, wherein the dosage form includes less than about 600 mg of excipients.

17. The solid oral dosage form of claim 13, wherein the dosage form includes less than about 300 mg of excipients.

18. (canceled)

19. A method of therapeutic treatment of an HIV infection comprising administering to a subject a solid oral dosage form according to claim 1.

20. The method of claim 19, wherein the subject is a treatment-experienced subject.

21. The method of claim 20, wherein the treatment-experienced subject has a resistance mutation selected from a thymidine analogue mutation (TAM), M184V, K65R, and L74V.

22.-56. (canceled)

57. The solid oral dosage form of claim 16, wherein the dosage form includes less than about 300 mg of excipients.
